(19) United States
(12) Patent Application Publication
(10) Pub. No.: US 2024/0002852 A1
(43) Pub. Date: Jan. 4, 2024

(54) COMPOUNDS FOR MODULATING CHMP7

(71) Applicant: Ionis Pharmaceuticals, Inc., Carlsbad, CA (US)

(72) Inventor: Huynh-Hoa Bui, San Diego, CA (US)

(73) Assignee: Ionis Pharmaceuticals, Inc., Carlsbad, CA (US)

(21) Appl. No.: 18/247,311

(22) PCT Filed: Oct. 1, 2021
(86) PCT No.: PCT/US2021/053107

§ 371 Date: Mar. 30, 2023

Related U.S. Application Data
(60) Provisional application No. 63/139,715, filed on Jan. 20, 2021.
(60) Provisional application No. 63/119,448, filed on Nov. 30, 2020.
(60) Provisional application No. 63/086,576, filed on Oct. 1, 2020.

(51) Int. Cl. C12N 15/113

(52) U.S. Cl. CPC C12N 15/113; C12N 2310/11; C12N 2310/341; C12N 2310/315; C12N 2310/3341; C12N 2310/321

(57) ABSTRACT

Provided are compounds and pharmaceutical compositions for reducing the amount or activity of Charged Multivesicular Body Protein 7 (CHMP7) RNA in a cell or subject, and in certain instances reducing the amount of CHMP7 protein in a cell or subject. Such compounds and pharmaceutical compositions are useful to ameliorate diseases or conditions associated with aberrant activation of Endosomal Sorting Complexes Required for Transport-III proteins.

### SEQUENCE LISTING

The present application is being filed along with a Sequence Listing in electronic format. The Sequence Listing is provided as a file entitled BIOL0405WOSEQ_ST25.txt, created on Sep. 28, 2021, which is 124 Kb in size. The information in the electronic format of the sequence listing is incorporated herein by reference in its entirety.

### FIELD

Provided are compounds and pharmaceutical compositions for reducing the amount or activity of Charged Multivesicular Body Protein 7 (CHMP7) RNA in a cell or subject, and in certain instances reducing the amount of CHMP7 protein in a cell or subject. Such compounds and pharmaceutical compositions are useful to ameliorate diseases or conditions associated with aberrant activation of Endosomal Sorting Complexes Required for Transport-III proteins.

### BACKGROUND

The nuclear envelope has an important role in maintaining the separation between the nucleus and cytoplasm of eukaryotic cells. Defective nuclear envelopes can cause cell death, losses in genome integrity, and disease. These defects can involve either inefficient sealing of the nuclear membrane and/or inappropriate assembly of nuclear pore complexes. (Thaller, D. J., et al., bioRxiv, 2020, 2020.2005.2004.074880, doi:10.1101/2020.05.05.074880).

During eukaryotic cell division, the nuclear envelope is broken down and reformed using a complex process involving Endosomal Sorting Complexes Required for Transport-III (ESCRT-III) proteins. These ESCRT-III proteins have been implicated in sealing holes in the nuclear envelope in mammals and ensuring quality control of nuclear pore complexes (NPCs). Charged Multivesicular Body Protein 7 (CHMP7) is an ESCRTII/III protein that has been implicated in recruiting additional ESCRT-III proteins to holes in the nuclear membrane, and in sealing nuclear pores to protect the compartmentalization of the nucleus and cytoplasm (Gu, M., et al., Proc. Natl. Acad. Sci. 2017, 114, E2166-e2175, doi:10.1073/pnas.1613916114). Studies in yeast using the CHMP7 ortholog Chm7, indicate that controlling CHMP7 activation is critical to prevent the protein taking on gain-of-function roles at the nuclear envelope. Such over-activation of CHMP7 could lead to inappropriate sealing of nuclear membrane pores, and defects in the assembly of the nuclear pore complex (Thaller, D. J., et al., Elife, 2019, 8, doi:10.7554/eLife.45284; Webster, B. M., et al., EMBO 2016, 25, 10.15252/embj.201694574). CHMP7 has been identified as a potential therapeutic target for familial and sporadic amyotrophic lateral sclerosis (ALS), frontotemporal dementia (FTD), and possibly other neurodegenerative diseases associated with nucleoporin reduction and TDP-43 pathology (Coyne, A. N., et al., Science Trans. Med., 2021, 13 (604):eabe1923, doi: 10.1126/scitranslmed.abe1923. PMID: 34321318).

Currently there is a lack of acceptable options for treating diseases associated with over-activation of the ESCRT-III proteins, resulting in inappropriate sealing of pores in the nuclear membrane, defective nuclear envelopes, or defective nuclear pore complexes. It is therefore an object herein to provide compounds and pharmaceutical compositions for the treatment of such diseases.

### SUMMARY OF THE INVENTION

Provided herein are compounds and pharmaceutical compositions for reducing the amount or activity of CHMP7 RNA, and in certain embodiments reducing the amount of CHMP7 protein in a cell or animal. In certain embodiments, the animal has a disease or disorder associated with over-expression of CHMP7. In certain embodiments, the animal has a disease or disorder associated with over-activation of CHMP7 protein activity. In certain embodiments, the animal has a disease or condition associated with defects in the nuclear envelope. In certain embodiments, the defect in the nuclear envelope comprises a defect in nuclear pore closure. In certain embodiments, the animal has a disease or condition associated with a defect in the assembly of the nuclear pore complex. In certain embodiments, compounds are useful for reducing expression of CHMP7 RNA. In certain embodiments, compounds useful for reducing expression of CHMP7 RNA are oligomeric compounds. In certain embodiments, compounds useful for reducing expression of CHMP7 RNA and/or CHMP7 protein are modified oligonucleotides.

### DETAILED DESCRIPTION OF THE INVENTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included” is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, treatises, and GenBank, ENSEMBL, and NCBI reference sequence records, are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

### Definitions

As used herein, “2′-deoxynucleoside” means a nucleoside comprising a 2′-H(H) deoxyfuranosyl sugar moiety. In certain embodiments, a 2′-deoxynucleoside is a 2′-β-D-deoxynucleoside and comprises a 2′-β-D-deoxyribosyl sugar moiety, which has the β-D ribosyl configuration as found in naturally occurring deoxyribonucleic acids (DNA). In certain embodiments, a 2′-deoxynucleoside may comprise a modified nucleobase or may comprise an RNA nucleobase (uracil).

As used herein, “2′-MOE” means a 2′-OCH₂CH₂OCH₃ group in place of the 2′-OH group of a furanosyl sugar moiety. A “2′-MOE modified sugar moiety” means a sugar moiety with a 2′-OCH₂CH₂OCH₃ group in place of the 2′-OH group of a furanosyl sugar moiety. Unless otherwise indicated, a 2′-MOE modified sugar moiety is in the β-D-ribosyl configuration. “MOE” means O-methoxyethyl.

As used herein, “2′-MOE nucleoside” means a nucleoside comprising a 2′-MOE modified sugar moiety.

As used herein, “2′-OMe” means a 2′-OCH₃ group in place of the 2′-OH group of a furanosyl sugar moiety. A “2′-O-methyl modified sugar moiety” or “2′-OMe modified sugar moiety” means a sugar moiety with a 2′-OCH₃ group in place of the 2′-OH group of a furanosyl sugar moiety. Unless otherwise indicated, a 2′-OMe modified sugar moiety is in the β-D-ribosyl configuration.

As used herein, “2′-OMe nucleoside” means a nucleoside comprising a 2′-OMe modified sugar moiety.

As used herein, “2′-substituted nucleoside” means a nucleoside comprising a 2′-substituted sugar moiety. As used herein, “2′-substituted” in reference to a sugar moiety means a sugar moiety comprising at least one 2′-substituent group other than H or OH.

As used herein, “5-methyl cytosine” means a cytosine modified with a methyl group attached to the 5 position. A 5-methyl cytosine is a modified nucleobase.

As used herein, “ameliorate” in reference to a disease or condition means improvement in at least one symptom of the disease or condition relative to the same symptom in the absence of the treatment. In certain embodiments, amelioration is the reduction in the severity or frequency of the symptom or the delayed onset or slowing of progression in the severity or frequency of a symptom.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound.

As used herein, “antisense agent” means an antisense compound and optionally one or more additional features, such as a sense compound.

As used herein, “antisense compound” means an antisense oligonucleotide and optionally one or more additional features, such as a conjugate group. As used herein, “antisense compound” means an oligomeric compound capable of achieving at least one antisense activity.

As used herein, “sense compound” means a sense oligonucleotide and optionally one or more additional features, such as a conjugate group.

As used herein, “antisense oligonucleotide” means an oligonucleotide, including the oligonucleotide portion of an antisense compound, that is capable of hybridizing to a target nucleic acid and is capable of at least one antisense activity Antisense oligonucleotides include but are not limited to antisense RNAi oligonucleotides and antisense RNase H oligonucleotides.

As used herein, “sense oligonucleotide” means an oligonucleotide, including the oligonucleotide portion of a sense compound, that is capable of hybridizing to an antisense oligonucleotide.

As used herein, “bicyclic nucleoside” or “BNA” means a nucleoside comprising a bicyclic sugar moiety.

As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the furanosyl sugar moiety is a ribosyl moiety. In certain embodiments, the bicyclic sugar moiety does not comprise a furanosyl moiety.

As used herein, “cerebrospinal fluid” or “CSF” means the fluid filling the space around the brain and spinal cord. “Artificial cerebrospinal fluid” or “aCSF” means a prepared or manufactured fluid that has certain properties of cerebrospinal fluid.

As used herein, “cleavable moiety” means a bond or group of atoms that is cleaved under physiological conditions, for example, inside a cell, an animal, or a human.

As used herein, “complementary” in reference to an oligonucleotide means that at least 70% of the nucleobases of the oligonucleotide or one or more portions thereof and the nucleobases of another nucleic acid or one or more portions thereof are capable of hydrogen bonding with one another when the nucleobase sequence of the oligonucleotide and the other nucleic acid are aligned in opposing directions. As used herein, “complementary nucleobases” means nucleobases that are capable of forming hydrogen bonds with one another. Complementary nucleobase pairs include adenine (A) and thymine (T), adenine (A) and uracil (U), cytosine (C) and guanine (G), 5-methyl cytosine (^{m}C) and guanine (G). Complementary oligonucleotides and/or target nucleic acids need not have nucleobase complementarity at each nucleoside. Rather, some mismatches are tolerated. As used herein, “fully complementary” or “100% complementary” in reference to an oligonucleotide, or a portion thereof, means that the oligonucleotide, or portion thereof, is complementary to another oligonucleotide or target nucleic acid at each nucleobase of the shorter of the two oligonucleotides, or at each nucleoside if the oligonucleotides are the same length.

As used herein, “conjugate group” means a group of atoms that is directly or indirectly attached to an oligonucleotide. Conjugate groups include a conjugate moiety and a conjugate linker that attaches the conjugate moiety to the oligonucleotide.

As used herein, “conjugate linker” means a single bond or a group of atoms comprising at least one bond that connects a conjugate moiety to an oligonucleotide.

As used herein, “conjugate moiety” means a group of atoms that is attached to an oligonucleotide via a conjugate linker.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “cEt” means a 4′ to 2′ bridge in place of the 2′OH-group of a ribosyl sugar moiety, wherein the bridge has the formula of 4′-CH(CH₃)—O-2′, and wherein the methyl group of the bridge is in the S configuration. A “cEt sugar moiety” is a bicyclic sugar moiety with a 4′ to 2′ bridge in place of the 2′OH-group of a ribosyl sugar moiety, wherein the bridge has the formula of 4′-CH(CH₃)—O-2′, and wherein the methyl group of the bridge is in the S configuration. “cEt” means constrained ethyl.

As used herein, “cEt nucleoside” means a nucleoside comprising a cEt sugar moiety.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are compounds comprising modified oligonucleotides.

As used herein, “chirally controlled” in reference to an internucleoside linkage means chirality at that linkage is enriched for a particular stereochemical configuration.

As used herein, “deoxy region” means a region of 5-12 contiguous nucleotides, wherein at least 70% of the nucleosides are 2′-β-D-deoxynucleosides. In certain embodiments, each nucleoside is selected from a 2′-β-D-deoxynucleoside, a bicyclic nucleoside, and a 2′-substituted nucleoside. In certain embodiments, a deoxy region supports RNase H activity. In certain embodiments, a deoxy region is the gap or internal region of a gapmer.

As used herein, “gapmer” means a modified oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings” or “wing segments.” In certain embodiments, the internal region is a deoxy region. The positions of the internal region or gap refer to the order of the nucleosides of the internal region and are counted starting from the 5′-end of the internal region. Unless otherwise indicated, “gapmer” refers to a sugar motif. In certain embodiments, each nucleoside of the gap is a 2′-β-D-deoxynucleoside. In certain embodiments, the gap comprises one 2′-substituted nucleoside at position 1, 2, 3, 4, or 5 of the gap, and the remainder of the nucleosides of the gap are 2′-β-D-deoxynucleosides. As used herein, the term “MOE gapmer” indicates a gapmer having a gap comprising 2′-β-D-deoxynucleosides and wings comprising 2′-MOE nucleosides. As used herein, the term “mixed wing gapmer” indicates a gapmer having wings comprising modified nucleosides comprising at least two different sugar modifications. Unless otherwise indicated, a gapmer may comprise one or more modified internucleoside linkages and/or modified nucleobases and such modifications do not necessarily follow the gapmer pattern of the sugar modifications.

As used herein, “hotspot region” is a range of nucleobases on a target nucleic acid that is amenable to oligomeric compound-mediated reduction of the amount or activity of the target nucleic acid.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, “internucleoside linkage” means the covalent linkage between contiguous nucleosides in an oligonucleotide. As used herein, “modified internucleoside linkage” means any internucleoside linkage other than a phosphodiester internucleoside linkage. “Phosphorothioate internucleoside linkage or “PS internucleoside linkage” is a modified internucleoside linkage in which one of the non-bridging oxygen atoms of a phosphodiester internucleoside linkage is replaced with a sulfur atom.

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

As used herein, “non-bicyclic modified sugar moiety” means a modified sugar moiety that comprises a modification, such as a substituent, that does not form a bridge between two atoms of the sugar to form a second ring.

As used herein, “mismatch” or “non-complementary” means a nucleobase of a first oligonucleotide that is not complementary with the corresponding nucleobase of a second oligonucleotide or target nucleic acid when the first and second oligonucleotide are aligned.

As used herein, “motif” means the pattern of unmodified and/or modified sugar moieties, nucleobases, and/or internucleoside linkages, in an oligonucleotide.

As used herein, “nucleobase” means an unmodified nucleobase or a modified nucleobase. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one unmodified nucleobase. A “5-methyl cytosine” is a modified nucleobase. A universal base is a modified nucleobase that can pair with any one of the five unmodified nucleobases. As used herein, “nucleobase sequence” means the order of contiguous nucleobases in a target nucleic acid or oligonucleotide independent of any sugar or internucleoside linkage modification.

As used herein, “nucleoside” means a compound, or a fragment of a compound, comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. Modified nucleosides include abasic nucleosides, which lack a nucleobase. “Linked nucleosides” are nucleosides that are connected in a contiguous sequence (i.e., no additional nucleosides are presented between those that are linked).

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound. The term “oligomeric duplex” means a duplex formed by two oligomeric compounds having complementary nucleobase sequences. Each oligomeric compound of an oligomeric duplex may be referred to as a “duplexed oligomeric compound.”

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to a subject. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water, sterile saline, sterile buffer solution or sterile artificial cerebrospinal fluid.

As used herein, “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein, “pharmaceutical composition” means a mixture of substances suitable for administering to a subject. For example, a pharmaceutical composition may comprise an oligomeric compound and a sterile aqueous solution. In certain embodiments, a pharmaceutical composition shows activity in free uptake assay in certain cell lines.

As used herein, “reducing the amount or activity” refers to a reduction or blockade of the transcriptional expression or activity relative to the transcriptional expression or activity in an untreated or control sample and does not necessarily indicate a total elimination of transcriptional expression or activity.

As used herein, “RNA” means an RNA transcript and includes pre-mRNA and mature mRNA unless otherwise specified.

As used herein, “RNAi agent” means an antisense agent that acts, at least in part, through RISC or Ago2 to modulate a target nucleic acid and/or protein encoded by a target nucleic acid. RNAi agents include, but are not limited to double-stranded siRNA, single-stranded RNAi (ssRNAi), and microRNA, including microRNA mimics RNAi agents may comprise conjugate groups and/or terminal groups. In certain embodiments, an RNAi agent modulates the amount and/or activity, of a target nucleic acid. The term RNAi agent excludes antisense agents that act through RNase H.

As used herein, “RNAi compound” means an antisense compound that acts, at least in part, through RISC or Ago2 to modulate a target nucleic acid and/or protein encoded by a target nucleic acid. RNAi compounds include, but are not limited to double-stranded siRNA, single-stranded RNA (ssRNA), and microRNA, including microRNA mimics. In certain embodiments, an RNAi compound modulates the amount, activity, and/or splicing of a target nucleic acid. The term RNAi compound excludes antisense compounds that act through RNase H.

As used herein, “RNase H agent” means an antisense agent that acts through RNase H to modulate a target nucleic acid and/or protein encoded by a target nucleic acid. In certain embodiments, RNase H agents are single-stranded. In certain embodiments, RNase H agents are double-stranded. RNase H compounds may comprise conjugate groups and/or terminal groups. In certain embodiments, an RNase H agent modulates the amount and/or activity of a target nucleic acid. The term RNase H agent excludes antisense agents that act principally through RISC/Ago2.

As used herein, “self-complementary” in reference to an oligonucleotide means an oligonucleotide that at least partially hybridizes to itself.

As used herein, “standard in vitro assay” means the assay described in Example 1 and reasonable variations thereof.

As used herein, “stereorandom chiral center” in the context of a population of molecules of identical molecular formula means a chiral center having a random stereochemical configuration. For example, in a population of molecules comprising a stereorandom chiral center, the number of molecules having the (5) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate internucleoside linkage.

As used herein, “subject” means a human or non-human animal. The terms “subject” and “individual” are used interchangeably. In certain embodiments, the subject is human.

As used herein, “sugar moiety” means an unmodified sugar moiety or a modified sugar moiety. As used herein, “unmodified sugar moiety” means a 2′-OH(H) β-D-ribosyl sugar moiety, as found in RNA (an “unmodified RNA sugar moiety”), or a 2′-H(H) β-D-deoxyribosyl sugar moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate.

As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or target nucleic acids.

As used herein, “target nucleic acid” and “target RNA” mean a nucleic acid that an antisense compound is designed to affect. Target RNA means an RNA transcript and includes pre-mRNA and mature mRNA unless otherwise specified.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

### CERTAIN EMBODIMENTS

The present disclosure provides the following non-limiting numbered embodiments:

Embodiment 1. An oligomeric compound comprising a modified oligonucleotide consisting of 12 to 50 linked nucleosides wherein the nucleobase sequence of the modified oligonucleotide is at least 90% complementary to an equal length portion of a CHMP7 nucleic acid, and wherein the modified oligonucleotide comprises at least one modification selected from a modified sugar moiety and a modified internucleoside linkage.

Embodiment 2. An oligomeric compound comprising a modified oligonucleotide consisting of 12 to 50 linked nucleosides and having a nucleobase sequence comprising at least 12, at least 13, at least 14, at least 15, at least 16, at least 17, at least 18, at least 19, or 20 contiguous nucleobases of any of the nucleobases of SEQ ID NOs: 10-477, wherein the modified oligonucleotide comprises at least one modification selected from a modified sugar moiety and a modified internucleoside linkage.

Embodiment 3. The oligomeric compound of embodiment 2, wherein the modified oligonucleotide has a nucleobase sequence consisting of the nucleobase sequence of any of SEQ ID NOs: 10-477.

Embodiment 4. An oligomeric compound comprising a modified oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19, or at least 20 contiguous nucleobases complementary to:
an equal length portion within nucleobases 3950-3983 of SEQ ID NO: 1;
an equal length portion within nucleobases 4242-4266 of SEQ ID NO: 1;
an equal length portion within nucleobases 4480-4525 of SEQ ID NO: 1;
an equal length portion within nucleobases 4534-4566 of SEQ ID NO: 1;
an equal length portion within nucleobases 5205-5232 of SEQ ID NO: 1;
an equal length portion within nucleobases 5404-5430 of SEQ ID NO: 1;
an equal length portion within nucleobases 8323-8344 of SEQ ID NO: 1;
an equal length portion within nucleobases 16927-16950 of SEQ ID NO: 1;
an equal length portion within nucleobases 17298-17340 of SEQ ID NO: 1; or
an equal length portion within nucleobases 18287-18313 of SEQ ID NO: 1;
wherein the modified oligonucleotide comprises at least one modification selected from a modified sugar moiety and a modified internucleoside linkage.

Embodiment 5. An oligomeric compound comprising a modified oligonucleotide consisting of 12 to 30 linked nucleosides and having a nucleobase sequence comprising at least 8, at least 9, at least 10, at least 11, at least 12, at least 13, at least 14, at least 15, at least 16, at least 17, or at least 18 contiguous nucleobases of a sequence selected from:
SEQ ID NOs: 220, 302, and 345;
SEQ ID NOs: 21, 131, 191, and 465;
SEQ ID NOs: 34, 116, 184, 242, 257, 340, and 474;
SEQ ID NOs: 55, 118, 202, 267, 372, and 422;
SEQ ID NOs: 73, 136, 197, and 421;
SEQ ID NOs: 79, 160, 168, 230, 313, 331, and 464;
SEQ ID NOs: 157, 186, and 265;
SEQ ID NOs: 128, 182, and 309;
SEQ ID NOs: 44, 76, 153, 206, 283, 363, and 416; or
SEQ ID NOs: 85, 121, 189, 300, and 354;
wherein the modified oligonucleotide comprises at least one modification selected from a modified sugar moiety and a modified internucleoside linkage.

Embodiment 6. The oligomeric compound of any of embodiments 1-5, wherein the modified oligonucleotide has a nucleobase sequence that is at least 80%, 85%, 90%, 95%, or 100% complementary to the nucleobase sequence of SEQ ID NO: 1 or SEQ ID NO: 2 when measured across the entire nucleobase sequence of the modified oligonucleotide.

Embodiment 7. The oligomeric compound of any of embodiments 1-6, wherein the modified oligonucleotide comprises at least one modified nucleoside.

Embodiment 8. The oligomeric compound of embodiment 7, wherein the modified oligonucleotide comprises at least one modified nucleoside comprising a modified sugar moiety.

Embodiment 9. The oligomeric compound of embodiment 8, wherein the modified oligonucleotide comprises at least one modified nucleoside comprising a bicyclic sugar moiety.

Embodiment 10. The oligomeric compound of embodiment 9, wherein the modified oligonucleotide comprises at least one modified nucleoside comprising a bicyclic sugar moiety having a 2′-4′ bridge, wherein the 2′-4′ bridge is selected from —O—CH₂—; and —O—CH(CH₃)—.

Embodiment 11. The oligomeric compound of any of embodiments 7-10, wherein the modified oligonucleotide comprises at least one modified nucleoside comprising a non-bicyclic modified sugar moiety.

Embodiment 12. The oligomeric compound of embodiment 11, wherein the modified oligonucleotide comprises at least one modified nucleoside comprising a non-bicyclic modified sugar moiety comprising a 2′-MOE modified sugar moiety or a 2′-OMe modified sugar moiety.

Embodiment 13. The oligomeric compound of any of embodiments 7-12, wherein the modified oligonucleotide comprises at least one modified nucleoside comprising a sugar surrogate.

Embodiment 14. The oligomeric compound of embodiment 13, wherein the sugar surrogate is selected from any of morpholino, modified morpholino, PNA, THP, and F-HNA.

Embodiment 15. The oligomeric compound of any of embodiments 1-8 or 11-14, wherein the modified oligonucleotide does not comprise a bicyclic modified sugar moiety.

Embodiment 16. The oligomeric compound of any of embodiments 1-15, wherein the modified oligonucleotide is a gapmer.

Embodiment 17. The oligomeric compound of any of embodiments 1-16 wherein the modified oligonucleotide comprises a deoxy region consisting of 5-12 linked 2′-deoxynucleosides.

Embodiment 18. The oligomeric compound of any of embodiments 1-16, wherein the modified oligonucleotide comprises a deoxy region consisting of 5-12 linked 2′-β-D-deoxynucleosides.

Embodiment 19. The oligomeric compound of embodiment 17 or embodiment 18, wherein the deoxy region consists of 6, 7, 8, 9, 10, or 6-10 linked nucleosides.

Embodiment 20. The oligomeric compound of any of embodiments 17-19, wherein each nucleoside immediately adjacent to the deoxy region comprises a modified sugar moiety.

Embodiment 21. The oligomeric compound of any of embodiments 17-20, wherein the deoxy region is flanked on the by a 5′-external region consisting of 1-6 linked 5′-external region nucleosides and on the 3′-side by a 3′-external region consisting of 1-6 linked 3′-external region nucleosides; wherein
the 3′-most nucleoside of the 5′ external region comprises a modified sugar moiety; and
the 5′-most nucleoside of the 3′ external region comprises a modified sugar moiety.

Embodiment 22. The oligomeric compound of embodiment 21, wherein each nucleoside of the 3′ external region comprises a modified sugar moiety.

Embodiment 23. The oligomeric compound of embodiment 21 or embodiment 22, wherein each nucleoside of the 5′ external region comprises a modified sugar moiety.

Embodiment 24. The oligomeric compound of any of embodiments 1-23, wherein the modified oligonucleotide comprises:
a 5′-region consisting of 1-7 linked 5′-region nucleosides;
a central region consisting of 6-10 linked central region nucleosides; and
a 3′-region consisting of 1-7 linked 3′-region nucleosides; wherein
each of the 5′-region nucleosides and each of the 3′-region nucleosides comprises a modified sugar moiety and each of the central region nucleosides comprises a 2′-β-D-deoxyfuranosyl sugar moiety.

Embodiment 25. The oligomeric compound of embodiment 24, wherein the modified oligonucleotide comprises:
a 5′-region consisting of 5 linked 5′-region nucleosides;
a central region consisting of 10 linked central region nucleosides; and
a 3′-region consisting of 5 linked 3′-region nucleosides; wherein
each of the 5′-region nucleosides and each of the 3′-region nucleosides is a 2′-MOE nucleoside and each of the central region nucleosides is a 2′-β-D-deoxynucleoside.

Embodiment 26. The oligomeric compound of any of embodiments 1-25, wherein the modified oligonucleotide comprises at least one modified internucleoside linkage.

Embodiment 27. The oligomeric compound of embodiment 26, wherein each internucleoside linkage of the modified oligonucleotide is a modified internucleoside linkage.

Embodiment 28. The oligomeric compound of embodiment 26 or embodiment 27 wherein at least one modified internucleoside linkage is a phosphorothioate internucleoside linkage.

Embodiment 29. The oligomeric compound of embodiment 26 or embodiment 28 wherein the modified oligonucleotide comprises at least one phosphodiester internucleoside linkage.

Embodiment 30. The oligomeric compound of any of embodiments 26, 28, or 29, wherein each internucleoside linkage is either a phosphodiester internucleoside linkage or a phosphorothioate internucleoside linkage.

Embodiment 31. The oligomeric compound of embodiment 27, wherein each modified internucleoside linkage is a phosphorothioate internucleoside linkage Embodiment 32. The oligonucleotide compound of embodiment 26, wherein the modified oligonucleotide has an internucleoside linkage motif of soooossssssssssooss; wherein,
s=a phosphorothioate internucleoside linkage and o=a phosphodiester internucleoside linkage.

Embodiment 33. The oligomeric compound of any of embodiments 1-32, wherein the modified oligonucleotide comprises at least one modified nucleobase.

Embodiment 34. The oligomeric compound of embodiment 33, wherein the modified nucleobase is a 5-methyl cytosine.

Embodiment 35. The oligomeric compound of embodiment 34, wherein each cytosine is a 5-methyl cytosine.

Embodiment 36. The oligomeric compound of any of embodiments 1-35, wherein the modified oligonucleotide consists of 12-30, 12-22, 12-20, 14-18, 14-20, 15-17, 15-25, 16-18, 16-20, 17-20, 18-20 or 18-22 linked nucleosides.

Embodiment 37. The oligomeric compound of any of embodiments 1-36, wherein the modified oligonucleotide consists of 16, 17, 18, 19, or 20 linked nucleosides.

Embodiment 38. The oligomeric compound of any of embodiments 1-35, wherein the modified oligonucleotide consists of 20 linked nucleosides.

Embodiment 39. The oligomeric compound of any of embodiments 1-38, consisting of the modified oligonucleotide.

Embodiment 40. The oligomeric compound of any of embodiments 1-38, wherein the oligomeric compound comprises a conjugate group.

Embodiment 41. The oligomeric compound of embodiment 40, wherein the conjugate group comprises a conjugate moiety and a conjugate linker.

Embodiment 42. The oligomeric compound of embodiment 41, wherein the conjugate linker consists of a single bond.

Embodiment 43. The oligomeric compound of embodiment 41 or embodiment 42, wherein the conjugate linker is cleavable.

Embodiment 44. The oligomeric compound of embodiment 41 or embodiment 43, wherein the conjugate linker comprises 1-3 linker-nucleosides.

Embodiment 45. The oligomeric compound of any of embodiments 41-43, wherein the conjugate linker does not comprise any linker nucleosides.

Embodiment 46. The oligomeric compound of any of embodiments 40-45, wherein the conjugate group is attached to the modified oligonucleotide at the 5′-end of the modified oligonucleotide.

Embodiment 47. The oligomeric compound of any of embodiments 40-45, wherein the conjugate group is attached to the modified oligonucleotide at the 3′-end of the modified oligonucleotide.

Embodiment 48. The oligomeric compound of any of embodiments 40-47, wherein the conjugate group comprises a lipid.

Embodiment 49. The oligomeric compound of any of embodiments 40-47, wherein the conjugate group comprises a cell-targeting moiety.

Embodiment 50. The oligomeric compound of any of embodiments 1-49, further comprising a terminal group.

Embodiment 51. The oligomeric compound of any of embodiments 1-49, wherein the oligomeric compound is a singled-stranded oligomeric compound.

Embodiment 52. The oligomeric compound of any of embodiments 1-51, wherein the oligomeric compound is capable of reducing the amount of CHMP7 RNA in a cell.

Embodiment 53. The oligomeric compound of any of embodiments 1-52, wherein the modified oligonucleotide of the oligomeric compound is a pharmaceutically acceptable salt comprising one or more cations selected from sodium, potassium, calcium, and magnesium.

Embodiment 54. An oligomeric duplex, comprising a first oligomeric compound and a second oligomeric compound comprising a second modified oligonucleotide, wherein the first oligomeric compound is an oligomeric compound of any of embodiments 1-53.

Embodiment 55. An antisense agent comprising an antisense compound, wherein the antisense compound is the oligomeric compound of any of embodiments 1-53 or the oligomeric duplex of embodiment 54.

Embodiment 56. The antisense agent of embodiment 55, wherein the antisense agent is the oligomeric duplex of embodiment 54

Embodiment 57. The antisense agent of embodiment 55 or embodiment 56, wherein the antisense agent is:
an RNase H agent capable of reducing the amount of CHMP7 nucleic acid through the activation of RNase H;
or
an RNAi agent capable of reducing the amount of CHMP7 nucleic acid through the activation of RISC/Ago2.

Embodiment 58. The antisense agent of any of embodiments 55-57, wherein the antisense agent comprises a conjugate group, wherein the conjugate group comprises a cell-targeting moiety.

Embodiment 59. A pharmaceutical composition comprising the oligomeric compound of any of embodiments 1-53, the oligomeric duplex of embodiment 54, or the antisense agent of any of embodiments 55-58, and a pharmaceutically acceptable diluent.

Embodiment 60. The pharmaceutical composition of embodiment 59, wherein the pharmaceutically acceptable diluent is artificial CSF (aCSF) or phosphate-buffered saline (PBS).

Embodiment 61. The pharmaceutical composition of embodiment 60, wherein the pharmaceutical composition consists essentially of the oligomeric compound, oligomeric duplex, or antisense agent, and artificial CSF (aCSF).

Embodiment 62. The pharmaceutical composition of embodiment 60, wherein the pharmaceutical composition consists essentially of the oligomeric compound, oligomeric duplex, or antisense agent, and phosphate buffered saline (PBS).

Embodiment 63. A chirally enriched population of oligomeric compounds of any of embodiments 1-53, wherein the population is enriched for oligomeric compounds comprising at least one particular phosphorothioate internucleoside linkage having a particular stereochemical configuration.

Embodiment 64. The chirally enriched population of embodiment 63, wherein the population is enriched for oligomeric compounds comprising at least one particular phosphorothioate internucleoside linkage having the (Sp) configuration.

Embodiment 65. The chirally enriched population of embodiment 63, wherein the population is enriched for oligomeric compounds comprising at least one particular phosphorothioate internucleoside linkage having the (Rp) configuration.

Embodiment 66. The chirally enriched population of embodiment 63, wherein the population is enriched for oligomeric compounds having a particular, independently selected stereochemical configuration at each phosphorothioate internucleoside linkage.

Embodiment 67. The chirally enriched population of embodiment 66, wherein the population is enriched for oligomeric compounds having the (Sp) configuration at each phosphorothioate internucleoside linkage or for modified oligonucleotides having the (Rp) configuration at each phosphorothioate internucleoside linkage.

Embodiment 68. The chirally enriched population of embodiment 66, wherein the population is enriched for oligomeric compounds having the (Rp) configuration at one particular phosphorothioate internucleoside linkage and the (Sp) configuration at each of the remaining phosphorothioate internucleoside linkages.

Embodiment 69. The chirally enriched population of embodiment 66, wherein the population is enriched for oligomeric compounds having at least 3 contiguous phosphorothioate internucleoside linkages in the Sp, Sp, and Rp configurations, in the 5′ to 3′ direction.

Embodiment 70. A population of oligomeric compounds of any of embodiments 1-53, wherein all of the phosphorothioate internucleoside linkages of the modified oligonucleotide are stereorandom.

Embodiment 71. A pharmaceutical composition comprising the population of oligomeric compounds of any of embodiments 63-70 and a pharmaceutically acceptable diluent.

Embodiment 72. The pharmaceutical composition of embodiment 71, wherein the pharmaceutically acceptable diluent is artificial CSF (aCSF) or phosphate-buffered saline (PBS).

Embodiment 73. The pharmaceutical composition of embodiment 72, wherein the pharmaceutical composition consists essentially of the population of oligomeric compounds and artificial CSF (aCSF).

Embodiment 74. The pharmaceutical composition of embodiment 72, wherein the pharmaceutical composition consists essentially of the population of oligomeric compounds and PBS.

### I. Certain Oligonucleotides

In certain embodiments, provided herein are oligomeric compounds comprising oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage.

A. Certain Modified Nucleosides

Modified nucleosides comprise a modified sugar moiety or a modified nucleobase or both a modified sugar moiety and a modified nucleobase.

1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more substituent groups none of which bridges two atoms of the fumnosyl ring to form a bicyclic structure. Such non bridging substituents may be at any position of the fumnosyl, including but not limited to substituents at the 2′, 4′, and/or 5′ positions. In certain embodiments one or more non-bridging substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂) 2 OCH₃ (“MOE” or “O-methoxyethyl”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugar moieties comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂O N(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCH₃, and OCH₂CH₂OCH₃.

In certain embodiments, modified furanosyl sugar moieties and nucleosides incorporating such modified furanosyl sugar moieties are further defined by isomeric configuration. For example, a 2′-deoxyfuranosyl sugar moiety may be in seven isomeric configurations other than the naturally occurring β-D-deoxyribosyl configuration. Such modified sugar moieties are described in, e.g., WO 2019/157531, incorporated by reference herein. A 2′-modified sugar moiety has an additional stereocenter at the 2′-position relative to a 2′-deoxyfuranosyl sugar moiety; therefore, such sugar moieties have a total of sixteen possible isomeric configurations. 2′-modified sugar moieties described herein are in the β-D-ribosyl isomeric configuration unless otherwise specified.

Certain modified sugar moieties comprise a substituent that bridges two atoms of the furanosyl ring to form a second ring, resulting in a bicyclic sugar moiety. Nucleosides comprising such bicyclic sugar moieties have been referred to as bicyclic nucleosides (BNAs), locked nucleosides, or conformationally restricted nucleotides (CRN). Certain such compounds are described in US Patent Publication No. 2013/0190383; and PCT publication WO 2013/036868. In certain such embodiments, the bicyclic sugar moiety comprises a bridge between the 4′ and the 2′ furanose ring atoms. In certain such embodiments, the furanose ring is a ribose ring. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂, —S(═O)ₓ—, and —N(Rₐ)—;
wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.
   Additional bicyclic sugar moieties are known in the art, see, for example: Freier et al., Nucleic Acids Research, 1997, 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129, 8362-8379; Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; Wengel et al., U.S. Pat. No. 7,053,207, Imanishi et al., U.S. Pat. No. 6,268,490, Imanishi et al. U.S. Pat. No. 6,770,748, Imanishi et al., U.S. Pat. No. RE44,779; Wengel et al., U.S. Pat. No. 6,794,499, Wengel et al., U.S. Pat. No. 6,670,461; Wengel et al., U.S. Pat. No. 7,034,133, Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191, Torsten et al., WO 2004/106356, Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; Allerson et al., US2008/0039618; and Migawa et al., US2015/0191727. In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

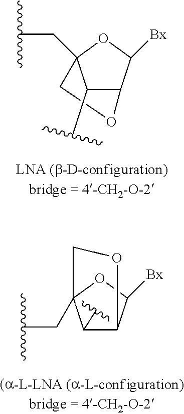

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, C J. Bioorg. & Med. Chem. 2002, 10, 841-854), fluoro HNA:

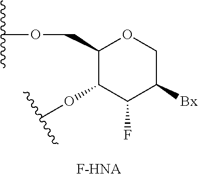

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

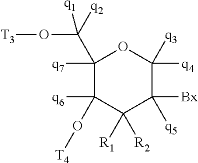

wherein, independently, for each of the modified THP nucleosides:
Bx is a nucleobase moiety;
T₃ and T ₄ are each, independently, an internucleoside linking group linking the modified THP nucleoside to the remainder of an oligonucleotide or one of T ₃ and T ₄ is an internucleoside linking group linking the modified THP nucleoside to the remainder of an oligonucleotide and the other of T ₃ and T ₄ is H, a hydroxyl protecting group, a linked conjugate group, or a 5′ or 3′-terminal group;
q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and
each of R₁ and R ₂ is independently selected from among: hydrogen, halogen, substituted or unsubstituted alkoxy, NJ₁J₂, SJ₁, N₃, OC(═X)J ₁, OC(═X)NJ₁J₂, NJ₃C(═X)NJ₁J₂, and CN, wherein X is O, S or NJ₁, and each J₁, J ₂, and J ₃ is, independently, H or C₁-C₆ alkyl.

In certain embodiments, modified THP nucleosides are provided wherein q₁, q ₂, q ₃, q ₄, q₅, q ₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R ₂ is F. In certain embodiments, R ₁ is F and R ₂ is H, in certain embodiments, R ₁ is methoxy and R ₂ is H, and in certain embodiments, R ₁ is methoxyethoxy and R ₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

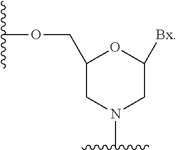

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides.

2. Certain Modified Nucleobases

In certain embodiments, modified oligonucleotides comprise one or more nucleosides comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 5-methyl cytosine, 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp) Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

3. Certain Modified Internucleoside Linkages

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphodiesters, which contain a phosphodiester bond (“P(O₂)═O”) (also referred to as unmodified or naturally occurring linkages), phosphotriesters, methylphosphonates, phosphoramidates, phosphorothioates (“P(O₂)═S”), and phosphorodithioates (“HS-P=S”). Representative non-phosphorus containing internucleoside linking groups include but are not limited to methylenemethylimino (—CH₂—N(CH₃)—O—CH₂-), thiodiester, thionocarbamate (—O—C(═O)(NH)—S—); siloxane (—O—SiH₂—O—); and N,N′-dimethylhydrazine (—CH₂—N(CH₃)—N(CH₃)-). Modified internucleoside linkages, compared to naturally occurring phosphodiester internucleoside linkages, can be used to alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate internucleoside linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate internucleoside linkage. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkage in a particular, independently selected stereochemical configuration. In certain embodiments, the particular configuration of the particular phosphorothioate internucleoside linkage is present in at least 65% of the molecules in the population. In certain embodiments, the particular configuration of the particular phosphorothioate internucleoside linkage is present in at least 70% of the molecules in the population. In certain embodiments, the particular configuration of the particular phosphorothioate internucleoside linkage is present in at least 80% of the molecules in the population. In certain embodiments, the particular configuration of the particular phosphorothioate internucleoside linkage is present in at least 90% of the molecules in the population. In certain embodiments, the particular configuration of the particular phosphorothioate internucleoside linkage is present in at least 99% of the molecules in the population. Such chirally enriched populations of modified oligonucleotides can be generated using synthetic methods known in the art, e.g., methods described in Oka et al., JACS, 2003, 125, 8307, Wan et al., Nuc. Acid. Res., 2014, 42, 13456, and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

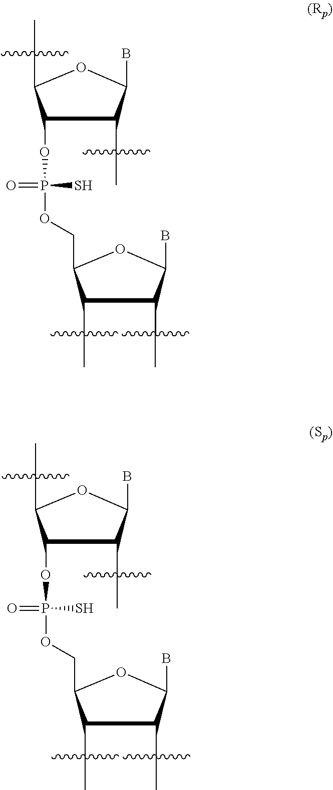

Unless otherwise indicated, chiral internucleoside linkages of modified oligonucleotides described herein can be stereorandom or in a particular stereochemical configuration.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal methoxypropyl (MOP), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (see, for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

B. Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

1. Certain Sugar Motifs

In certain embodiments, oligonucleotides comprise one or more type of modified sugar and/or unmodified sugar moiety arranged along the oligonucleotide or portion thereof in a defined pattern or sugar motif. In certain instances, such sugar motifs include but are not limited to any of the sugar modifications discussed herein.

In certain embodiments, modified oligonucleotides have a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-6 nucleosides. In certain embodiments, each nucleoside of each wing of a gapmer comprises a modified sugar moiety. In certain embodiments, at least one nucleoside of each wing of a gapmer comprises a modified sugar moiety. In certain embodiments, at least two nucleosides of each wing of a gapmer comprises a modified sugar moiety. In certain embodiments, at least three nucleosides of each wing of a gapmer comprises a modified sugar moiety. In certain embodiments, at least four nucleosides of each wing of a gapmer comprises a modified sugar moiety. In certain embodiments, at least five nucleosides of each wing of a gapmer comprises a modified sugar moiety.

In certain embodiments, the gap of a gapmer comprises 7-12 nucleosides. In certain embodiments, each nucleoside of the gap of a gapmer comprises a 2′-deoxyribosyl sugar moiety. In certain embodiments, at least six nucleosides of the gap of a gapmer comprise a 2′-β-D-deoxyribosyl sugar moiety. In certain embodiments, each nucleoside of the gap of a gapmer comprises a 2′-β-D-deoxyribosyl sugar moiety. In certain embodiments, at least one nucleoside of the gap of a gapmer comprises a modified sugar moiety. In certain embodiments, at least one nucleoside of the gap of a gapmer comprises a 2′-OMe sugar moiety.

In certain embodiments, the gapmer is a deoxy gapmer. In certain embodiments, the nucleosides on the gap side of each wing/gap junction comprise 2′-deoxyribosyl sugar moieties and the nucleosides on the wing sides of each wing/gap junction comprise modified sugar moieties. In certain embodiments, at least six nucleosides of the gap of a gapmer comprise a 2′-β-D-deoxyribosyl sugar moiety. In certain embodiments, each nucleoside of the gap of a gapmer comprises a 2′-deoxyribosyl sugar moiety. In certain embodiments, each nucleoside of each wing of a gapmer comprises a modified sugar moiety. In certain embodiments, one nucleoside of the gap comprises a modified sugar moiety and each remaining nucleoside of the gap comprises a 2′-deoxyribosyl sugar moiety.

In certain embodiments, modified oligonucleotides comprise or consist of a portion having a fully modified sugar motif. In such embodiments, each nucleoside of the fully modified portion of the modified oligonucleotide comprises a modified sugar moiety. In certain embodiments, each nucleoside of the entire modified oligonucleotide comprises a modified sugar moiety. In certain embodiments, modified oligonucleotides comprise or consist of a portion having a fully modified sugar motif, wherein each nucleoside within the fully modified portion comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified oligonucleotide comprises the same 2′-modification.

Herein, the lengths (number of nucleosides) of the three regions of a gapmer may be provided using the notation [# of nucleosides in the 5′-wing]-[# of nucleosides in the gap]-[# of nucleosides in the 3′-wing]. Thus, a 5-gapmer consists of 5 linked nucleosides in each wing and 10 linked nucleosides in the gap. Where such nomenclature is followed by a specific modification, that modification is the modification in each sugar moiety of each wing and the gap nucleosides comprises a 2′-β-D-deoxyribosyl sugar moiety. Thus, a 5-10-5 MOE gapmer consists of 5 linked 2′-MOE nucleosides in the 5′-wing, 10 linked a 2′-β-D-deoxynucleosides in the gap, and 5 linked 2′-MOE nucleosides in the 3′-wing. A 3-10-3 cEt gapmer consists of 3 linked cEt nucleosides in the 5′-wing, 10 linked 2′-β-D-deoxynucleosides in the gap, and 3 linked cEt nucleosides in the 3′-wing. A 5-8-5 gapmer consists of 5 linked nucleosides comprising a modified sugar moiety in the 5′-wing, 8 linked a 2′-O-D-deoxynucleosides in the gap, and 5 linked nucleosides comprising a modified sugar moiety in the 3′-wing. A mixed wing gapmer has at least two different modified sugar moieties in the 5′ and/or the 3′ wing. A 5-8-5 or 5-8-4 mixed wing gapmer has at least two different modified sugar moieties in the 5′- and/or the 3′-wing.

In certain embodiments, modified oligonucleotides are 5-10-5 MOE gapmers. In certain embodiments, modified oligonucleotides are 6-10-4 MOE gapmers. In certain embodiments, modified oligonucleotides are 4-10-6 MOE gapmers. In certain embodiments, modified oligonucleotides are 5-8-4 MOE gapmers. In certain embodiments, modified oligonucleotides are 3-10-7 MOE gapmers. In certain embodiments, modified oligonucleotides are 7-10-3 MOE gapmers. In certain embodiments, modified oligonucleotides are 5-8-5 MOE gapmers. In certain embodiments, modified oligonucleotides are 5-9-5 MOE gapmers. In certain embodiments, modified oligonucleotides are X-Y-Z MOE gapmers, wherein X and Z are independently selected from 1, 2, 3, 4, 5, 6, or 7 linked 2′-MOE nucleosides and Y is selected from 7, 8, 9, 10, or 11 linked deoxynucleosides.

In certain embodiments, modified oligonucleotides have the following sugar motif (5′ to 3′): eeeeedyddddddddeeeee, wherein ‘d’ represents a 2′-deoxyribosyl sugar moiety, ‘e’ represents a 2′-MOE sugar moiety, and ‘y’ represents a 2′-OMe sugar moiety.

2. Certain Nucleobase Motifs

In certain embodiments, oligonucleotides comprise modified and/or unmodified nucleobases arranged along the oligonucleotide or portion thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methyl cytosines. In certain embodiments, all of the cytosine nucleobases are 5-methyl cytosines and all of the other nucleobases of the modified oligonucleotide are unmodified nucleobases.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of the nucleoside is a 2′-deoxyribosyl sugar moiety. In certain embodiments, the modified nucleobase is selected from: a 2-thiopyrimidine and a

3. Certain Internucleoside Linkage Motifs

In certain embodiments, oligonucleotides comprise modified and/or unmodified internucleoside linkages arranged along the oligonucleotide or portion thereof in a defined pattern or motif. In certain embodiments, each internucleoside linking group is a phosphodiester internucleoside linkage (P═O). In certain embodiments, each internucleoside linking group of a modified oligonucleotide is a phosphorothioate internucleoside linkage (P═S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate, a (Sp) phosphorothioate, and a (Rp) phosphorothioate. In certain embodiments, the sugar motif of a modified oligonucleotide is a gapmer and the internucleoside linkages within the gap are all modified. In certain such embodiments, some or all of the internucleoside linkages in the wings are unmodified phosphodiester internucleoside linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester internucleoside linkage is not a terminal internucleoside linkage, and the remaining internucleoside linkages are phosphorothioate internucleoside linkages. In certain such embodiments, all of the phosphorothioate internucleoside linkages are stereorandom. In certain embodiments, all of the phosphorothioate internucleoside linkages in the wings are (Sp) phosphorothioates, and the gap comprises at least one Sp, Sp, Rp motif. In certain embodiments, populations of modified oligonucleotides are enriched for modified oligonucleotides comprising such internucleoside linkage motifs.

In certain embodiments, modified oligonucleotides have an internucleoside linkage motif of soooossssssssssooss, wherein each “s” represents a phosphorothioate internucleoside linkage and each “o” represents a phosphodiester internucleoside linkage.

C. Certain Lengths

It is possible to increase or decrease the length of an oligonucleotide without eliminating activity. For example, in Woolf et al., Proc. Natl. Acad. Sci. USA, 1992, 89, 7305-7309, 1992), a series of oligonucleotides 13-25 nucleobases in length were tested for their ability to induce cleavage of a target nucleic acid in an oocyte injection model. Oligonucleotides 25 nucleobases in length with 8 or 11 mismatch bases near the ends of the oligonucleotides were able to direct specific cleavage of the target nucleic acid, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30 or 29 to 30 linked nucleosides.

D. Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Unless otherwise indicated, all modifications are independent of nucleobase sequence.

E. Certain Populations of Modified Oligonucleotides

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population. In certain embodiments, the modified oligonucleotides of a chirally enriched population are enriched for β-D ribosyl sugar moieties, and all of the phosphorothioate internucleoside linkages are stereorandom. In certain embodiments, the modified oligonucleotides of a chirally enriched population are enriched for both β-D ribosyl sugar moieties and at least one, particular phosphorothioate internucleoside linkage in a particular stereochemical configuration.

F. Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a portion of an oligonucleotide has a nucleobase sequence that is complementary to a second oligonucleotide or an identified reference nucleic acid, such as a target nucleic acid. In certain embodiments, the nucleobase sequence of a portion or entire length of an oligonucleotide is at least 50%, at least 60%, at least 70%, at least 80%, at least 85%, at least 90%, at least 95%, or 100% complementary to the second oligonucleotide or nucleic acid, such as a target nucleic acid.

### II. Certain Oligomeric Compounds

In certain embodiments, provided herein are oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, abasic nucleosides, modified or unmodified nucleosides, and two or more nucleosides that are independently modified or unmodified.

A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance.

In certain embodiments, conjugation of one or more carbohydrate moieties to a modified oligonucleotide can optimize one or more properties of the modified oligonucleotide. In certain embodiments, the carbohydrate moiety is attached to a modified subunit of the modified oligonucleotide. For example, the ribose sugar of one or more ribonucleotide subunits of a modified oligonucleotide can be replaced with another moiety, e.g. a non-carbohydrate (preferably cyclic) carrier to which is attached a carbohydrate ligand A ribonucleotide subunit in which the ribose sugar of the subunit has been so replaced is referred to herein as a ribose replacement modification subunit (RRMS), which is a modified sugar moiety. A cyclic carrier may be a carbocyclic ring system, i.e., one or more ring atoms may be a heteroatom, e.g., nitrogen, oxygen, sulphur. The cyclic carrier may be a monocyclic ring system, or may contain two or more rings, e.g. fused rings. The cyclic carrier may be a fully saturated ring system, or it may contain one or more double bonds. In certain embodiments, the modified oligonucleotide is a gapmer.

In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

In certain embodiments, the conjugate group may comprise a conjugate moiety selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl.

In certain embodiments, the conjugate group may comprise a conjugate moiety selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, or C5 alkyl, where the alkyl chain has one or more unsaturated bonds.

In certain embodiments, a conjugate group is a lipid having the following structure:

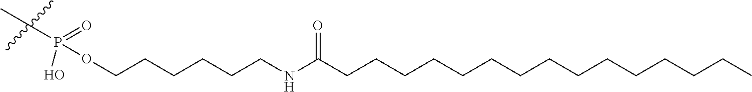 [SMILES: *P(=O)(O)OCCCCCCNC(=O)CCCCCCCCCCCCCCC]

1. Conjugate Moieties

Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, lipophilic groups, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

2. Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-calboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise 1-3 linker nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methyl cytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate or phosphodiester linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxynucleoside that is attached to either the 3′ or 5′-terminal nucleoside of an oligonucleotide by a phosphodiester internucleoside linkage and covalently attached to the remainder of the conjugate linker or conjugate moiety by a phosphate or phosphorothioate internucleoside linkage. In certain such embodiments, the cleavable moiety is 2′-deoxyadenosine.

3. Cell-Targeting Moieties

In certain embodiments, a conjugate group comprises a cell-targeting moiety. In certain embodiments, a conjugate group has the general formula:

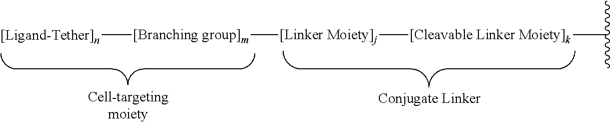

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, each ligand of a cell-targeting moiety has an affinity for at least one type of receptor on a target cell. In certain embodiments, each ligand has an affinity for at least one type of receptor on the surface of a mammalian liver cell. In certain embodiments, each ligand has an affinity for the hepatic asialoglycoprotein receptor (ASGP-R). In certain embodiments, each ligand is a carbohydrate.

In certain embodiments, a conjugate group comprises a cell-targeting conjugate moiety. In certain embodiments, a conjugate group has the general formula:

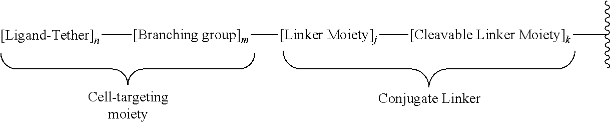

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

B. Certain Terminal Groups

In certain embodiments, oligomeric compounds comprise one or more terminal groups. In certain such embodiments, oligomeric compounds comprise a stabilized 5′-phosphate. Stabilized 5′-phosphates include, but are not limited to 5′-phosphonates, including, but not limited to 5′-vinylphosphonates. In certain embodiments, terminal groups comprise one or more abasic nucleosides and/or inverted nucleosides. In certain embodiments, terminal groups comprise one or more 2′-linked nucleosides. In certain such embodiments, the 2′-linked nucleoside is an abasic nucleoside.

### III. Oligomeric Duplexes

In certain embodiments, oligomeric compounds described herein comprise an oligonucleotide, having a nucleobase sequence complementary to that of a target nucleic acid. In certain embodiments, an oligomeric compound is paired with a second oligomeric compound to form an oligomeric duplex. Such oligomeric duplexes comprise a first oligomeric compound having a portion complementary to a target nucleic acid and a second oligomeric compound having a portion complementary to the first oligomeric compound. In certain embodiments, the first oligomeric compound of an oligomeric duplex comprises or consists of (1) a modified or unmodified oligonucleotide and optionally a conjugate group and (2) a second modified or unmodified oligonucleotide and optionally a conjugate group. Either or both oligomeric compounds of an oligomeric duplex may comprise a conjugate group. The oligonucleotides of each oligomeric compound of an oligomeric duplex may include non-complementary overhanging nucleosides.

### Certain Oligomeric Duplexes

Certain embodiments are directed to oligomeric duplexes comprising a first oligomeric compound and a second oligomeric compound.

In certain embodiments, an oligomeric duplex comprises:
a first oligomeric compound comprising a first modified oligonucleotide consisting of 12 to 50 linked nucleosides and having a nucleobase sequence comprising at least 12, 13, 14, 15, 16, 17, 18, 19, or 20 contiguous nucleobases of any of SEQ ID NOs: 10-477; and
a second oligomeric compound comprising a second modified oligonucleotide consisting of 12 to 50 linked nucleosides wherein the nucleobase sequence of the second modified oligonucleotide comprises a complementary region of at least 8 nucleobases that is at least 90% complementary to an equal length portion of the first modified oligonucleotide. In certain embodiments, the nucleobase sequence of the first modified oligonucleotide is at least 85%, at least 90%, at least 95%, or 100% complementary to an equal length portion of the CHMP7 nucleic acid.

In certain embodiments, an oligomeric duplex comprises:
a first oligomeric compound comprising a first modified oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19, or at least 20 contiguous nucleobases complementary to an equal length portion within nucleobases 3950-3983, 4242-4266, 4480-4525, 4534-4566, 5205-5232, 5404-5430, 8323-8344, 16927-16950, 17298-17340, or 18287-18313 of SEQ ID NO: 1; and
a second oligomeric compound comprising a second modified oligonucleotide consisting of 12 to 30 linked nucleosides wherein the nucleobase sequence of the second modified oligonucleotide comprises a complementary region of at least 8 nucleobases that is at least 90% complementary to an equal length portion of the first modified oligonucleotide. In certain embodiments, the nucleobase sequence of the first modified oligonucleotide is at least 85%, at least 90%, at least 95%, or 100% complementary to an equal length portion of the CHMP7 nucleic acid.

In certain embodiments, an oligomeric duplex comprises:
a first oligomeric compound comprising a first modified oligonucleotide consisting of 12 to 50 linked nucleosides and having a nucleobase sequence comprising at least 12, 13, 14, 15, 16, 17, 18, 19, or 20 contiguous nucleobases of any of SEQ ID NOs: 10-477; wherein each thymine is replaced by uracil; and
a second oligomeric compound comprising a second modified oligonucleotide consisting of 12 to 50 linked nucleosides wherein the nucleobase sequence of the second modified oligonucleotide comprises a complementary region of at least 8 nucleobases that is at least 90% complementary to an equal length portion of the first modified oligonucleotide. In certain embodiments, the nucleobase sequence of the first modified oligonucleotide is at least 85%, at least 90% In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide.

In certain embodiments, an oligomeric duplex comprises:
a first oligomeric compound comprising a first modified oligonucleotide consisting of 16 to 50 linked nucleosides wherein the nucleobase sequence of the first modified oligonucleotide comprises the nucleobase sequence of any of SEQ ID NOs 10-477, wherein each thymine is replaced by uracil; and
a second oligomeric compound comprising a second modified oligonucleotide consisting of 16 to 50 linked nucleosides wherein the nucleobase sequence of the second modified oligonucleotide comprises a complementary region of at least 16 nucleobases that is at least 90% complementary to an equal length portion of the first modified oligonucleotide.

In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide.

In any of the oligomeric duplexes described herein, at least one nucleoside of the first modified oligonucleotide and/or the second modified oligonucleotide can comprise a modified sugar moiety. Examples of suitable modified sugar moieties include, but are not limited to, a bicyclic sugar moiety, such as a 2′-4′ bridge selected from —O—CH2-; and —O—CH(CH3)-, and a non-bicyclic sugar moiety, such as a 2′-MOE modified sugar moiety, a 2′-F modified sugar moiety, or a 2′-OMe modified sugar moiety. In certain embodiments, at least 80%, at least 90%, or 100% of the nucleosides of the first modified oligonucleotide and/or the second modified oligonucleotide comprises a modified sugar moiety selected from 2′-F and 2′-OMe.

In any of the oligomeric duplexes described herein, at least one nucleoside of the first modified oligonucleotide and/or the second modified oligonucleotide can comprise a sugar surrogate. Examples of suitable sugar surrogates include, but are not limited to, morpholino, peptide nucleic acid (PNA), glycol nucleic acid (GNA), and unlocked nucleic acid (UNA). In certain embodiments, at least one nucleoside of the first modified oligonucleotide comprises a sugar surrogate, which can be a GNA.

In any of the oligomeric duplexes described herein, at least one internucleoside linkage of the first modified oligonucleotide and/or the second modified oligonucleotide can comprise a modified internucleoside linkage. In certain embodiments, the modified internucleoside linkage is a phosphorothioate internucleoside linkage. In certain embodiments, at least one of the first, second, or third internucleoside linkages from the 5′ end and/or the 3′ end of the first modified oligonucleotide comprises a phosphorothioate linkage. In certain embodiments, at least one of the first, second, or third internucleoside linkages from the 5′ end and/or the 3′ end of the second modified oligonucleotide comprises a phosphorothioate linkage.

In any of the oligomeric duplexes described herein, at least one internucleoside linkage of the first modified oligonucleotide and/or the second modified oligonucleotide can comprise a phosphodiester internucleoside linkage.

In any of the oligomeric duplexes described herein, each internucleoside linkage of the first modified oligonucleotide and/or the second modified oligonucleotide can be independently selected from a phosphodiester or a phosphorothioate internucleoside linkage.

In any of the oligomeric duplexes described herein, at least one nucleobase of the first modified oligonucleotide and/or the second modified oligonucleotide can be modified nucleobase. In certain embodiments, the modified nucleobase is 5-methylcytosine.

In any of the oligomeric duplexes described herein, the first modified oligonucleotide can comprise a stabilized phosphate group attached to the 5′ position of the 5′-most nucleoside. In certain embodiments, the stabilized phosphate group comprises a cyclopropyl phosphonate or an (E)-vinyl phosphonate.

In any of the oligomeric duplexes described herein, the first modified oligonucleotide can comprise a conjugate group. In certain embodiments, the conjugate group comprises a conjugate linker and a conjugate moiety. In certain embodiments, the conjugate group is attached to the first modified oligonucleotide at the 5′-end of the first modified oligonucleotide. In certain embodiments, the conjugate group is attached to the first modified oligonucleotide at the 3′-end of the modified oligonucleotide. In certain embodiments, the conjugate group comprises N-acetyl galactosamine. In certain embodiments, the conjugate group comprises a cell-targeting moiety having an affinity for transferrin receptor (TfR), also known as TfR1 and CD71. In certain embodiments, the conjugate group comprises an anti-TfR1 antibody or fragment thereof. In certain embodiments, the conjugate group comprises a protein or peptide capable of binding TfR1. In certain embodiments, the conjugate group comprises an aptamer capable of binding TfR1. In certain embodiments, the conjugate group may comprise a conjugate moiety selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl. In certain embodiments, the conjugate group may comprise a conjugate moiety selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, or C5 alkyl, where the alkyl chain has one or more unsaturated bonds.

In any of the oligomeric duplexes described herein, the second modified oligonucleotide can comprise a conjugate group. In certain embodiments, the conjugate group comprises a conjugate linker and a conjugate moiety. In certain embodiments, the conjugate group is attached to the second modified oligonucleotide at the 5′-end of the second modified oligonucleotide. In certain embodiments, the conjugate group is attached to the second modified oligonucleotide at the 3′-end of the modified oligonucleotide. In certain embodiments, the conjugate group comprises N-acetyl galactosamine. In certain embodiments, the conjugate group comprises a cell-targeting moiety having an affinity for transferrin receptor (TfR), also known as TfR1 and CD71. In certain embodiments, the conjugate group comprises an anti-TfR1 antibody or fragment thereof. In certain embodiments, the conjugate group comprises a protein or peptide capable of binding TfR1. In certain embodiments, the conjugate group comprises an aptamer capable of binding TfR1. In certain embodiments, the conjugate group may comprise a conjugate moiety selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl. In certain embodiments, the conjugate group may comprise a conjugate moiety selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, or C5 alkyl, where the alkyl chain has one or more unsaturated bonds.

In certain embodiments, an antisense agent comprises an antisense compound, which comprises an oligomeric compound or an oligomeric duplex described herein. In certain embodiments, an antisense agent, which can comprise an oligomeric compound or an oligomeric duplex described herein, is an RNAi agent capable of reducing the amount of CHMP7 nucleic acid through the activation of RISC/Ago2.

Certain embodiments provide an oligomeric agent comprising two or more oligomeric duplexes. In certain embodiments, an oligomeric agent comprises two or more of any of the oligomeric duplexes described herein. In certain embodiments, an oligomeric agent comprises two or more of the same oligomeric duplex, which can be any of the oligomeric duplexes described herein. In certain embodiments, the two or more oligomeric duplexes are linked together. In certain embodiments, the two or more oligomeric duplexes are covalently linked together. In certain embodiments, the second modified oligonucleotides of two or more oligomeric duplexes are covalently linked together. In certain embodiments, the second modified oligonucleotides of two or more oligomeric duplexes are covalently linked together at their 3′ ends. In certain embodiments, the two or more oligomeric duplexes are covalently linked together by a glycol linker, such as a tetraethylene glycol linker. Certain such compounds are described in, e.g., Alterman, et al., Nature Biotech., 37:844-894, 2019, at least 95%, or 100% complementary to an equal length portion of the CHMP7 nucleic acid.

### IV. Antisense Activity

In certain embodiments, oligomeric compounds and oligomeric duplexes are capable of hybridizing to a target nucleic acid, resulting in at least one antisense activity; such oligomeric compounds and oligomeric duplexes are antisense compounds. In certain embodiments, antisense compounds have antisense activity when they reduce the amount or activity of a target nucleic acid by 25% or more in the standard in vitro assay. In certain embodiments, antisense compounds selectively affect one or more target nucleic acid. Such antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, described herein are antisense compounds that are sufficiently “DNA-like” to elicit RNase H activity. In certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute Antisense compounds that are loaded into RISC are RNAi compounds. RNAi compounds may be double-stranded (siRNA) or single-stranded (ssRNA).

In certain embodiments, hybridization of an antisense compound to a target nucleic acid does not result in recruitment of a protein that cleaves that target nucleic acid. In certain embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain embodiments, hybridization of an antisense compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein and/or a phenotypic change in a cell or subject.

### V. Certain Target Nucleic Acids

In certain embodiments, oligomeric compounds comprise or consist of an oligonucleotide comprising a portion that is complementary to a target nucleic acid. In certain embodiments, the target nucleic acid is an endogenous RNA molecule. In certain embodiments, the target nucleic acid encodes a protein. In certain such embodiments, the target nucleic acid is selected from: a mature mRNA and a pre-mRNA, including intronic, exonic and untranslated regions. In certain embodiments, the target nucleic acid is a mature mRNA. In certain embodiments, the target nucleic acid is a pre-mRNA. In certain embodiments, the target region is entirely within an intron. In certain embodiments, the target region spans an intron/exon junction. In certain embodiments, the target region is at least 50% within an intron.

A. Complementarity/Mismatches to the Target Nucleic Acid

It is possible to introduce mismatch bases without eliminating activity. For example, Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo. Maher and Dolnick (Nuc. Acid. Res. 16:3341-3358, 1988) tested a series of tandem 14 nucleobase oligonucleotides, and a 28 and 42 nucleobase oligonucleotides comprised of the sequence of two or three of the tandem oligonucleotides, respectively, for their ability to arrest translation of human DHFR in a rabbit reticulocyte assay. Each of the three 14 nucleobase oligonucleotides alone was able to inhibit translation, albeit at a more modest level than the 28 or 42 nucleobase oligonucleotides.

In certain embodiments, oligonucleotides are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a portion that is 100% or fully complementary to a target nucleic acid. In certain embodiments, the portion of full complementarity is 6, 7, 8, 9, 10, 11, 12, 13, 14, 15, 16, 17, 18, 19, 20, 21, 22, 23, or 24 nucleobases in length.

In certain embodiments, oligonucleotides comprise one or more mismatched nucleobases relative to the target nucleic acid. In certain embodiments, antisense activity against the target is reduced by such mismatch, but activity against a non-target is reduced by a greater amount. Thus, in certain embodiments selectivity of the oligonucleotide is improved. In certain embodiments, the mismatch is specifically positioned within an oligonucleotide having a gapmer motif. In certain embodiments, the mismatch is at position 1, 2, 3, 4, 5, 6, 7, 8, 9, 10, or 11 from the 5′-end of the gap region. In certain embodiments, the mismatch is at position 1, 2, 3, 4, 5, or 6 from the 5′-end of the 5′ wing region or the 3′ wing region.

B. CHMP7

In certain embodiments, oligomeric compounds comprise or consist of an oligonucleotide that is complementary to a target nucleic acid, wherein the target nucleic acid is a CHMP7 nucleic acid. In certain embodiments, the CHMP7 nucleic acid has the nucleobase sequence set forth in SEQ ID NO: 1 (ENSEMBLGene ID ENSG00000147457.14 from ENSEMBL Release 101: August 2020) or SEQ ID NO: 2 (GENBANK Accession No. NM_152272.5).

In certain embodiments, contacting a cell with an oligomeric compound complementary to SEQ ID NO: 1 or SEQ ID NO: 2 reduces the amount of CHMP7 RNA, and in certain embodiments reduces the amount of CHMP7 protein. In certain embodiments, contacting a cell with an oligomeric compound complementary to SEQ ID NO: 1 or SEQ ID NO: 2 reduces the amount of CHMP7 RNA in a cell, and in certain embodiments reduces the amount of CHMP7 protein in a cell. In certain embodiments, the cell is in vitro. In certain embodiments, the oligomeric compound consists of a modified oligonucleotide.

In certain embodiments, an oligomeric compound complementary to SEQ ID NO: 1 or SEQ ID NO: 2 is capable of reducing the detectable amount of CHMP7 RNA in vitro by at least 10%, at least 20%, at least 30%, at least 40%, at least 50%, at least 60%, at least 70%, at least 80%, or at least 90% in the standard in vitro assay. In certain embodiments, an oligomeric compound complementary to SEQ ID NO: 1 or SEQ ID NO: 2 is capable of reducing the detectable amount of CHMP7 protein in vitro by at least 10%, at least 20%, at least 30%, at least 40%, at least 50%, at least 60%, at least 70%, at least 80%, or at least 90% in the standard in vitro assay. In certain embodiments, an oligomeric compound complementary to SEQ ID NO: 1 or SEQ ID NO: 2 is capable of reducing the detectable amount of CHMP7 RNA in vivo by at least 10%, at least 20%, at least 30%, at least 40%, at least 50%, at least 60%, at least 70%, at least 80%, or at least 90%. In certain embodiments, an oligomeric compound complementary to SEQ ID NO: 1 or SEQ ID NO: 2 is capable of reducing the detectable amount of CHMP7 protein in vivo by at least 10%, at least 20%, at least 30%, at least 40%, at least 50%, at least 60%, at least 70%, at least 80%, or at least 90%. In certain embodiments, an oligomeric compound complementary to SEQ ID NO: 1 or SEQ ID NO: 2, is capable of reducing the detectable amount of CHMP7 RNA in the CSF of a subject by at least 10%, at least 20%, at least 30%, at least 40%, at least 50%, at least 60%, at least 70%, at least 80%, or at least 90%. In certain embodiments, an oligomeric compound complementary to SEQ ID NO: 1 or SEQ ID NO: 2, is capable of reducing the detectable amount of CHMP7 protein in the CSF of a subject by at least 10%, at least 20%, at least 30%, at least 40%, at least 50%, at least 60%, at least 70%, at least 80%, or at least 90%.

### VI. Certain Pharmaceutical Compositions

In certain embodiments, described herein are pharmaceutical compositions comprising one or more oligomeric compounds. In certain embodiments, the one or more oligomeric compounds each consists of a modified oligonucleotide. In certain embodiments, the pharmaceutical composition comprises a pharmaceutically acceptable diluent or carrier. In certain embodiments, a pharmaceutical composition comprises or consists of a sterile saline solution and one or more oligomeric compound. In certain embodiments, the sterile saline is pharmaceutical grade saline. In certain embodiments, a pharmaceutical composition comprises or consists of one or more oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, the sterile PBS is pharmaceutical grade PBS. In certain embodiments, a pharmaceutical composition comprises or consists of one or more oligomeric compound and artificial cerebrospinal fluid (“artificial CSF” or “aCSF”). In certain embodiments, the artificial cerebrospinal fluid is pharmaceutical grade.

In certain embodiments, a pharmaceutical composition comprises a modified oligonucleotide and artificial cerebrospinal fluid (aCSF). In certain embodiments, a pharmaceutical composition consists of a modified oligonucleotide and artificial cerebrospinal fluid. In certain embodiments, a pharmaceutical composition consists essentially of a modified oligonucleotide and artificial cerebrospinal fluid. In certain embodiments, the artificial cerebrospinal fluid is pharmaceutical grade.

In certain embodiments, aCSF comprises sodium chloride, potassium chloride, sodium dihydrogen phosphate dihydrate, sodium phosphate dibasic anhydrous, calcium chloride dihydrate, and magnesium chloride hexahydrate. In certain embodiments, the pH of an aCSF solution is modulated with a suitable pH-adjusting agent, for example, with acids such as hydrochloric acid and alkalis such as sodium hydroxide, to a range of from about 7.1-7.3, or to about 7.2.

In certain embodiments, pharmaceutical compositions comprise one or more oligomeric compound and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric compound encompass any pharmaceutically acceptable salts of the oligomeric compound, esters of the oligomeric compound, or salts of such esters. In certain embodiments, pharmaceutical compositions comprising oligomeric compounds comprising one or more oligonucleotide, upon administration to a subject, including a human, are capable of providing (directly or indirectly) the biologically active metabolite or residue thereof. Accordingly, for example, the disclosure is also drawn to pharmaceutically acceptable salts of oligomeric compounds, prodrugs, pharmaceutically acceptable salts of such prodrugs, and other bioequivalents. In certain embodiments, pharmaceutically acceptable salts comprise inorganic salts, such as monovalent or divalent inorganic salts. Suitable pharmaceutically acceptable salts include, but are not limited to, sodium and, potassium, calcium, and magnesium salts. In certain embodiments, prodrugs comprise one or more conjugate group attached to an oligonucleotide, wherein the conjugate group is cleaved by endogenous nucleases within the body.

In certain embodiments, oligomeric compounds are lyophilized and isolated as sodium salts. In certain embodiments, the sodium salt of an oligomeric compound is mixed with a pharmaceutically acceptable diluent. In certain embodiments, the pharmaceutically acceptable diluent comprises sterile saline, sterile water, PBS, or aCSF. In certain embodiments, the sodium salt of an oligomeric compound is mixed with PBS. In certain embodiments, the sodium salt of an oligomeric compound is mixed with aCSF.

Lipid moieties have been used in nucleic acid therapies in a variety of methods. In certain such methods, the nucleic acid, such as an oligomeric compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents comprising an oligomeric compound provided herein to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), intraneural, perineural, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Under certain conditions, certain compounds disclosed herein act as acids. Although such compounds may be drawn or described in protonated (free acid) form, or ionized and in association with a cation (salt) form, aqueous solutions of such compounds exist in equilibrium among such forms. For example, a phosphate linkage of an oligonucleotide in aqueous solution exists in equilibrium among free acid, anion and salt forms. Unless otherwise indicated, compounds described herein are intended to include all such forms. Moreover, certain oligonucleotides have several such linkages, each of which is in equilibrium. Thus, oligonucleotides in solution exist in an ensemble of forms at multiple positions all at equilibrium. The term “oligonucleotide” is intended to include all such forms. Drawn structures necessarily depict a single form. Nevertheless, unless otherwise indicated, such drawings are likewise intended to include corresponding forms. Herein, a structure depicting the free acid of a compound followed by the term “or salt thereof” or “or a pharmaceutically acceptable salt thereof” expressly includes all such forms that may be fully or partially protonated/de-protonated/in association with a cation or a combination of cations. In certain embodiments, one or more specific cation is identified. The cations include, but are not limited to, sodium, potassium, calcium, and magnesium. In certain embodiments, a structure depicting the free acid of a compound followed by the term “or a pharmaceutically acceptable salt thereof” expressly includes all such forms that may be fully or partially protonated/de-protonated/in association with one or more cations selected from sodium, potassium, calcium, and magnesium.

In certain embodiments, modified oligonucleotides or oligomeric compounds are in aqueous solution with sodium. In certain embodiments, modified oligonucleotides or oligomeric compounds are in aqueous solution with potassium. In certain embodiments, modified oligonucleotides or oligomeric compounds are in PBS. In certain embodiments, modified oligonucleotides or oligomeric compounds are in water. In certain such embodiments, the pH of the solution is adjusted with NaOH and/or HCl to achieve a desired pH.

### VII. Certain Hotspot Regions

In certain embodiments, nucleobases in the ranges specified below comprise a hotspot region of CHMP7 nucleic acid. In certain embodiments, modified oligonucleotides that are complementary to a hotspot region of CHMP7 nucleic acid achieve an average of more than 60% reduction of CHMP7 RNA in the standard in vitro assay

1. Nucleobases 3950-3983 of SEQ ID NO: 1

In certain embodiments, nucleobases 3950-3983 of SEQ ID NO: 1 comprise a hotspot region. In certain embodiments, modified oligonucleotides are complementary to a portion within nucleobases 3950-3983 of SEQ ID NO: 1. In certain embodiments, modified oligonucleotides are 20 nucleobases in length. In certain embodiments, modified oligonucleotides are gapmers. In certain embodiments, the gapmers are MOE gapmers. In certain embodiments, all of the internucleoside linkages of the modified oligonucleotides are phosphorothioate internucleoside linkages. In certain embodiments, the internucleoside linkages of the modified oligonucleotides are phosphorothioate internucleoside linkages and phosphodiester internucleoside linkages. In certain embodiments, the phosphodiester (“o”) and phosphorothioate (“s”) internucleoside linkages are arranged in the order from 5′ to 3′: soooossssssssssooss, wherein each “s” represents a phosphorothioate internucleoside linkage and each “o” represents a phosphodiester internucleoside linkage.

The nucleobase sequences of SEQ ID NOs: 220, 302, and 345 are complementary to a portion of nucleobases 3950-3983 of SEQ ID NO: 1.

The nucleobase sequence of Compound Nos.: 1447312, 1447488, and 1447549 are complementary to a portion within nucleobases 3950-3983 of SEQ ID NO: 1.

In certain embodiments, modified oligonucleotides complementary to a portion within nucleobases 3950-3983 of SEQ ID NO: 1 achieve at least 84% reduction of CHMP7 RNA in the standard in vitro assay. In certain embodiments, modified oligonucleotides complementary to a portion within nucleobases 3950-3983 of SEQ ID NO: 1 achieve an average of 88.7% reduction of CHMP7 RNA in the standard in vitro assay.

2. Nucleobases 4242-4266 of SEQ ID NO: 1

In certain embodiments, nucleobases 4242-4266 of SEQ ID NO: 1 comprise a hotspot region. In certain embodiments, modified oligonucleotides are complementary to a portion within nucleobases 4242-4266 of SEQ ID NO: 1. In certain embodiments, modified oligonucleotides are 20 nucleobases in length. In certain embodiments, modified oligonucleotides are gapmers. In certain embodiments, the gapmers are MOE gapmers. In certain embodiments, all of the internucleoside linkages of the modified oligonucleotides are phosphorothioate internucleoside linkages. In certain embodiments, the internucleoside linkages of the modified oligonucleotides are phosphorothioate internucleoside linkages and phosphodiester internucleoside linkages. In certain embodiments, the phosphodiester (“o”) and phosphorothioate (“s”) internucleoside linkages are arranged in the order from 5′ to 3′: soooossssssssssooss, wherein each “s” represents a phosphorothioate internucleoside linkage and each “o” represents a phosphodiester internucleoside linkage.

The nucleobase sequences of SEQ ID NOs: 21, 131, 191, and 465 are complementary to a portion within nucleobases 4242-4266 of SEQ ID NO: 1.

The nucleobase sequence of Compound Nos.: 1447338, 1447449, 1447242, and 1447606 are complementary to a portion within nucleobases 4242-4266 of SEQ ID NO: 1.

In certain embodiments, modified oligonucleotides complementary to a portion within nucleobases 4242-4266 of SEQ ID NO: 1 achieve at least 60% reduction of CHMP7 RNA in the standard in vitro assay. In certain embodiments, modified oligonucleotides complementary to a portion within nucleobases 4242-4266 of SEQ ID NO: 1 achieve an average of 68% reduction of CHMP7 RNA in the standard in vitro assay.

3. Nucleobases 4480-4525 of SEQ ID NO: 1

In certain embodiments, nucleobases 4480-4525 of SEQ ID NO: 1 comprise a hotspot region. In certain embodiments, modified oligonucleotides are complementary to a portion within nucleobases 4480-4525 of SEQ ID NO: 1. In certain embodiments, modified oligonucleotides are 20 nucleobases in length. In certain embodiments, modified oligonucleotides are gapmers. In certain embodiments, the gapmers are MOE gapmers. In certain embodiments, all of the internucleoside linkages of the modified oligonucleotides are phosphorothioate internucleoside linkages. In certain embodiments, the internucleoside linkages of the modified oligonucleotides are phosphorothioate internucleoside linkages and phosphodiester internucleoside linkages. In certain embodiments, the phosphodiester (“o”) and phosphorothioate (“s”) internucleoside linkages are arranged in the order from 5′ to 3′: soooossssssssssooss, wherein each “s” represents a phosphorothioate internucleoside linkage and each “o” represents a phosphodiester internucleoside linkage.

The nucleobase sequences of SEQ ID NOs: 34, 116, 184, 242, 257, 340, and 474 are complementary to a portion within 4480-4525 of SEQ ID NO: 1.

The nucleobase sequence of Compound Nos.: 1447297, 1447361, 1447311, 1447634, 1447299, 1447279, and 1447636 are complementary to a portion within nucleobases 4480-4525 of SEQ ID NO: 1.

In certain embodiments, modified oligonucleotides complementary to a portion within nucleobases 4480-4525 of SEQ ID NO: 1 achieve at least 41% reduction of CHMP7 RNA in the standard in vitro assay. In certain embodiments, modified oligonucleotides complementary to a portion within nucleobases 4480-4525 of SEQ ID NO: 1 achieve an average of 61% reduction of CHMP7 RNA in the standard in vitro assay.

4. Nucleobases 4534-4566 of SEQ ID NO: 1

In certain embodiments, nucleobases 4534-4566 of SEQ ID NO: 1 comprise a hotspot region. In certain embodiments, modified oligonucleotides are complementary to a portion within nucleobases 4534-4566 of SEQ ID NO: 1. In certain embodiments, modified oligonucleotides are 20 nucleobases in length. In certain embodiments, modified oligonucleotides are gapmers. In certain embodiments, the gapmers are MOE gapmers. In certain embodiments, all of the internucleoside linkages of the modified oligonucleotides are phosphorothioate internucleoside linkages. In certain embodiments, the internucleoside linkages of the modified oligonucleotides are phosphorothioate internucleoside linkages and phosphodiester internucleoside linkages. In certain embodiments, the phosphodiester (“o”) and phosphorothioate (“s”) internucleoside linkages are arranged in the order from 5′ to 3′: soooossssssssssooss, wherein each “s” represents a phosphorothioate internucleoside linkage and each “o” represents a phosphodiester internucleoside linkage.

The nucleobase sequences of SEQ ID NOs: 55, 118, 202, 267, 372, and 422 are complementary to a portion within nucleobases 4534-4566 of SEQ ID NO: 1.

The nucleobase sequence of Compound Nos.: 1447461, 1447313, 1447507, 1447343, 1447400, and 1447387 are complementary to a portion within nucleobases 4534-4566 of SEQ ID NO: 1.

In certain embodiments, modified oligonucleotides complementary to a portion within nucleobases 4534-4566 of SEQ ID NO: 1 achieve at least 58% reduction of CHMP7 RNA in the standard in vitro assay. In certain embodiments, modified oligonucleotides complementary to a portion within nucleobases 4534-4566 of SEQ ID NO: 1 achieve an average of 66% reduction of CHMP7 RNA in the standard in vitro assay.

5. Nucleobases 5205-5232 of SEQ ID NO: 1

In certain embodiments, nucleobases 5205-5232 of SEQ ID NO: 1 comprise a hotspot region. In certain embodiments, modified oligonucleotides are complementary to a portion within nucleobases 5205-5232 of SEQ ID NO: 1. In certain embodiments, modified oligonucleotides are 20 nucleobases in length. In certain embodiments, modified oligonucleotides are gapmers. In certain embodiments, the gapmers are MOE gapmers. In certain embodiments, all of the internucleoside linkages of the modified oligonucleotides are phosphorothioate internucleoside linkages. In certain embodiments, the internucleoside linkages of the modified oligonucleotides are phosphorothioate internucleoside linkages and phosphodiester internucleoside linkages. In certain embodiments, the phosphodiester (“o”) and phosphorothioate (“s”) internucleoside linkages are arranged in the order from 5′ to 3′: soooossssssssssooss, wherein each “s” represents a phosphorothioate internucleoside linkage and each “o” represents a phosphodiester internucleoside linkage.

The nucleobase sequences of SEQ ID NOs: 73, 136, 197, and 421 are complementary to a portion within nucleobases 5205-5232 of SEQ ID NO: 1.

The nucleobase sequence of Compound Nos.: 1447304, 1447369, 1447481, and 1447520 are complementary to a portion within nucleobases 5205-5232 of SEQ ID NO: 1.

In certain embodiments, modified oligonucleotides complementary to a portion within nucleobases 5205-5232 of SEQ ID NO: 1 achieve at least 75% reduction of CHMP7 RNA in the standard in vitro assay. In certain embodiments, modified oligonucleotides complementary to a portion within nucleobases 5205-5232 of SEQ ID NO: 1 achieve an average of 83% reduction of CHMP7 RNA in the standard in vitro assay.

6. Nucleobases 5404-5430 of SEQ ID NO: 1

In certain embodiments, nucleobases 5404-5430 of SEQ ID NO: 1 comprise a hotspot region. In certain embodiments, modified oligonucleotides are complementary to a portion within nucleobases 5404-5430 of SEQ ID NO: 1. In certain embodiments, modified oligonucleotides are 20 nucleobases in length. In certain embodiments, modified oligonucleotides are gapmers. In certain embodiments, the gapmers are MOE gapmers. In certain embodiments, all of the internucleoside linkages of the modified oligonucleotides are phosphorothioate internucleoside linkages. In certain embodiments, the internucleoside linkages of the modified oligonucleotides are phosphorothioate internucleoside linkages and phosphodiester internucleoside linkages. In certain embodiments, the phosphodiester (“o”) and phosphorothioate (“s”) internucleoside linkages are arranged in the order from 5′ to 3′: soooossssssssssooss, wherein each “s” represents a phosphorothioate internucleoside linkage and each “o” represents a phosphodiester internucleoside linkage.

The nucleobase sequences of SEQ ID NOs: 79, 160, 168, 230, 313, 331, and 464 are complementary to a portion within nucleobases 5404-5430 of SEQ ID NO: 1.

The nucleobase sequence of Compound Nos.: 1447206, 1447236, 1447553, 1447564, 1447595, 1447604, and 1447624 are complementary to a portion within nucleobases 5404-5430 of SEQ ID NO: 1.

In certain embodiments, modified oligonucleotides complementary to a portion within nucleobases 5404-5430 of SEQ ID NO: 1 achieve at least 68% reduction of CHMP7 RNA in the standard in vitro assay. In certain embodiments, modified oligonucleotides complementary to a portion within nucleobases 5404-5430 of SEQ ID NO: 1 achieve an average of 84.3% reduction of CHMP7 RNA in the standard in vitro assay.

7. Nucleobases 8323-8344 of SEQ ID NO: 1

In certain embodiments, nucleobases 8323-8344 of SEQ ID NO: 1 comprise a hotspot region. In certain embodiments, modified oligonucleotides are complementary to a portion within nucleobases 8323-8344 of SEQ ID NO: 1. In certain embodiments, modified oligonucleotides are 20 nucleobases in length. In certain embodiments, modified oligonucleotides are gapmers. In certain embodiments, the gapmers are MOE gapmers. In certain embodiments, all of the internucleoside linkages of the modified oligonucleotides are phosphorothioate internucleoside linkages. In certain embodiments, the internucleoside linkages of the modified oligonucleotides are phosphorothioate internucleoside linkages and phosphodiester internucleoside linkages. In certain embodiments, the phosphodiester (“o”) and phosphorothioate (“s”) internucleoside linkages are arranged in the order from 5′ to 3′: soooossssssssssooss, wherein each “s” represents a phosphorothioate internucleoside linkage and each “o” represents a phosphodiester internucleoside linkage.

The nucleobase sequences of SEQ ID NOs: 157, 186, and 265 are complementary to a portion within nucleobases 8323-8344 of SEQ ID NO: 1.

The nucleobase sequence of Compound Nos.: 1447315, 1447331, and 1447602 are complementary to a portion within nucleobases 8323-8344 of SEQ ID NO: 1.

In certain embodiments, modified oligonucleotides complementary to a portion within nucleobases 8323-8344 of SEQ ID NO: 1 achieve at least 89% reduction of CHMP7 RNA in the standard in vitro assay. In certain embodiments, modified oligonucleotides complementary to a portion within nucleobases 8323-8344 of SEQ ID NO: 1 achieve an average of 92% reduction of CHMP7 RNA in the standard in vitro assay.

8. Nucleobases 16927-16950 of SEQ ID NO: 1

In certain embodiments, nucleobases 16927-16950 of SEQ ID NO: 1 comprise a hotspot region. In certain embodiments, modified oligonucleotides are complementary to a portion within nucleobases 16927-16950 of SEQ ID NO: 1. In certain embodiments, modified oligonucleotides are 20 nucleobases in length. In certain embodiments, modified oligonucleotides are gapmers. In certain embodiments, the gapmers are MOE gapmers. In certain embodiments, all of the internucleoside linkages of the modified oligonucleotides are phosphorothioate internucleoside linkages. In certain embodiments, the internucleoside linkages of the modified oligonucleotides are phosphorothioate internucleoside linkages and phosphodiester internucleoside linkages. In certain embodiments, the phosphodiester (“o”) and phosphorothioate (“s”) internucleoside linkages are arranged in the order from 5′ to 3′: soooossssssssssooss, wherein each “s” represents a phosphorothioate internucleoside linkage and each “o” represents a phosphodiester internucleoside linkage.

The nucleobase sequences of SEQ ID NOs: 128, 182, and 309 are complementary to a portion within nucleobases 16927-16950 of SEQ ID NO: 1.

The nucleobase sequence of Compound Nos.: 1447285, 1447434, and 1447579 are complementary to a portion within nucleobases 16927-16950 of SEQ ID NO: 1.

In certain embodiments, modified oligonucleotides complementary to a portion within nucleobases 16927-16950 of SEQ ID NO: 1 achieve at least 64% reduction of CHMP7 RNA in the standard in vitro assay. In certain embodiments, modified oligonucleotides complementary to a portion within nucleobases 16927-16950 of SEQ ID NO: 1 achieve an average of 70.7% reduction of CHMP7 RNA in the standard in vitro assay.

9. Nucleobases 17298-17340 of SEQ ID NO: 1

In certain embodiments, nucleobases 17298-17340 of SEQ ID NO: 1 comprise a hotspot region. In certain embodiments, modified oligonucleotides are complementary to a portion within nucleobases 17298-17340 of SEQ ID NO: 1. In certain embodiments, modified oligonucleotides are 20 nucleobases in length. In certain embodiments, modified oligonucleotides are gapmers. In certain embodiments, the gapmers are MOE gapmers. In certain embodiments, all of the internucleoside linkages of the modified oligonucleotides are phosphorothioate internucleoside linkages. In certain embodiments, the internucleoside linkages of the modified oligonucleotides are phosphorothioate internucleoside linkages and phosphodiester internucleoside linkages. In certain embodiments, the phosphodiester (“o”) and phosphorothioate (“s”) internucleoside linkages are arranged in the order from 5′ to 3′: soooossssssssssooss, wherein each “s” represents a phosphorothioate internucleoside linkage and each “o” represents a phosphodiester internucleoside linkage.

The nucleobase sequences of SEQ ID NOs: 44, 76, 153, 206, 283, 363, and 416 are complementary to a portion within nucleobases 17298-17340 of SEQ ID NO: 1.

The nucleobase sequence of Compound Nos.: 1447397, 1447283, 1447435, 1447433, 1447416, 1447587, and 1447525 are complementary to a portion within nucleobases 17298-17340 of SEQ ID NO: 1.

In certain embodiments, modified oligonucleotides complementary to a portion within nucleobases 17298-17340 of SEQ ID NO: 1 achieve at least 43% reduction of CHMP7 RNA in the standard in vitro assay. In certain embodiments, modified oligonucleotides complementary to a portion within nucleobases 17298-17340 of SEQ ID NO: 1 achieve an average of 65% reduction of CHMP7 RNA in the standard in vitro assay.

10. Nucleobases 18287-18313 of SEQ ID NO: 1

In certain embodiments, nucleobases 18287-18313 of SEQ ID NO: 1 comprise a hotspot region. In certain embodiments, modified oligonucleotides are complementary to a portion within nucleobases 18287-18313 of SEQ ID NO: 1. In certain embodiments, modified oligonucleotides are 20 nucleobases in length. In certain embodiments, modified oligonucleotides are gapmers. In certain embodiments, the gapmers are MOE gapmers. In certain embodiments, all of the internucleoside linkages of the modified oligonucleotides are phosphorothioate internucleoside linkages. In certain embodiments, the internucleoside linkages of the modified oligonucleotides are phosphorothioate internucleoside linkages and phosphodiester internucleoside linkages. In certain embodiments, the phosphodiester (“o”) and phosphorothioate (“s”) internucleoside linkages are arranged in the order from 5′ to 3′: soooossssssssssooss, wherein each “s” represents a phosphorothioate internucleoside linkage and each “o” represents a phosphodiester internucleoside linkage.

The nucleobase sequences of SEQ ID NOs: 85, 121, 189, 300, and 354 are complementary to a portion within nucleobases 18287-18313 of SEQ ID NO: 1.

The nucleobase sequence of Compound Nos.: 1447326, 1447379, 1447395, 1447535, and 1447599 are complementary to a portion within nucleobases 18287-18313 of SEQ ID NO: 1.

In certain embodiments, modified oligonucleotides complementary to a portion within nucleobases 18287-18313 of SEQ ID NO: 1 achieve at least 63% reduction of CHMP7 RNA in the standard in vitro assay. In certain embodiments, modified oligonucleotides complementary to a portion within nucleobases 18287-18313 of SEQ ID NO: 1 achieve an average of 79% reduction of CHMP7 RNA in the standard in vitro assay.

| | Start | Stop | Min | Max | | | |
|---|---|---|---|---|---|---|---|
| | Site | Site | % | % | Avg % | | |
| | SEQ | SEQ | Red. | Red. | Red. | | |
| Hotspot | ID NO: | ID NO: | In | In | In | Compound Nos. in | SEQ ID NOs in |
| ID | 1 | 1 | vitro | vitro | vitro | range | range |
| 1 | 3950 | 3983 | 84 | 92 | 89 | 1447312, 1447549, | 220, 302, 345 |
| | | | | | | 1447488 | |
| 2 | 4242 | 4266 | 60 | 83 | 68 | 1447338, 1447449, | 21, 131, 191, |
| | | | | | | 1447242, 1447606 | 465 |
| 3 | 4480 | 4525 | 41 | 80 | 61 | 1447297, 1447361, | 34, 116, 184, |
| | | | | | | 1447311, 1447634, | 242, 257, 340, |
| | | | | | | 1447299, 1447279, | 474 |
| | | | | | | 1447636 | |
| 4 | 4534 | 4566 | 58 | 76 | 66 | 1447461, 1447313, | 55, 118, 202, |
| | | | | | | 1447507, 1447343, | 267, 372, 422 |
| | | | | | | 1447400, 1447387 | |
| 5 | 5205 | 5232 | 75 | 90 | 83 | 1447369, 1447481, | 73, 136, 197, |
| | | | | | | 1447520, 1447304 | 421 |
| 6 | 5404 | 5430 | 68 | 99 | 84 | 1447206, 1447624, | 79, 160, 168, |
| | | | | | | 1447553, 1447604, | 230, 313, 331, |
| | | | | | | 1447236, 1447595, | 464 |
| | | | | | | 1447564 | |
| 7 | 8323 | 8344 | 89 | 94 | 92 | 1447315, 1447331, | 157, 186, 265 |
| | | | | | | 1447602 | |
| 8 | 16927 | 16950 | 64 | 77 | 70.7 | 1447285, 1447434, | 128, 182, 309 |
| | | | | | | 1447579 | |
| 9 | 17298 | 17340 | 43 | 82 | 65 | 1447397, 1447283, | 44, 76, 153, 206, |
| | | | | | | 1447435, 1447433, | 283, 363, 416 |
| | | | | | | 1447416, 1447587, | |
| | | | | | | 1447525 | |
| 10 | 18287 | 18313 | 63 | 92 | 79 | 1447379, 1447535, | 85, 121, 189, |
| | | | | | | 1447326, 1447395, | 300, 354 |
| | | | | | | 1447599 | |

### Nonlimiting Disclosure and Incorporation by Reference

Each of the literature and patent publications listed herein is incorporated by reference in its entirety. While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar moiety (2′-OH in place of one 2′-H of DNA) or as an RNA having a modified base (thymine (methylated uracil) in place of a uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as α or β such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms, unless specified otherwise. Likewise, all cis- and trans-isomers and tautomeric forms of the compounds herein are also included unless otherwise indicated. Oligomeric compounds described herein include chirally pure or enriched mixtures as well as racemic mixtures. For example, oligomeric compounds having a plurality of phosphorothioate internucleoside linkages include such compounds in which chirality of the phosphorothioate internucleoside linkages is controlled or is random. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

The following examples illustrate certain embodiments of the present disclosure and are not limiting. Moreover, where specific embodiments are provided, the inventors have contemplated generic application of those specific embodiments.

### Example 1: Effect of 5-10-5 MOE Mixed Backbone Modified Oligonucleotides on Human CHMP7 RNA In Vitro, Single Dose

Modified oligonucleotides complementary to human CHMP7 nucleic acid were designed and tested for their single dose effects on CHMP7 RNA in vitro. The modified oligonucleotides were tested in a series of experiments that had the same culture conditions.

The modified oligonucleotides in the tables below are 5-10-5 MOE gapmers with mixed PO/PS internucleoside linkages. The gapmers are 20 nucleosides in length, wherein the central gap segment consists of ten 2′-β-D-deoxynucleosides, and wherein the 5′ and 3′ wing segments each consist of five 2′-MOE modified nucleosides. The sugar motif for the gapmers is (from 5′ to 3′): eeeeeddddddddddeeeee; wherein ‘d’ represents a 2′-β-D-deoxyribosyl sugar, and ‘e’ represents a 2′-MOE modified sugar moiety. The internucleoside linkage motif for the gapmers is (from to 3′): soooossssssssssooss; wherein each ‘o’ represents a phosphodiester internucleoside linkage and each ‘s’ represents a phosphorothioate internucleoside linkage. Each cytosine residue is a 5-methyl cytosine.

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the tables below is 100% complementary to SEQ ID NO: 1 (ENSEMBLGene ID ENSG00000147457.14 from ENSEMBL Release 101: August 2020), to SEQ ID NO: 2 (GENBANK Accession No. NM_152272.5), or to both. ‘N/A’ indicates that the modified oligonucleotide is not 100% complementary to that particular target nucleic acid sequence.

Cultured A431 cells were treated with modified oligonucleotide at a concentration of 4,000 nM by free uptake at a density of 10,000 cells per well. After a treatment period of approximately 48 hours, total RNA was isolated from the cells and CHMP7 RNA levels were measured by quantitative real-time RTPCR. CHMP7 RNA levels were measured by human primer probe set RTS50844 (forward sequence CAAGTGGACTCTTTCTAACATGC, designated herein as SEQ ID NO: 5; reverse sequence GCGAGTTCTGATACAGACGAT, designated herein as SEQ ID NO: 6; probe sequence CCTCCTCAGCCTTTTCCTTCAACAGC, designated herein as SEQ ID NO: 7). CHMP7 RNA levels were normalized to total RNA content, as measured by RIBOGREEN®. Reduction of CHMP7 RNA is presented in the tables below as percent CHMP7 RNA relative to the amount in untreated control cells (% UTC). Each table represents results from an individual assay plate. The values marked with an “T” indicate that the modified oligonucleotide is complementary to the amplicon region of the primer probe set. Additional assays may be used to measure the potency and efficacy of the modified oligonucleotides complementary to the amplicon region.

TABLE 1
Reduction of CHMP7 RNA by 5-10-5 MOE gapmers with mixed PO/PS internucleoside linkages in A431 cells

| | SEQ ID | SEQ | SEQ ID | SEQ | | | |
|---|---|---|---|---|---|---|---|
| | No: 1 | ID No: | No: 2 | ID No: | | CHMP7 | |
| Compound | Start | 1 Stop | Start | 2 Stop | | (% | SEQ ID |
| Number | Site | Site | Site | Site | Sequence (5′ to 3′) | UTC) | NO |
| 1447185 | 1125 | 1144 | N/A | N/A | GGCATTTTATATTCATGGAC | 88 | 10 |
| 1447189 | 16656 | 16675 | 1917 | 1936 | AGGACAGTTTCTCAAGTTCA | 76 | 11 |
| 1447190 | 10035 | 10054 | N/A | N/A | ATGTGATGCTATTAATAGGA | 24 | 12 |
| 1447191 | 8219 | 8238 | N/A | N/A | AGTACATAGATCTCTGCACA | 49 | 13 |
| 1447202 | 15172 | 15191 | 1685 | 1704 | TTGGATCTGATCCACGAGGC | 88 | 14 |
| 1447203 | 4818 | 4837 | N/A | N/A | AAAGCTGTTAACTATTAGGT | 52 | 15 |
| 1447204 | 56 | 75 | 56 | 75 | GACGAGTATACTCAAAGTCA | 112 | 16 |
| 1447214 | 18276 | 18295 | 3323 | 3342 | CCCCCAACATTTCCGTTTAC | 123 | 17 |
| 1447223 | 1306 | 1325 | N/A | N/A | GTCAATTCCCATATACCTAT | 108 | 18 |
| 1447230 | 6665 | 6684 | N/A | N/A | AAGGACACTACACTCTGAGC | 122 | 19 |
| 1447239 | 10330 | 10349 | N/A | N/A | ATGATGATCCTTTATTCTGT | 38 | 20 |
| 1447242 | 4246 | 4265 | N/A | N/A | ATCAGAGTTGACTTTCTCCT | 40 | 21 |
| 1447244 | 16319 | 16338 | N/A | N/A | TCTTCCACACTCCAAGCTAA | 94 | 22 |
| 1447245 | 16988 | 17007 | 2035 | 2054 | AAGCCTGGTCTCTCTTTTAC | 91 | 23 |
| 1447247 | 17119 | 17138 | 2166 | 2185 | GCATCGCTTCCAGAAATTCT | 66 | 24 |
| 1447262 | 12519 | 12538 | N/A | N/A | TGGTCCAGAACTTTTACCTT | 126 | 25 |
| 1447264 | 3703 | 3722 | N/A | N/A | CTCTTAGACCTTCTGCTCCA | 46 | 26 |
| 1447266 | 5854 | 5873 | N/A | N/A | AACTCAGTTATCAACTCAGT | 89 | 27 |
| 1447276 | 5605 | 5624 | 979 | 998 | CTGCTGTCTACACTGGCCAT | 82 | 28 |
| 1447277 | 17074 | 17093 | 2121 | 2140 | TCCAGATAAAATCAGTGGTT | 138 | 29 |
| 1447288 | 1533 | 1552 | N/A | N/A | TGCTCACACCATTCCTAGTC | 110 | 30 |
| 1447289 | 11791 | 11810 | N/A | N/A | ATGAGCCTCCATACCTTCTC | 84 | 31 |
| 1447296 | 8108 | 8127 | N/A | N/A | TATCGATAGATTATCATGCA | 57 | 32 |
| 1447310 | 18096 | 18115 | 3143 | 3162 | TCGCTTCTTCTCGCCATTGC | 67 | 33 |
| 1447311 | 4485 | 4504 | N/A | N/A | ACTGTATGCCATCTCAGAAA | 53 | 34 |
| 1447314 | 5451 | 5470 | N/A | N/A | TCACGACCATTTGTTAAGCA | 11 | 35 |
| 1447336 | 1695 | 1714 | N/A | N/A | TCCAACACCAGCTCATGATA | 173 | 36 |
| 1447340 | 17545 | 17564 | 2592 | 2611 | CGACCTCTTTCTCTGAACAC | 60 | 37 |
| 1447363 | 4602 | 4621 | N/A | N/A | GGAAGATGATCCACTTCCAA | 137 | 38 |
| 1447368 | 16311 | 16330 | N/A | N/A | ACTCCAAGCTAAGTCACCAA | 78 | 39 |
| 1447371 | 3900 | 3919 | N/A | N/A | ACAGACCTTTTAAGGGCACT | 25 | 40 |
| 1447381 | 16733 | 16752 | N/A | N/A | GCCCATCAACTCTGTCAGCC | 99 | 41 |
| 1447383 | 10652 | 10671 | N/A | N/A | CCATTTCGCTCATACATGGA | 151 | 42 |
| 1447392 | 16951 | 16970 | 1998 | 2017 | GAGGGTCCTACAATGGCTTT | 100 | 43 |
| 1447397 | 17298 | 17317 | 2345 | 2364 | CTGCAAATCTTTCCCCTTCA | 29 | 44 |
| 1447405 | 9530 | 9549 | N/A | N/A | AAGTCCTCAGCATATCCACA | 47 | 45 |
| 1447411 | 7531 | 7550 | N/A | N/A | TGTTTGCCAGTACAAACCTC | 117 | 46 |
| 1447413 | 16415 | 16434 | N/A | N/A | ACCAAAACCCGCTTAGCTTT | 93 | 47 |
| 1447424 | 683 | 702 | N/A | N/A | GTACAGATCTTACACCATTC | 118 | 48 |
| 1447430 | 9034 | 9053 | N/A | N/A | CCTGAAATTCAATAGCCATA | 70 | 49 |
| 1447436 | 5732 | 5751 | N/A | N/A | ACCCACCTTCAACAGCTCCA | 71† | 50 |
| 1447439 | 9291 | 9310 | N/A | N/A | CATCGAGTTTTCATTGATTT | 110 | 51 |
| 1447441 | 13019 | 13038 | N/A | N/A | AAGCCCACATATACTAACAT | 130 | 52 |
| 1447450 | 5814 | 5833 | N/A | N/A | GGTCTTTTTTCAAGAACGCA | 52 | 53 |
| 1447458 | 14167 | 14186 | N/A | N/A | AGGATAAGCTTTTTCAACCC | 74 | 54 |
| 1447461 | 4534 | 4553 | N/A | N/A | CTACTGTTTCTCCTGTTCCC | 41 | 55 |
| 1447463 | 16526 | 16545 | 1787 | 1806 | GATGTCCAATTCCTTCTCCA | 51 | 56 |
| 1447466 | 8001 | 8020 | N/A | N/A | GGTGCACACCAATTCCATTT | 93 | 57 |
| 1447475 | 15482 | 15501 | N/A | N/A | TGTCCACTCACCTAAGCCAT | 160 | 58 |
| 1447476 | 5109 | 5128 | N/A | N/A | CTTCTTGCTCTTCCAAGGCA | 74 | 59 |
| 1447478 | 2094 | 2113 | N/A | N/A | CCAGATGGTTTCATTTGATA | 140 | 60 |
| 1447485 | 4052 | 4071 | N/A | N/A | GAGGATAATTCTATCACCTC | 95 | 61 |
| 1447490 | 18205 | 18224 | 3252 | 3271 | TGGAGCAAATTCTTCTCCTC | 87 | 62 |
| 1447493 | 14124 | 14143 | N/A | N/A | GGCACATTATACACATCTTC | 57 | 63 |
| 1447495 | 14967 | 14986 | N/A | N/A | GCTCAATTTACCCTATACCC | 58 | 64 |
| 1447497 | 11435 | 11454 | N/A | N/A | GATCTGTGTTTTTAAGCCTT | 11 | 65 |
| 1447502 | 5691 | 5710 | 1065 | 1084 | CTGGAACCTTATTATCTCCC | 15† | 66 |
| 1447509 | 16327 | 16346 | N/A | N/A | GAGATGATTCTTCCACACTC | 44 | 67 |
| 1447510 | 11845 | 11864 | N/A | N/A | GGGCTCACTACTCTATGTAC | 163 | 68 |
| 1447512 | 12548 | 12567 | N/A | N/A | TCAGCAAATGATATTTGGTC | 66 | 69 |
| 1447514 | 11830 | 11849 | N/A | N/A | TGTACTTAATCAATCACTGC | 91 | 70 |
| 1447516 | 2031 | 2050 | N/A | N/A | CCATCTCTTTTATAAGATAG | 117 | 71 |
| 1447517 | 9241 | 9260 | N/A | N/A | GTAACTGCACAGTAACCACA | 59 | 72 |
| 1447520 | 5212 | 5231 | N/A | N/A | GGTGAGATCTCCTTTTACAA | 25 | 73 |
| 1447523 | 14029 | 14048 | N/A | N/A | CTAGACTTTCCCACCTGGAA | 58 | 74 |
| 1447524 | 18068 | 18087 | 3115 | 3134 | TCAGACCCTTTCCCGTCTGT | 80 | 75 |
| 1447525 | 17321 | 17340 | 2368 | 2387 | GAACTGATTCAGATTTGGCA | 35 | 76 |
| 1447540 | 11248 | 11267 | N/A | N/A | CTGAGGCCTGATTTCCAGCC | 105 | 77 |
| 1447544 | 16269 | 16288 | N/A | N/A | ATGGACCTGACCCTGATTCC | 62 | 78 |
| 1447553 | 5406 | 5425 | N/A | N/A | CATTGTTTATACTCCAGCTC | 32 | 79 |
| 1447570 | 4548 | 4567 | N/A | N/A | ACTGACTTCTTCAACTACTG | 65 | 80 |
| 1447571 | 12898 | 12917 | 1380 | 1399 | GAAGCTGTTCACTCTGCATC | 77 | 81 |
| 1447572 | 748 | 767 | N/A | N/A | AGTCTTTTCAACAATGAGCA | 81 | 82 |
| 1447581 | 11152 | 11171 | N/A | N/A | CATCTAATTCCCTTCCAATT | 70 | 83 |
| 1447598 | 11195 | 11214 | N/A | N/A | ACTAGTCCTCAGTATCACTC | 59 | 84 |
| 1447599 | 18294 | 18313 | 3341 | 3360 | AGTGATTGTTTCTCTTCACC | 37 | 85 |
| 1447623 | 925 | 944 | N/A | N/A | ACGGATACAACCCAACTTCA | 75 | 86 |
| 1447632 | 12820 | 12839 | N/A | N/A | GGGCAAACTTCACAATCTGA | 88 | 87 |

TABLE 2
Reduction of CHMP7 RNA by 5-10-5 MOE gapmers with mixed PO/PS internucleoside linkages in A431 cells

| | SEQ ID | SEQ | SEQ ID | SEQ | | | |
|---|---|---|---|---|---|---|---|
| | No: 1 | ID No: | No: 2 | ID No: | | CHMP7 | |
| Compound | Start | 1 Stop | Start | 2 Stop | | (% | SEQ ID |
| Number | Site | Site | Site | Site | Sequence (5′ to 3′) | UTC) | NO |
| 1447199 | 11193 | 11212 | N/A | N/A | TAGTCCTCAGTATCACTCTC | 53 | 88 |
| 1447201 | 1299 | 1318 | N/A | N/A | CCCATATACCTATAATGATC | 136 | 89 |
| 1447209 | 52 | 71 | 52 | 71 | AGTATACTCAAAGTCAGGCA | 82 | 90 |
| 1447211 | 16250 | 16269 | N/A | N/A | CCACTGCCCTTGAAGGAGGA | 121 | 91 |
| 1447229 | 14166 | 14185 | N/A | N/A | GGATAAGCTTTTTCAACCCA | 43 | 92 |
| 1447240 | 12897 | 12916 | 1379 | 1398 | AAGCTGTTCACTCTGCATCA | 119 | 93 |
| 1447241 | 17116 | 17135 | 2163 | 2182 | TCGCTTCCAGAAATTCTATC | 49 | 94 |
| 1447243 | 16287 | 16306 | N/A | N/A | GTGTATTCTGTTAACACCAT | 63 | 95 |
| 1447252 | 11338 | 11357 | N/A | N/A | GGGATTTTACACTCCTGCCT | 44 | 96 |
| 1447257 | 5813 | 5832 | N/A | N/A | GTCTTTTTTCAAGAACGCAC | 30 | 97 |
| 1447259 | 4051 | 4070 | N/A | N/A | AGGATAATTCTATCACCTCA | 35 | 98 |
| 1447260 | 5724 | 5743 | 1098 | 1117 | TCAACAGCTCCACAGCGACA | 25† | 99 |
| 1447261 | 670 | 689 | N/A | N/A | ACCATTCACAAAAGCCAGCT | 155 | 100 |
| 1447267 | 10329 | 10348 | N/A | N/A | TGATGATCCTTTATTCTGTA | 34 | 101 |
| 1447271 | 9612 | 9631 | N/A | N/A | TCCAAGATTATGTATACAAC | 41 | 102 |
| 1447306 | 11844 | 11863 | N/A | N/A | GGCTCACTACTCTATGTACT | 68 | 103 |
| 1447309 | 3617 | 3636 | N/A | N/A | ACTGTCACCTCTGTTAGGCT | 15 | 104 |
| 1447317 | 16317 | 16336 | N/A | N/A | TTCCACACTCCAAGCTAAGT | 87 | 105 |
| 1447319 | 16653 | 16672 | 1914 | 1933 | ACAGTTTCTCAAGTTCAGCT | 46 | 106 |
| 1447322 | 2093 | 2112 | N/A | N/A | CAGATGGTTTCATTTGATAA | 143 | 107 |
| 1447325 | 7528 | 7547 | N/A | N/A | TTGCCAGTACAAACCTCATC | 38 | 108 |
| 1447327 | 810 | 829 | N/A | N/A | GGCACATGTAAATACAGTCA | 157 | 109 |
| 1447329 | 15470 | 15489 | 1748 | 1767 | TAAGCCATTTGTTACCCCAC | 41 | 110 |
| 1447334 | 1993 | 2012 | N/A | N/A | CCAACTGATCCTATGAGGCA | 90 | 111 |
| 1447335 | 16402 | 16421 | N/A | N/A | TAGCTTTACAAAAGATGCCA | 89 | 112 |
| 1447349 | 747 | 766 | N/A | N/A | GTCTTTTCAACAATGAGCAC | 61 | 113 |
| 1447351 | 9521 | 9540 | N/A | N/A | GCATATCCACAGCAAAGATC | 47 | 114 |
| 1447360 | 17930 | 17949 | 2977 | 2996 | CCTGCTTCTATTGCACATCC | 74 | 115 |
| 1447361 | 4482 | 4501 | N/A | N/A | GTATGCCATCTCAGAAAGCC | 29 | 116 |
| 1447376 | 12518 | 12537 | N/A | N/A | GGTCCAGAACTTTTACCTTT | 87 | 117 |
| 1447387 | 4547 | 4566 | N/A | N/A | CTGACTTCTTCAACTACTGT | 38 | 118 |
| 1447390 | 17543 | 17562 | 2590 | 2609 | ACCTCTTTCTCTGAACACCT | 42 | 119 |
| 1447394 | 11242 | 11261 | N/A | N/A | CCTGATTTCCAGCCTAGCCT | 70 | 120 |
| 1447395 | 18292 | 18311 | 3339 | 3358 | TGATTGTTTCTCTTCACCCC | 25 | 121 |
| 1447404 | 4599 | 4618 | N/A | N/A | AGATGATCCACTTCCAATAC | 90 | 122 |
| 1447407 | 18078 | 18097 | 3125 | 3144 | GCCAGAGCATTCAGACCCTT | 44 | 123 |
| 1447412 | 8000 | 8019 | N/A | N/A | GTGCACACCAATTCCATTTC | 128 | 124 |
| 1447423 | 12814 | 12833 | N/A | N/A | ACTTCACAATCTGAGGGACA | 130 | 125 |
| 1447431 | 4774 | 4793 | N/A | N/A | TGACACTGCTTTTAATACTA | 7 | 126 |
| 1447432 | 18204 | 18223 | 3251 | 3270 | GGAGCAAATTCTTCTCCTCT | 70 | 127 |
| 1447434 | 16931 | 16950 | 1978 | 1997 | AGAGTCGGTTCCAATTGCCT | 29 | 128 |
| 1447444 | 11729 | 11748 | 1238 | 1257 | CTGCAGCAACACCAAGTAGA | 116 | 129 |
| 1447448 | 4533 | 4552 | N/A | N/A | TACTGTTTCTCCTGTTCCCT | 51 | 130 |
| 1447449 | 4243 | 4262 | N/A | N/A | AGAGTTGACTTTCTCCTTCC | 17 | 131 |
| 1447460 | 17045 | 17064 | 2092 | 2111 | CCTCTTCCAAACACATTCTG | 96 | 132 |
| 1447462 | 11113 | 11132 | N/A | N/A | GCCAATTGGCTATACTGCAA | 71 | 133 |
| 1447471 | 8105 | 8124 | N/A | N/A | CGATAGATTATCATGCAGGC | 9 | 134 |
| 1447472 | 1694 | 1713 | N/A | N/A | CCAACACCAGCTCATGATAA | 62 | 135 |
| 1447481 | 5207 | 5226 | N/A | N/A | GATCTCCTTTTACAATTGGT | 10 | 136 |
| 1447491 | 9236 | 9255 | N/A | N/A | TGCACAGTAACCACACACAA | 89 | 137 |
| 1447500 | 16984 | 17003 | 2031 | 2050 | CTGGTCTCTCTTTTACATGA | 48 | 138 |
| 1447501 | 5853 | 5872 | N/A | N/A | ACTCAGTTATCAACTCAGTA | 112 | 139 |
| 1447503 | 18274 | 18293 | 3321 | 3340 | CCCAACATTTCCGTTTACCA | 66 | 140 |
| 1447511 | 5588 | 5607 | 962 | 981 | CATGAAGTCTGACTCCCGCT | 132 | 141 |
| 1447518 | 16524 | 16543 | 1785 | 1804 | TGTCCAATTCCTTCTCCAGT | 83 | 142 |
| 1447522 | 5108 | 5127 | N/A | N/A | TTCTTGCTCTTCCAAGGCAA | 91 | 143 |
| 1447526 | 16708 | 16727 | N/A | N/A | GCCATATGCCCTCCAAAGGC | 133 | 144 |
| 1447527 | 11829 | 11848 | N/A | N/A | GTACTTAATCAATCACTGCT | 71 | 145 |
| 1447528 | 14123 | 14142 | N/A | N/A | GCACATTATACACATCTTCC | 35 | 146 |
| 1447531 | 1509 | 1528 | N/A | N/A | CTGTACGGGAAATCCTAGCT | 100 | 147 |
| 1447532 | 12547 | 12566 | N/A | N/A | CAGCAAATGATATTTGGTCT | 23 | 148 |
| 1447537 | 1058 | 1077 | N/A | N/A | GTAGATCCAGTGAAATCCCA | 59 | 149 |
| 1447542 | 5425 | 5444 | N/A | N/A | CTGTGCCAACGACACGCAGC | 91 | 150 |
| 1447574 | 13017 | 13036 | N/A | N/A | GCCCACATATACTAACATTT | 102 | 151 |
| 1447575 | 15136 | 15155 | 1649 | 1668 | TGTGACATCCTTCATGGAGA | 82 | 152 |
| 1447587 | 17320 | 17339 | 2367 | 2386 | AACTGATTCAGATTTGGCAA | 47 | 153 |
| 1447593 | 9283 | 9302 | N/A | N/A | TTTCATTGATTTGACTGCCC | 52 | 154 |
| 1447596 | 6664 | 6683 | N/A | N/A | AGGACACTACACTCTGAGCA | 51 | 155 |
| 1447600 | 10592 | 10611 | N/A | N/A | ATGCTATGAAAATATAGGGA | 56 | 156 |
| 1447602 | 8325 | 8344 | N/A | N/A | GTGCAACTTATTACAAACTT | 11 | 157 |
| 1447603 | 14008 | 14027 | N/A | N/A | ACAGACTTCAATGTCTGTGT | 56 | 158 |
| 1447619 | 14855 | 14874 | N/A | N/A | TGCAAGTGACCAACACACAC | 104 | 159 |
| 1447624 | 5405 | 5424 | N/A | N/A | ATTGTTTATACTCCAGCTCT | 18 | 160 |
| 1447626 | 17293 | 17312 | 2340 | 2359 | AATCTTTCCCCTTCATGGGC | 69 | 161 |
| 1447635 | 8218 | 8237 | N/A | N/A | GTACATAGATCTCTGCACAA | 52 | 162 |
| 1447643 | 16326 | 16345 | N/A | N/A | AGATGATTCTTCCACACTCC | 60 | 163 |
| 1447646 | 3781 | 3800 | N/A | N/A | TGGCAAGATGCCTACAGGCC | 81 | 164 |
| 1447647 | 5690 | 5709 | 1064 | 1083 | TGGAACCTTATTATCTCCCA | 27† | 165 |

TABLE 3
Reduction of CHMP7 RNA by 5-10-5 MOE gapmers with mixed PO/PS internucleoside linkages in A431 cells

| | SEQ ID | SEQ | SEQ ID | SEQ | | | |
|---|---|---|---|---|---|---|---|
| | No: 1 | ID No: | No: 2 | ID No: | | CHMP7 | |
| Compound | Start | 1 Stop | Start | 2 Stop | | (% | SEQ ID |
| Number | Site | Site | Site | Site | Sequence (5′ to 3′) | UTC) | NO |
| 1447193 | 2048 | 2067 | N/A | N/A | GTTGTACCAGTTCTGTGCCA | 78 | 166 |
| 1447197 | 10327 | 10346 | N/A | N/A | ATGATCCTTTATTCTGTAGA | 37 | 167 |
| 1447206 | 5404 | 5423 | N/A | N/A | TTGTTTATACTCCAGCTCTA | 29 | 168 |
| 1447208 | 15115 | 15134 | 1628 | 1647 | TTTGAGTGCTCCTACCCCAG | 64 | 169 |
| 1447210 | 11190 | 11209 | N/A | N/A | TCCTCAGTATCACTCTCTGA | 62 | 170 |
| 1447212 | 11205 | 11224 | N/A | N/A | TCACAGCCTCACTAGTCCTC | 86 | 171 |
| 1447216 | 11617 | 11636 | 1126 | 1145 | AGACGATACACCTCCTCAGC | 59† | 172 |
| 1447217 | 16316 | 16335 | N/A | N/A | TCCACACTCCAAGCTAAGTC | 51 | 173 |
| 1447222 | 1822 | 1841 | N/A | N/A | AAGCGGAATTTATTACAGCT | 75 | 174 |
| 1447226 | 16249 | 16268 | N/A | N/A | CACTGCCCTTGAAGGAGGAT | 67 | 175 |
| 1447227 | 13010 | 13029 | N/A | N/A | TATACTAACATTTTGCTGGC | 97 | 176 |
| 1447233 | 9611 | 9630 | N/A | N/A | CCAAGATTATGTATACAACC | 20 | 177 |
| 1447234 | 809 | 828 | N/A | N/A | GCACATGTAAATACAGTCAA | 61 | 178 |
| 1447273 | 13975 | 13994 | N/A | N/A | GGTGTGTCTTTATTAGGGAT | 16 | 179 |
| 1447274 | 9282 | 9301 | N/A | N/A | TTCATTGATTTGACTGCCCT | 52 | 180 |
| 1447280 | 5993 | 6012 | N/A | N/A | GCTGCTTAACATTAATCCCT | 41 | 181 |
| 1447285 | 16930 | 16949 | 1977 | 1996 | GAGTCGGTTCCAATTGCCTT | 23 | 182 |
| 1447292 | 17008 | 17027 | 2055 | 2074 | TAACTATGTACACACCCAGC | 43 | 183 |
| 1447297 | 4480 | 4499 | N/A | N/A | ATGCCATCTCAGAAAGCCTC | 31 | 184 |
| 1447303 | 15469 | 15488 | 1747 | 1766 | AAGCCATTTGTTACCCCACC | 43 | 185 |
| 1447315 | 8324 | 8343 | N/A | N/A | TGCAACTTATTACAAACTTT | 7 | 186 |
| 1447316 | 16624 | 16643 | 1885 | 1904 | GCATCTGAGATCCTAGGGTT | 54 | 187 |
| 1447324 | 16692 | 16711 | N/A | N/A | AGGCCTTGGAAAACAGCTCC | 83 | 188 |
| 1447326 | 18291 | 18310 | 3338 | 3357 | GATTGTTTCTCTTCACCCCC | 8 | 189 |
| 1447337 | 669 | 688 | N/A | N/A | CCATTCACAAAAGCCAGCTC | 93 | 190 |
| 1447338 | 4242 | 4261 | N/A | N/A | GAGTTGACTTTCTCCTTCCC | 37 | 191 |
| 1447341 | 12792 | 12811 | N/A | N/A | GAGAAATTACTACTGCTGCT | 90 | 192 |
| 1447346 | 16385 | 16404 | N/A | N/A | CCAGGCCTCCTTGCAGATTA | 148 | 193 |
| 1447348 | 11337 | 11356 | N/A | N/A | GGATTTTACACTCCTGCCTC | 95 | 194 |
| 1447355 | 16325 | 16344 | N/A | N/A | GATGATTCTTCCACACTCCA | 35 | 195 |
| 1447362 | 4554 | 4573 | N/A | N/A | TCCCTCACTGACTTCTTCAA | 48 | 196 |
| 1447369 | 5205 | 5224 | N/A | N/A | TCTCCTTTTACAATTGGTGC | 19 | 197 |
| 1447372 | 5516 | 5535 | N/A | N/A | ATGCAATGTCCCATTCCAGT | 83 | 198 |
| 1447389 | 16284 | 16303 | N/A | N/A | TATTCTGTTAACACCATGGA | 47 | 199 |
| 1447393 | 12482 | 12501 | N/A | N/A | CTTGACCGCTTCTCTTTGGT | 81 | 200 |
| 1447396 | 1651 | 1670 | N/A | N/A | GAGGGACACATCCTCCACTT | 75 | 201 |
| 1447400 | 4546 | 4565 | N/A | N/A | TGACTTCTTCAACTACTGTT | 42 | 202 |
| 1447403 | 16980 | 16999 | 2027 | 2046 | TCTCTCTTTTACATGAGGGT | 66 | 203 |
| 1447408 | 1054 | 1073 | N/A | N/A | ATCCAGTGAAATCCCAGTGA | 76 | 204 |
| 1447415 | 8075 | 8094 | N/A | N/A | ATCGAGTAAATCCATACTGT | 46 | 205 |
| 1447416 | 17304 | 17323 | 2351 | 2370 | GCAAAGCTGCAAATCTTTCC | 57 | 206 |
| 1447419 | 4730 | 4749 | N/A | N/A | GAGAAGTATCATCCTCAGAA | 45 | 207 |
| 1447421 | 18269 | 18288 | 3316 | 3335 | CATTTCCGTTTACCAAGGTC | 22 | 208 |
| 1447422 | 688 | 707 | N/A | N/A | TCCAGGTACAGATCTTACAC | 87 | 209 |
| 1447442 | 11795 | 11814 | N/A | N/A | ACAGATGAGCCTCCATACCT | 76 | 210 |
| 1447453 | 7944 | 7963 | N/A | N/A | CGTGTGTATTCACTCACTCA | 94 | 211 |
| 1447455 | 4955 | 4974 | N/A | N/A | ACCTAGGTACAATTATCATC | 63 | 212 |
| 1447459 | 1503 | 1522 | N/A | N/A | GGGAAATCCTAGCTAACACA | 70 | 213 |
| 1447464 | 12535 | 12554 | N/A | N/A | TTTGGTCTTCCTTATTTGGT | 53 | 214 |
| 1447465 | 14084 | 14103 | N/A | N/A | AGGAACTGTTAACACCAACA | 47 | 215 |
| 1447469 | 17494 | 17513 | 2541 | 2560 | AAACCTTTTATACTACATGT | 65 | 216 |
| 1447470 | 7527 | 7546 | N/A | N/A | TGCCAGTACAAACCTCATCT | 32 | 217 |
| 1447482 | 11108 | 11127 | N/A | N/A | TTGGCTATACTGCAAACACA | 47 | 218 |
| 1447487 | 18197 | 18216 | 3244 | 3263 | ATTCTTCTCCTCTGCTCTAC | 64 | 219 |
| 1447488 | 3964 | 3983 | N/A | N/A | GTATTAGTTATCAATGTTAC | 8 | 220 |
| 1447498 | 2212 | 2231 | N/A | N/A | CACATTTCTTGAAAGCACAC | 78 | 221 |
| 1447513 | 11843 | 11862 | N/A | N/A | GCTCACTACTCTATGTACTT | 53 | 222 |
| 1447529 | 5718 | 5737 | 1092 | 1111 | GCTCCACAGCGACAAGGACC | 10† | 223 |
| 1447547 | 9151 | 9170 | N/A | N/A | ATAGAGCTTTCCCCACCACA | 54 | 224 |
| 1447550 | 14850 | 14869 | N/A | N/A | GTGACCAACACACACGGAGA | 57 | 225 |
| 1447554 | 9383 | 9402 | N/A | N/A | CGTCATGTAAACAAATTCAA | 47 | 226 |
| 1447555 | 5688 | 5707 | 1062 | 1081 | GAACCTTATTATCTCCCAGC | 4† | 227 |
| 1447556 | 16511 | 16530 | 1772 | 1791 | CTCCAGTTCTTCACTGTCAA | 60 | 228 |
| 1447560 | 5849 | 5868 | N/A | N/A | AGTTATCAACTCAGTAGCCA | 20 | 229 |
| 1447564 | 5411 | 5430 | N/A | N/A | CGCAGCATTGTTTATACTCC | 26 | 230 |
| 1447576 | 17743 | 17762 | 2790 | 2809 | AACAATTTTGCCTCTTTGCA | 73 | 231 |
| 1447588 | 36 | 55 | 36 | 55 | GGCAAGCAAGTTTATTGACC | 63 | 232 |
| 1447591 | 5812 | 5831 | N/A | N/A | TCTTTTTTCAAGAACGCACA | 70 | 233 |
| 1447592 | 12889 | 12908 | 1371 | 1390 | CACTCTGCATCAGCTGGTAC | 104 | 234 |
| 1447601 | 10343 | 10362 | N/A | N/A | AGTGTAATATCTTATGATGA | 59 | 235 |
| 1447607 | 17292 | 17311 | 2339 | 2358 | ATCTTTCCCCTTCATGGGCT | 59 | 236 |
| 1447608 | 14165 | 14184 | N/A | N/A | GATAAGCTTTTTCAACCCAC | 50 | 237 |
| 1447612 | 18077 | 18096 | 3124 | 3143 | CCAGAGCATTCAGACCCTTT | 48 | 238 |
| 1447614 | 1218 | 1237 | N/A | N/A | GGTATAAATCTAACACGGTA | 83 | 239 |
| 1447616 | 8152 | 8171 | N/A | N/A | TTGAAATGTCCATCTGCGGA | 41 | 240 |
| 1447629 | 3743 | 3762 | N/A | N/A | GCTTTTTAGCTCTAACCTTC | 29 | 241 |
| 1447636 | 4506 | 4525 | N/A | N/A | GCATGAACGAAAACGGTTTC | 48 | 242 |
| 1447638 | 17111 | 17130 | 2158 | 2177 | TCCAGAAATTCTATCTGTCC | 59 | 243 |

TABLE 4
Reduction of CHMP7 RNA by 5-10-5 MOE gapmers with mixed PO/PS internucleoside linkages in A431 cells

| | SEQ ID | SEQ | SEQ ID | SEQ | | | |
|---|---|---|---|---|---|---|---|
| | No: 1 | ID No: | No: 2 | ID No: | | CHMP7 | |
| Compound | Start | 1 Stop | Start | 2 Stop | | (% | SEQ ID |
| Number | Site | Site | Site | Site | Sequence (5′ to 3′) | UTC) | NO |
| 1447188 | 12534 | 12553 | N/A | N/A | TTGGTCTTCCTTATTTGGTC | 35 | 244 |
| 1447205 | 659 | 678 | N/A | N/A | AAGCCAGCTCAAATGGATCA | 97 | 245 |
| 1447213 | 10276 | 10295 | N/A | N/A | GCTAGATTAACAGAAAGCTA | 66 | 246 |
| 1447218 | 5687 | 5706 | 1061 | 1080 | AACCTTATTATCTCCCAGCA | 8† | 247 |
| 1447228 | 4363 | 4382 | N/A | N/A | GCAATATCAATGTCCAGTCC | 14 | 248 |
| 1447231 | 18194 | 18213 | 3241 | 3260 | CTTCTCCTCTGCTCTACGCC | 50 | 249 |
| 1447238 | 9610 | 9629 | N/A | N/A | CAAGATTATGTATACAACCT | 49 | 250 |
| 1447249 | 11335 | 11354 | N/A | N/A | ATTTTACACTCCTGCCTCTC | 64 | 251 |
| 1447251 | 18075 | 18094 | 3122 | 3141 | AGAGCATTCAGACCCTTTCC | 34 | 252 |
| 1447255 | 11794 | 11813 | N/A | N/A | CAGATGAGCCTCCATACCTT | 106 | 253 |
| 1447256 | 5379 | 5398 | N/A | N/A | CACAACACTTCTGCATGGGA | 5 | 254 |
| 1447265 | 1203 | 1222 | N/A | N/A | CGGTATGTACAAATTCTACA | 98 | 255 |
| 1447270 | 17548 | 17567 | 2595 | 2614 | GGGCGACCTCTTTCTCTGAA | 59 | 256 |
| 1447279 | 4498 | 4517 | N/A | N/A | GAAAACGGTTTCCACTGTAT | 20 | 257 |
| 1447281 | 16979 | 16998 | 2026 | 2045 | CTCTCTTTTACATGAGGGTC | 75 | 258 |
| 1447286 | 18232 | 18251 | 3279 | 3298 | ATTGAACAACGATTTGTGCT | 36 | 259 |
| 1447287 | 9281 | 9300 | N/A | N/A | TCATTGATTTGACTGCCCTA | 24 | 260 |
| 1447290 | 17222 | 17241 | 2269 | 2288 | CTGCAGGCTATGAAATGACA | 67 | 261 |
| 1447291 | 5515 | 5534 | N/A | N/A | TGCAATGTCCCATTCCAGTC | 46 | 262 |
| 1447294 | 11839 | 11858 | N/A | N/A | ACTACTCTATGTACTTAATC | 79 | 263 |
| 1447323 | 5777 | 5796 | N/A | N/A | ATGCTGTGATCACACCAGGT | 50 | 264 |
| 1447331 | 8323 | 8342 | N/A | N/A | GCAACTTATTACAAACTTTT | 6 | 265 |
| 1447342 | 14057 | 14076 | N/A | N/A | CGGACATATCCTCTGCCTCC | 93 | 266 |
| 1447343 | 4545 | 4564 | N/A | N/A | GACTTCTTCAACTACTGTTT | 24 | 267 |
| 1447344 | 12481 | 12500 | N/A | N/A | TTGACCGCTTCTCTTTGGTA | 111 | 268 |
| 1447345 | 1494 | 1513 | N/A | N/A | TAGCTAACACAGTTAGCCAA | 113 | 269 |
| 1447356 | 1649 | 1668 | N/A | N/A | GGGACACATCCTCCACTTGA | 112 | 270 |
| 1447359 | 14973 | 14992 | N/A | N/A | ACCCAAGCTCAATTTACCCT | 86 | 271 |
| 1447364 | 10340 | 10359 | N/A | N/A | GTAATATCTTATGATGATCC | 17 | 272 |
| 1447366 | 16568 | 16587 | 1829 | 1848 | GTCAGGCAGATCCAAAGGTT | 63 | 273 |
| 1447370 | 4725 | 4744 | N/A | N/A | GTATCATCCTCAGAAATTCT | 18 | 274 |
| 1447373 | 12597 | 12616 | N/A | N/A | CACAATTTTTCCTATAGAAG | 84 | 275 |
| 1447382 | 12880 | 12899 | 1362 | 1381 | TCAGCTGGTACACCCCAACA | 104 | 276 |
| 1447384 | 10658 | 10677 | N/A | N/A | ATGTCACCATTTCGCTCATA | 29 | 277 |
| 1447388 | 1821 | 1840 | N/A | N/A | AGCGGAATTTATTACAGCTA | 107 | 278 |
| 1447391 | 16510 | 16529 | 1771 | 1790 | TCCAGTTCTTCACTGTCAAA | 69 | 279 |
| 1447410 | 9358 | 9377 | N/A | N/A | TTGGACATTTCAAGAAGTGA | 57 | 280 |
| 1447425 | 5818 | 5837 | N/A | N/A | AGACGGTCTTTTTTCAAGAA | 34 | 281 |
| 1447427 | 16324 | 16343 | N/A | N/A | ATGATTCTTCCACACTCCAA | 41 | 282 |
| 1447433 | 17302 | 17321 | 2349 | 2368 | AAAGCTGCAAATCTTTCCCC | 18 | 283 |
| 1447438 | 8129 | 8148 | N/A | N/A | CCTGTTGCATAAATTGTGGC | 64 | 284 |
| 1447440 | 7500 | 7519 | N/A | N/A | TCCATGGGTTTACCTCTCCT | 45 | 285 |
| 1447445 | 797 | 816 | N/A | N/A | ACAGTCAACTGACCTTTCGA | 68 | 286 |
| 1447446 | 13961 | 13980 | N/A | N/A | AGGGATTCCTTGTACAGTCA | 45 | 287 |
| 1447447 | 35 | 54 | 35 | 54 | GCAAGCAAGTTTATTGACCT | 56 | 288 |
| 1447451 | 15465 | 15484 | 1743 | 1762 | CATTTGTTACCCCACCAGCC | 59 | 289 |
| 1447452 | 1044 | 1063 | N/A | N/A | ATCCCAGTGAAAATCATGGC | 61 | 290 |
| 1447473 | 2036 | 2055 | N/A | N/A | CTGTGCCATCTCTTTTATAA | 107 | 291 |
| 1447479 | 11603 | 11622 | N/A | N/A | CTCAGCCTTTTCCTGTGGGA | 83† | 292 |
| 1447483 | 5990 | 6009 | N/A | N/A | GCTTAACATTAATCCCTACA | 30 | 293 |
| 1447486 | 687 | 706 | N/A | N/A | CCAGGTACAGATCTTACACC | 83 | 294 |
| 1447496 | 8040 | 8059 | N/A | N/A | AGGCACCTGATACAATGCAA | 33 | 295 |
| 1447499 | 4954 | 4973 | N/A | N/A | CCTAGGTACAATTATCATCA | 84 | 296 |
| 1447508 | 11200 | 11219 | N/A | N/A | GCCTCACTAGTCCTCAGTAT | 122 | 297 |
| 1447515 | 13009 | 13028 | N/A | N/A | ATACTAACATTTTGCTGGCT | 76 | 298 |
| 1447533 | 4553 | 4572 | N/A | N/A | CCCTCACTGACTTCTTCAAC | 73 | 299 |
| 1447535 | 18288 | 18307 | 3335 | 3354 | TGTTTCTCTTCACCCCCAAC | 19 | 300 |
| 1447548 | 2198 | 2217 | N/A | N/A | GCACACTCTGCCAAAACACA | 133 | 301 |
| 1447549 | 3961 | 3980 | N/A | N/A | TTAGTTATCAATGTTACCCT | 10 | 302 |
| 1447551 | 3720 | 3739 | N/A | N/A | AGGTTAATGAATCCTATCTC | 58 | 303 |
| 1447562 | 9150 | 9169 | N/A | N/A | TAGAGCTTTCCCCACCACAT | 53 | 304 |
| 1447563 | 4136 | 4155 | N/A | N/A | GTCCCGGTATCACCTTTAAC | 53 | 305 |
| 1447565 | 16315 | 16334 | N/A | N/A | CCACACTCCAAGCTAAGTCA | 50 | 306 |
| 1447567 | 16330 | 16349 | N/A | N/A | GCAGAGATGATTCTTCCACA | 11 | 307 |
| 1447578 | 16068 | 16087 | N/A | N/A | TGTCAAACTCATCACAGCAC | 65 | 308 |
| 1447579 | 16927 | 16946 | 1974 | 1993 | TCGGTTCCAATTGCCTTTTT | 36 | 309 |
| 1447584 | 17493 | 17512 | 2540 | 2559 | AACCTTTTATACTACATGTT | 52 | 310 |
| 1447586 | 7914 | 7933 | N/A | N/A | ATTGACTGTCTACCAGGTAT | 34 | 311 |
| 1447594 | 17007 | 17026 | 2054 | 2073 | AACTATGTACACACCCAGCA | 31 | 312 |
| 1447595 | 5409 | 5428 | N/A | N/A | CAGCATTGTTTATACTCCAG | 1 | 313 |
| 1447610 | 11189 | 11208 | N/A | N/A | CCTCAGTATCACTCTCTGAA | 100 | 314 |
| 1447622 | 14760 | 14779 | 1555 | 1574 | AGGATGCCTTGAACAGTGTC | 86 | 315 |
| 1447630 | 14164 | 14183 | N/A | N/A | ATAAGCTTTTTCAACCCACA | 51 | 316 |
| 1447639 | 5694 | 5713 | 1068 | 1087 | CAGCTGGAACCTTATTATCT | 74† | 317 |
| 1447642 | 16686 | 16705 | N/A | N/A | TGGAAAACAGCTCCATACCT | 76 | 318 |
| 1447645 | 17110 | 17129 | 2157 | 2176 | CCAGAAATTCTATCTGTCCT | 56 | 319 |
| 1447648 | 5115 | 5134 | N/A | N/A | GAAGGACTTCTTGCTCTTCC | 49 | 320 |
| 1447650 | 16283 | 16302 | N/A | N/A | ATTCTGTTAACACCATGGAC | 109 | 321 |

TABLE 5
Reduction of CHMP7 RNA by 5-10-5 MOE gapmers with mixed PO/PS internucleoside linkages in A431 cells

| | SEQ ID | SEQ | SEQ ID | SEQ | | | |
|---|---|---|---|---|---|---|---|
| | No: 1 | ID No: | No: 2 | ID No: | | CHMP7 | |
| Compound | Start | 1 Stop | Start | 2 Stop | | (% | SEQ ID |
| Number | Site | Site | Site | Site | Sequence (5′ to 3′) | UTC) | NO |
| 1447186 | 12533 | 12552 | N/A | N/A | TGGTCTTCCTTATTTGGTCC | 38 | 322 |
| 1447194 | 16529 | 16548 | 1790 | 1809 | GAGGATGTCCAATTCCTTCT | 80 | 323 |
| 1447195 | 11334 | 11353 | N/A | N/A | TTTTACACTCCTGCCTCTCA | 65 | 324 |
| 1447198 | 5514 | 5533 | N/A | N/A | GCAATGTCCCATTCCAGTCT | 46 | 325 |
| 1447207 | 4362 | 4381 | N/A | N/A | CAATATCAATGTCCAGTCCA | 57 | 326 |
| 1447215 | 5735 | 5754 | N/A | N/A | AGTACCCACCTTCAACAGCT | 81† | 327 |
| 1447220 | 12879 | 12898 | 1361 | 1380 | CAGCTGGTACACCCCAACAT | 100 | 328 |
| 1447224 | 5297 | 5316 | N/A | N/A | GCCAAATTTACACCATGATA | 9 | 329 |
| 1447235 | 13008 | 13027 | N/A | N/A | TACTAACATTTTGCTGGCTC | 57 | 330 |
| 1447236 | 5408 | 5427 | N/A | N/A | AGCATTGTTTATACTCCAGC | 2 | 331 |
| 1447246 | 16329 | 16348 | N/A | N/A | CAGAGATGATTCTTCCACAC | 44 | 332 |
| 1447248 | 10656 | 10675 | N/A | N/A | GTCACCATTTCGCTCATACA | 19 | 333 |
| 1447253 | 16963 | 16982 | 2010 | 2029 | GGTCCTTCACTTGAGGGTCC | 98 | 334 |
| 1447258 | 18231 | 18250 | 3278 | 3297 | TTGAACAACGATTTGTGCTC | 40 | 335 |
| 1447263 | 17547 | 17566 | 2594 | 2613 | GGCGACCTCTTTCTCTGAAC | 44 | 336 |
| 1447268 | 5686 | 5705 | 1060 | 1079 | ACCTTATTATCTCCCAGCAT | 12† | 337 |
| 1447284 | 2034 | 2053 | N/A | N/A | GTGCCATCTCTTTTATAAGA | 111 | 338 |
| 1447295 | 1414 | 1433 | N/A | N/A | GAAGACATAATTATCCATGC | 74 | 339 |
| 1447299 | 4496 | 4515 | N/A | N/A | AAACGGTTTCCACTGTATGC | 59 | 340 |
| 1447300 | 12480 | 12499 | N/A | N/A | TGACCGCTTCTCTTTGGTAA | 71 | 341 |
| 1447305 | 18074 | 18093 | 3121 | 3140 | GAGCATTCAGACCCTTTCCC | 44 | 342 |
| 1447307 | 16282 | 16301 | N/A | N/A | TTCTGTTAACACCATGGACC | 71 | 343 |
| 1447308 | 17003 | 17022 | 2050 | 2069 | ATGTACACACCCAGCAAGCC | 59 | 344 |
| 1447312 | 3950 | 3969 | N/A | N/A | TGTTACCCTCAGATACCGCC | 16 | 345 |
| 1447330 | 16314 | 16333 | N/A | N/A | CACACTCCAAGCTAAGTCAC | 73 | 346 |
| 1447339 | 8005 | 8024 | N/A | N/A | GCAGGGTGCACACCAATTCC | 109 | 347 |
| 1447347 | 4129 | 4148 | N/A | N/A | TATCACCTTTAACTAGCTGT | 24 | 348 |
| 1447353 | 16503 | 16522 | N/A | N/A | CTTCACTGTCAAAATCTGGA | 92 | 349 |
| 1447354 | 10213 | 10232 | N/A | N/A | GCAAGGAGACCATTTACACA | 47 | 350 |
| 1447358 | 16322 | 16341 | N/A | N/A | GATTCTTCCACACTCCAAGC | 59 | 351 |
| 1447365 | 12589 | 12608 | N/A | N/A | TTCCTATAGAAGATTCATTC | 77 | 352 |
| 1447375 | 9299 | 9318 | N/A | N/A | GACACAGACATCGAGTTTTC | 52 | 353 |
| 1447379 | 18287 | 18306 | 3334 | 3353 | GTTTCTCTTCACCCCCAACA | 16 | 354 |
| 1447386 | 17080 | 17099 | 2127 | 2146 | GTAGCATCCAGATAAAATCA | 38 | 355 |
| 1447398 | 11793 | 11812 | N/A | N/A | AGATGAGCCTCCATACCTTC | 106 | 356 |
| 1447399 | 15489 | 15508 | N/A | N/A | ACCACCTTGTCCACTCACCT | 62 | 357 |
| 1447417 | 4552 | 4571 | N/A | N/A | CCTCACTGACTTCTTCAACT | 64 | 358 |
| 1447420 | 11836 | 11855 | N/A | N/A | ACTCTATGTACTTAATCAAT | 108 | 359 |
| 1447426 | 10335 | 10354 | N/A | N/A | ATCTTATGATGATCCTTTAT | 58 | 360 |
| 1447428 | 11199 | 11218 | N/A | N/A | CCTCACTAGTCCTCAGTATC | 62 | 361 |
| 1447429 | 14432 | 14451 | N/A | N/A | ACTCACCAGCTGCTTCTTTC | 154 | 362 |
| 1447435 | 17300 | 17319 | 2347 | 2366 | AGCTGCAAATCTTTCCCCTT | 33 | 363 |
| 1447454 | 14160 | 14179 | N/A | N/A | GCTTTTTCAACCCACAGGGA | 110 | 364 |
| 1447467 | 7864 | 7883 | N/A | N/A | TGGCACTGTTCCCTTCTAGT | 24 | 365 |
| 1447468 | 1043 | 1062 | N/A | N/A | TCCCAGTGAAAATCATGGCT | 72 | 366 |
| 1447477 | 17206 | 17225 | 2253 | 2272 | GACAGGTCAGACACAGGACT | 19 | 367 |
| 1447484 | 14969 | 14988 | N/A | N/A | AAGCTCAATTTACCCTATAC | 80 | 368 |
| 1447489 | 5817 | 5836 | N/A | N/A | GACGGTCTTTTTTCAAGAAC | 77 | 369 |
| 1447504 | 294 | 313 | N/A | N/A | ACCAAAGAAGATCTACCAAC | 91 | 370 |
| 1447505 | 11516 | 11535 | N/A | N/A | AAACCATGATCACACAAGGC | 54 | 371 |
| 1447507 | 4541 | 4560 | N/A | N/A | TCTTCAACTACTGTTTCTCC | 27 | 372 |
| 1447519 | 1539 | 1558 | N/A | N/A | AATGTCTGCTCACACCATTC | 107 | 373 |
| 1447521 | 2168 | 2187 | N/A | N/A | CAGACCACTTGAATATGTTT | 85 | 374 |
| 1447530 | 1820 | 1839 | N/A | N/A | GCGGAATTTATTACAGCTAG | 122 | 375 |
| 1447534 | 14035 | 14054 | N/A | N/A | ACAAGGCTAGACTTTCCCAC | 61 | 376 |
| 1447536 | 7499 | 7518 | N/A | N/A | CCATGGGTTTACCTCTCCTC | 62 | 377 |
| 1447541 | 11164 | 11183 | N/A | N/A | TGGATGAAATGACATCTAAT | 80 | 378 |
| 1447558 | 4914 | 4933 | N/A | N/A | CTGATTGCTAAAGACAGCAT | 40 | 379 |
| 1447559 | 17463 | 17482 | 2510 | 2529 | ATGGCAGCTAAGTCCCCTCA | 53 | 380 |
| 1447561 | 1202 | 1221 | N/A | N/A | GGTATGTACAAATTCTACAT | 71 | 381 |
| 1447566 | 4651 | 4670 | N/A | N/A | AGAGACTTTAATACCACCAT | 63 | 382 |
| 1447568 | 8111 | 8130 | N/A | N/A | GCATATCGATAGATTATCAT | 43 | 383 |
| 1447580 | 18100 | 18119 | 3147 | 3166 | GCGATCGCTTCTTCTCGCCA | 85 | 384 |
| 1447583 | 13790 | 13809 | N/A | N/A | TAACATTCACAGTCATGGTC | 57 | 385 |
| 1447585 | 8253 | 8272 | N/A | N/A | TCCGAAAAATCAATTCCATA | 36 | 386 |
| 1447589 | 5114 | 5133 | N/A | N/A | AAGGACTTCTTGCTCTTCCA | 20 | 387 |
| 1447590 | 5693 | 5712 | 1067 | 1086 | AGCTGGAACCTTATTATCTC | 52† | 388 |
| 1447597 | 9265 | 9284 | N/A | N/A | CCTACCAGATATGCAACCAC | 64 | 389 |
| 1447605 | 9148 | 9167 | N/A | N/A | GAGCTTTCCCCACCACATAC | 80 | 390 |
| 1447615 | 15186 | 15205 | N/A | N/A | CCTTTCTGTACCTCTTGGAT | 77 | 391 |
| 1447617 | 16684 | 16703 | N/A | N/A | GAAAACAGCTCCATACCTCC | 83 | 392 |
| 1447627 | 9601 | 9620 | N/A | N/A | GTATACAACCTATCAGTGGA | 52 | 393 |
| 1447628 | 3719 | 3738 | N/A | N/A | GGTTAATGAATCCTATCTCT | 34 | 394 |
| 1447633 | 685 | 704 | N/A | N/A | AGGTACAGATCTTACACCAT | 102 | 395 |
| 1447637 | 5901 | 5920 | N/A | N/A | TCCCACTAATGAATATTACA | 53 | 396 |
| 1447640 | 796 | 815 | N/A | N/A | CAGTCAACTGACCTTTCGAA | 71 | 397 |
| 1447644 | 32 | 51 | 32 | 51 | AGCAAGTTTATTGACCTGCC | 98 | 398 |
| 1447652 | 16922 | 16941 | 1969 | 1988 | TCCAATTGCCTTTTTGGAGA | 60 | 399 |

TABLE 6
Reduction of CHMP7 RNA by 5-10-5 MOE gapmers with mixed PO/PS internucleoside linkages in A431 cells

| | SEQ ID | SEQ | SEQ ID | SEQ | | | |
|---|---|---|---|---|---|---|---|
| | No: 1 | ID No: | No: 2 | ID No: | | CHMP7 | |
| Compound | Start | 1 Stop | Start | 2 Stop | | (% | SEQ ID |
| Number | Site | Site | Site | Site | Sequence (5′ to 3′) | UTC) | NO |
| 1447187 | 7543 | 7562 | N/A | N/A | GCAGGCTTTCTTTGTTTGCC | 59 | 400 |
| 1447192 | 1307 | 1326 | N/A | N/A | AGTCAATTCCCATATACCTA | 81 | 401 |
| 1447196 | 8002 | 8021 | N/A | N/A | GGGTGCACACCAATTCCATT | 84 | 402 |
| 1447200 | 14968 | 14987 | N/A | N/A | AGCTCAATTTACCCTATACC | 71 | 403 |
| 1447219 | 266 | 285 | N/A | N/A | AGTATATGATAATATGCCCA | 79 | 404 |
| 1447221 | 18072 | 18091 | 3119 | 3138 | GCATTCAGACCCTTTCCCGT | 57† | 405 |
| 1447225 | 5684 | 5703 | 1058 | 1077 | CTTATTATCTCCCAGCATGT | 13 | 406 |
| 1447232 | 12875 | 12894 | 1357 | 1376 | TGGTACACCCCAACATCTAC | 86 | 407 |
| 1447237 | 11982 | 12001 | N/A | N/A | AGGTCCCATCACACTACTCA | 65 | 408 |
| 1447250 | 17200 | 17219 | 2247 | 2266 | TCAGACACAGGACTGTATAA | 51 | 409 |
| 1447254 | 9051 | 9070 | N/A | N/A | TGCCTTCCTAAACAAGACCT | 86 | 410 |
| 1447269 | 15185 | 15204 | N/A | N/A | CTTTCTGTACCTCTTGGATC | 80 | 411 |
| 1447272 | 11515 | 11534 | N/A | N/A | AACCATGATCACACAAGGCA | 60 | 412 |
| 1447275 | 9532 | 9551 | N/A | N/A | GAAAGTCCTCAGCATATCCA | 60 | 413 |
| 1447278 | 16313 | 16332 | N/A | N/A | ACACTCCAAGCTAAGTCACC | 72 | 414 |
| 1447282 | 11792 | 11811 | N/A | N/A | GATGAGCCTCCATACCTTCT | 90 | 415 |
| 1447283 | 17299 | 17318 | 2346 | 2365 | GCTGCAAATCTTTCCCCTTC | 23 | 416 |
| 1447293 | 4650 | 4669 | N/A | N/A | GAGACTTTAATACCACCATA | 82 | 417 |
| 1447298 | 5454 | 5473 | N/A | N/A | TCATCACGACCATTTGTTAA | 78 | 418 |
| 1447301 | 17002 | 17021 | 2049 | 2068 | TGTACACACCCAGCAAGCCT | 92 | 419 |
| 1447302 | 9298 | 9317 | N/A | N/A | ACACAGACATCGAGTTTTCA | 78 | 420 |
| 1447304 | 5213 | 5232 | N/A | N/A | CGGTGAGATCTCCTTTTACA | 14 | 421 |
| 1447313 | 4536 | 4555 | N/A | N/A | AACTACTGTTTCTCCTGTTC | 34 | 422 |
| 1447318 | 17546 | 17565 | 2593 | 2612 | GCGACCTCTTTCTCTGAACA | 62 | 423 |
| 1447320 | 16528 | 16547 | 1789 | 1808 | AGGATGTCCAATTCCTTCTC | 111 | 424 |
| 1447321 | 17075 | 17094 | 2122 | 2141 | ATCCAGATAAAATCAGTGGT | 53 | 425 |
| 1447328 | 5815 | 5834 | N/A | N/A | CGGTCTTTTTTCAAGAACGC | 51 | 426 |
| 1447332 | 16657 | 16676 | 1918 | 1937 | AAGGACAGTTTCTCAAGTTC | 99 | 427 |
| 1447333 | 10655 | 10674 | N/A | N/A | TCACCATTTCGCTCATACAT | 42 | 428 |
| 1447350 | 18099 | 18118 | 3146 | 3165 | CGATCGCTTCTTCTCGCCAT | 71 | 429 |
| 1447352 | 4549 | 4568 | N/A | N/A | CACTGACTTCTTCAACTACT | 58 | 430 |
| 1447357 | 4903 | 4922 | N/A | N/A | AGACAGCATAAAATTTGTGC | 40 | 431 |
| 1447367 | 16277 | 16296 | N/A | N/A | TTAACACCATGGACCTGACC | 70 | 432 |
| 1447374 | 11249 | 11268 | N/A | N/A | GCTGAGGCCTGATTTCCAGC | 91 | 433 |
| 1447377 | 13097 | 13116 | N/A | N/A | GCTTTCCATGATTTCTGCAT | 73 | 434 |
| 1447378 | 5862 | 5881 | N/A | N/A | AGCAGATAAACTCAGTTATC | 45 | 435 |
| 1447380 | 17420 | 17439 | 2467 | 2486 | TCTCATTCTTACCAGTGAAA | 31 | 436 |
| 1447385 | 16425 | 16444 | N/A | N/A | GACTGAGGTTACCAAAACCC | 49 | 437 |
| 1447401 | 10332 | 10351 | N/A | N/A | TTATGATGATCCTTTATTCT | 65 | 438 |
| 1447402 | 1197 | 1216 | N/A | N/A | GTACAAATTCTACATAAGGA | 106 | 439 |
| 1447406 | 5734 | 5753 | N/A | N/A | GTACCCACCTTCAACAGCTC | 81† | 440 |
| 1447409 | 1817 | 1836 | N/A | N/A | GAATTTATTACAGCTAGGCA | 113 | 441 |
| 1447414 | 16321 | 16340 | N/A | N/A | ATTCTTCCACACTCCAAGCT | 70 | 442 |
| 1447418 | 14422 | 14441 | 1465 | 1484 | TGCTTCTTTCCTGCTCGGCA | 65 | 443 |
| 1447437 | 2165 | 2184 | N/A | N/A | ACCACTTGAATATGTTTATA | 101 | 444 |
| 1447443 | 27 | 46 | 27 | 46 | GTTTATTGACCTGCCGGCCT | 111 | 445 |
| 1447456 | 16805 | 16824 | N/A | N/A | AGTCTTAATATAAACAACCC | 110 | 446 |
| 1447457 | 927 | 946 | N/A | N/A | GGACGGATACAACCCAACTT | 75 | 447 |
| 1447474 | 8109 | 8128 | N/A | N/A | ATATCGATAGATTATCATGC | 43 | 448 |
| 1447480 | 18279 | 18298 | 3326 | 3345 | TCACCCCCAACATTTCCGTT | 61 | 449 |
| 1447492 | 14030 | 14049 | N/A | N/A | GCTAGACTTTCCCACCTGGA | 58 | 450 |
| 1447494 | 18207 | 18226 | 3254 | 3273 | CCTGGAGCAAATTCTTCTCC | 81 | 451 |
| 1447506 | 12549 | 12568 | N/A | N/A | CTCAGCAAATGATATTTGGT | 55 | 452 |
| 1447538 | 684 | 703 | N/A | N/A | GGTACAGATCTTACACCATT | 108 | 453 |
| 1447539 | 5692 | 5711 | 1066 | 1085 | GCTGGAACCTTATTATCTCC | 11† | 454 |
| 1447543 | 2033 | 2052 | N/A | N/A | TGCCATCTCTTTTATAAGAT | 120 | 455 |
| 1447545 | 8220 | 8239 | N/A | N/A | AAGTACATAGATCTCTGCAC | 60 | 456 |
| 1447546 | 5110 | 5129 | N/A | N/A | ACTTCTTGCTCTTCCAAGGC | 23 | 457 |
| 1447552 | 14125 | 14144 | N/A | N/A | GGGCACATTATACACATCTT | 60 | 458 |
| 1447557 | 3712 | 3731 | N/A | N/A | GAATCCTATCTCTTAGACCT | 59 | 459 |
| 1447569 | 10068 | 10087 | N/A | N/A | GTGGAAAGTCACTATGGATT | 46 | 460 |
| 1447573 | 11156 | 11175 | N/A | N/A | ATGACATCTAATTCCCTTCC | 46 | 461 |
| 1447577 | 7021 | 7040 | N/A | N/A | GTGCTCAGTCAACACACACA | 75 | 462 |
| 1447582 | 15488 | 15507 | N/A | N/A | CCACCTTGTCCACTCACCTA | 73 | 463 |
| 1447604 | 5407 | 5426 | N/A | N/A | GCATTGTTTATACTCCAGCT | 2 | 464 |
| 1447606 | 4247 | 4266 | N/A | N/A | CATCAGAGTTGACTTTCTCC | 35 | 465 |
| 1447609 | 3909 | 3928 | N/A | N/A | GTATCTCCTACAGACCTTTT | 28 | 466 |
| 1447611 | 1534 | 1553 | N/A | N/A | CTGCTCACACCATTCCTAGT | 84 | 467 |
| 1447613 | 12520 | 12539 | N/A | N/A | TTGGTCCAGAACTTTTACCT | 69 | 468 |
| 1447618 | 16953 | 16972 | 2000 | 2019 | TTGAGGGTCCTACAATGGCT | 43 | 469 |
| 1447620 | 11197 | 11216 | N/A | N/A | TCACTAGTCCTCAGTATCAC | 69 | 470 |
| 1447621 | 16328 | 16347 | N/A | N/A | AGAGATGATTCTTCCACACT | 67 | 471 |
| 1447625 | 9247 | 9266 | N/A | N/A | ACTGCAGTAACTGCACAGTA | 111 | 472 |
| 1447631 | 11831 | 11850 | N/A | N/A | ATGTACTTAATCAATCACTG | 73 | 473 |
| 1447634 | 4494 | 4513 | N/A | N/A | ACGGTTTCCACTGTATGCCA | 33 | 474 |
| 1447641 | 749 | 768 | N/A | N/A | GAGTCTTTTCAACAATGAGC | 82 | 475 |
| 1447649 | 14395 | 14414 | 1438 | 1457 | CGGGCTTCTTCTTTACACCT | 76 | 476 |
| 1447651 | 4053 | 4072 | N/A | N/A | TGAGGATAATTCTATCACCT | 12 | 477 |

### Example 2: Dose-Dependent Inhibition of Human CHMP7 in A431 Cells by Modified Oligonucleotides

Modified oligonucleotides selected from Example 1 above were tested at various doses in A431 cells. Cultured A431 cells at a density of 10,000 cells per well were treated by free uptake with various concentrations of modified oligonucleotide as specified in the tables below. After a treatment period of approximately 48 hours, total RNA was isolated from the cells and CHMP7 RNA levels were measured by quantitative real-time RTPCR Human CHMP7 primer-probe set RTS50844 (described herein in Example 1) was used to measure RNA levels as described above. CHMP7 RNA levels were normalized to total RNA content, as measured by RIBOGREEN®. Reduction of CHMP7 RNA is presented in the tables below as percent CHMP7 RNA, relative to untreated control cells (% UTC). Modified oligonucleotides marked with an “T” indicate that the modified oligonucleotide is complementary to the amplicon region of the primer probe set. Additional assays may be used to measure the potency and efficacy of the modified oligonucleotides complementary to the amplicon region.

The half maximal inhibitory concentration (IC₅₀) of each modified oligonucleotide was calculated using a linear regression on a log/linear plot of the data in Excel and is also presented in the tables below.

TABLE 7
Dose-dependent reduction of human CHMP7 RNA
in A431 cells by modified oligonucleotides

| | CHMP7 RNA (% UTC) | | | | |
|---|---|---|---|---|---|
| Compound | 62.5 | 250.0 | 1,000.0 | 4,000.0 | IC₅₀ |
| No. | nM | nM | nM | nM | (μM) |
| 1447190 | 75 | 48 | 22 | 9 | 0.25 |
| 1447260† | 86 | 79 | 56 | 26 | 1.13 |
| 1447309 | 55 | 26 | 9 | 5 | 0.06 |
| 1447314 | 56 | 25 | 11 | 7 | 0.06 |
| 1447315 | 60 | 28 | 10 | 3 | 0.08 |
| 1447326 | 75 | 29 | 16 | 8 | 0.16 |
| 1447431 | 37 | 13 | 4 | 1 | <0.0625 |
| 1447449 | 78 | 51 | 25 | 11 | 0.29 |
| 1447471 | 60 | 26 | 9 | 6 | 0.07 |
| 1447481 | 56 | 24 | 14 | 6 | 0.06 |
| 1447488 | 70 | 28 | 12 | 4 | 0.13 |
| 1447497 | 54 | 28 | 15 | 8 | 0.06 |
| 1447502† | 96 | 76 | 58 | 38 | 1.72 |
| 1447520 | 61 | 30 | 15 | 9 | 0.09 |
| 1447532 | 86 | 48 | 33 | 16 | 0.39 |
| 1447555† | 70 | 45 | 19 | 4 | 0.19 |
| 1447602 | 64 | 30 | 10 | 4 | 0.10 |
| 1447624 | 75 | 45 | 22 | 9 | 0.24 |

TABLE 8
Dose-dependent reduction of human CHMP7 RNA
in A431 cells by modified oligonucleotides

| | CHMP7 RNA (% UTC) | | | | |
|---|---|---|---|---|---|
| Compound | 62.5 | 250.0 | 1,000.0 | 4,000.0 | IC₅₀ |
| No. | nM | nM | nM | nM | (μM) |
| 1447218† | 87 | 55 | 29 | 12 | 0.40 |
| 1447228 | 80 | 40 | 23 | 9 | 0.24 |
| 1447233 | 80 | 70 | 44 | 22 | 0.65 |
| 1447256 | 64 | 37 | 16 | 6 | 0.13 |
| 1447273 | 85 | 51 | 34 | 19 | 0.42 |
| 1447279 | 110 | 62 | 30 | 18 | 0.63 |
| 1447285 | 83 | 61 | 40 | 29 | 0.65 |
| 1447331 | 55 | 26 | 8 | 2 | 0.06 |
| 1447364 | 81 | 56 | 28 | 11 | 0.36 |
| 1447369 | 63 | 42 | 21 | 14 | 0.15 |
| 1447370 | 78 | 57 | 29 | 12 | 0.34 |
| 1447421 | 104 | 76 | 49 | 33 | 1.22 |
| 1447433 | 105 | 86 | 62 | 45 | 2.60 |
| 1447535 | 94 | 42 | 18 | 7 | 0.31 |
| 1447549 | 62 | 42 | 21 | 5 | 0.14 |
| 1447560 | 74 | 43 | 14 | 8 | 0.20 |
| 1447567 | 79 | 47 | 28 | 13 | 0.30 |
| 1447595 | 26 | 5 | 1 | 1 | <0.0625 |

TABLE 9
Dose-dependent reduction of human CHMP7 RNA
in A431 cells by modified oligonucleotides

| | CHMP7 RNA (% UTC) | | | | |
|---|---|---|---|---|---|
| Compound | 62.5 | 250.0 | 1,000.0 | 4,000.0 | IC₅₀ |
| No. | nM | nM | nM | nM | (μM) |
| 1447224 | 70 | 39 | 14 | 3 | 0.16 |
| 1447225† | 84 | 64 | 33 | 17 | 0.49 |
| 1447236 | 33 | 7 | 1 | 0 | <0.0625 |
| 1447248 | 64 | 41 | 24 | 13 | 0.15 |
| 1447268† | 90 | 60 | 26 | 10 | 0.42 |
| 1447283 | 79 | 42 | 22 | 13 | 0.25 |
| 1447304 | 58 | 26 | 14 | 7 | 0.07 |
| 1447312 | 99 | 55 | 28 | 14 | 0.48 |
| 1447343 | 74 | 57 | 34 | 16 | 0.36 |
| 1447347 | 64 | 43 | 23 | 10 | 0.16 |
| 1447379 | 79 | 41 | 18 | 5 | 0.23 |
| 1447467 | 82 | 44 | 26 | 19 | 0.31 |
| 1447477 | 76 | 54 | 27 | 9 | 0.30 |
| 1447539† | 92 | 67 | 71 | 31 | 1.64 |
| 1447546 | 68 | 34 | 16 | 6 | 0.14 |
| 1447589 | 61 | 33 | 18 | 8 | 0.10 |
| 1447604 | 26 | 7 | 2 | 1 | <0.0625 |
| 1447651 | 84 | 56 | 18 | 8 | 0.32 |

### Example 3: Design of 5-10-5 MOE Gapmer Modified Oligonucleotides with PS Internucleoside Linkages Complementary to a Human CHMP7 Nucleic Acid

Modified oligonucleotides complementary to human CHMP7 nucleic acid were designed and synthesized. “Start site” in all the tables below indicates the 5′-most nucleoside of the target sequence to which the modified oligonucleotide is complementary. “Stop site” in all the tables below indicates the 3′-most nucleoside of the target sequence to which the modified oligonucleotide is complementary. As shown in the tables below, the modified oligonucleotides are complementary to either SEQ ID NO: 1 (described herein above), and/or to SEQ ID NO: 2 (described herein above). ‘N/A’ indicates that the modified oligonucleotide is not complementary to that particular target sequence with 100% complementarity.

The modified oligonucleotides in the table below are 5-10-5 MOE gapmers. The sugar motif of the gapmers is (from 5′ to 3′): eeeeeddddddddddeeeee; wherein ‘d’ represents a 2′-β-D-deoxyribosyl sugar moiety, and ‘e’ represents a 2′-MOE modified sugar moiety. The internucleoside motif of the gapmers is (from 5′ to 3′): sssssssssssssssssss, wherein each “s” represents a phosphorothioate internucleoside linkage. Each cytosine nucleoside is a 5-methyl cytosine.

TABLE 10
5-10-5 MOE gapmers PS internucleoside linkages complementary to human CHMP7

| | | SEQ | SEQ | SEQ | SEQ | |
|---|---|---|---|---|---|---|
| | | ID | ID | ID | ID | |
| Com- | | No: 1 | No: 1 | No: 2 | No: 2 | SEQ |
| pound | | Start | Stop | Start | Stop | ID |
| No. | Sequence (5′ to 3′) | Site | Site | Site | Site | No. |
| 1508916 | GAAAACGGTTTCCACTGTAT | 4498 | 4517 | N/A | N/A | 257 |
| 1508917 | TGTTACCCTCAGATACCGCC | 3950 | 3969 | N/A | N/A | 345 |
| 1508918 | ATGTGATGCTATTAATAGGA | 10035 | 10054 | N/A | N/A | 12 |

### Example 4: Dose-Dependent Inhibition of Human CHMP7 in A431 Cells by Modified Oligonucleotides

Modified oligonucleotides described in Example 3 above were tested at various doses in A431 cells. Cultured A431 cells at a density of 10,000 cells per well were treated by free uptake with various concentrations of modified oligonucleotide as specified in the tables below. After a treatment period of approximately 48 hours, total RNA was isolated from the cells and CHMP7 RNA levels were measured by quantitative real-time RTPCR Human CHMP7 primer-probe set RTS50844 (described herein in Example 1) was used to measure RNA levels as described above. CHMP7 RNA levels were normalized to total RNA content, as measured by RIBOGREEN®. Reduction of CHMP7 RNA is presented in the tables below as percent CHMP7 RNA, relative to untreated control cells (% UTC).

The half maximal inhibitory concentration (IC₅₀) of each modified oligonucleotide was calculated using a linear regression on a log/linear plot of the data in Excel and is also presented in the tables below.

TABLE 11
Dose-dependent reduction of human CHMP7 RNA in A431 cells by modified oligonucleotides

| | CHMP7 RNA (% UTC) | | | | | | | | | | |
|---|---|---|---|---|---|---|---|---|---|---|---|
| Compound | 0.02 | 0.06 | 0.24 | 0.98 | 3.91 | 15.63 | 62.5 | 250.0 | 1000.0 | 4000.0 | IC₅₀ |
| No. | nM | nM | nM | nM | nM | nM | nM | nM | nM | nM | (μM) |
| 1508916 | 121 | 107 | 106 | 107 | 104 | 99 | 73 | 51 | 28 | 18 | 0.37 |
| 1508917 | 96 | 97 | 93 | 91 | 91 | 77 | 40 | 18 | 6 | 3 | 0.03 |
| 1508918 | 110 | 98 | 103 | 101 | 112 | 105 | 89 | 70 | 39 | 27 | 2.96 |

1. An oligomeric compound comprising a modified oligonucleotide consisting of 12 to 50 linked nucleosides wherein the nucleobase sequence of the modified oligonucleotide is at least 90% complementary to an equal length portion of a CHMP7 nucleic acid, and wherein the modified oligonucleotide comprises at least one modification selected from a modified sugar moiety and a modified internucleoside linkage.

2. An oligomeric compound comprising a modified oligonucleotide consisting of 12 to 50 linked nucleosides and having a nucleobase sequence comprising at least 12, at least 13, at least 14, at least 15, at least 16, at least 17, at least 18, at least 19, or 20 contiguous nucleobases of any of the nucleobases of SEQ ID NOs: 10-477, wherein the modified oligonucleotide comprises at least one modification selected from a modified sugar moiety and a modified internucleoside linkage.

3. The oligomeric compound of claim 2, wherein the modified oligonucleotide has a nucleobase sequence consisting of the nucleobase sequence of any of SEQ ID NOs: 10-477.

4. An oligomeric compound comprising a modified oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19, or at least 20 contiguous nucleobases complementary to:
an equal length portion within nucleobases 3950-3983 of SEQ ID NO: 1;
an equal length portion within nucleobases 4242-4266 of SEQ ID NO: 1;
an equal length portion within nucleobases 4480-4525 of SEQ ID NO: 1;
an equal length portion within nucleobases 4534-4566 of SEQ ID NO: 1;
an equal length portion within nucleobases 5205-5232 of SEQ ID NO: 1;
an equal length portion within nucleobases 5404-5430 of SEQ ID NO: 1;
an equal length portion within nucleobases 8323-8344 of SEQ ID NO: 1;
an equal length portion within nucleobases 16927-16950 of SEQ ID NO: 1;
an equal length portion within nucleobases 17298-17340 of SEQ ID NO: 1; or
an equal length portion within nucleobases 18287-18313 of SEQ ID NO: 1;
wherein the modified oligonucleotide comprises at least one modification selected from a modified sugar moiety and a modified internucleoside linkage.

5. An oligomeric compound comprising a modified oligonucleotide consisting of 12 to 30 linked nucleosides and having a nucleobase sequence comprising at least 8, at least 9, at least 10, at least 11, at least 12, at least 13, at least 14, at least 15, at least 16, at least 17, or at least 18 contiguous nucleobases of a sequence selected from:
SEQ ID NOs: 220, 302, and 345;
SEQ ID NOs: 21, 131, 191, and 465;
SEQ ID NOs: 34, 116, 184, 242, 257, 340, and 474;
SEQ ID NOs: 55, 118, 202, 267, 372, and 422;
SEQ ID NOs: 73, 136, 197, and 421;
SEQ ID NOs: 79, 160, 168, 230, 313, 331, and 464;
SEQ ID NOs: 157, 186, and 265;
SEQ ID NOs: 128, 182, and 309;
SEQ ID NOs: 44, 76, 153, 206, 283, 363, and 416; or
SEQ ID NOs: 85, 121, 189, 300, and 354;
wherein the modified oligonucleotide comprises at least one modification selected from a modified sugar moiety and a modified internucleoside linkage.

6. The oligomeric compound of any of claims 1-5, wherein the modified oligonucleotide has a nucleobase sequence that is at least 80%, 85%, 90%, 95%, or 100% complementary to the nucleobase sequence of SEQ ID NO: 1 or SEQ ID NO: 2 when measured across the entire nucleobase sequence of the modified oligonucleotide.

7. The oligomeric compound of any of claims 1-6, wherein the modified oligonucleotide comprises at least one modified nucleoside.

8. The oligomeric compound of claim 7, wherein the modified oligonucleotide comprises at least one modified nucleoside comprising a modified sugar moiety.

9. The oligomeric compound of claim 8, wherein the modified oligonucleotide comprises at least one modified nucleoside comprising a bicyclic sugar moiety.

10. The oligomeric compound of claim 9, wherein the modified oligonucleotide comprises at least one modified nucleoside comprising a bicyclic sugar moiety having a 2′-4′ bridge, wherein the 2′-4′ bridge is selected from —O—CH₂—; and —O—CH(CH₃)—.

11. The oligomeric compound of any of claims 7-10, wherein the modified oligonucleotide comprises at least one modified nucleoside comprising a non-bicyclic modified sugar moiety.

12. The oligomeric compound of claim 11, wherein the modified oligonucleotide comprises at least one modified nucleoside comprising a non-bicyclic modified sugar moiety comprising a 2′-MOE modified sugar moiety or a 2′-OMe modified sugar moiety.

13. The oligomeric compound of any of claims 7-12, wherein the modified oligonucleotide comprises at least one modified nucleoside comprising a sugar surrogate.

14. The oligomeric compound of claim 13, wherein the sugar surrogate is selected from any of morpholino, modified morpholino, PNA, THP, and F-HNA.

15. The oligomeric compound of any of claim 1-8 or 11-14, wherein the modified oligonucleotide does not comprise a bicyclic modified sugar moiety.

16. The oligomeric compound of any of claims 1-15, wherein the modified oligonucleotide is a gapmer.

17. The oligomeric compound of any of claims 1-16 wherein the modified oligonucleotide comprises a deoxy region consisting of 5-12 linked 2′-deoxynucleosides.

18. The oligomeric compound of any of claims 1-16, wherein the modified oligonucleotide comprises a deoxy region consisting of 5-12 linked 2′-β-D-deoxynucleosides.

19. The oligomeric compound of claim 17 or claim 18, wherein the deoxy region consists of 6, 7, 8, 9, 10, or 6-10 linked nucleosides.

20. The oligomeric compound of any of claims 17-19, wherein each nucleoside immediately adjacent to the deoxy region comprises a modified sugar moiety.

21. The oligomeric compound of any of claims 17-20, wherein the deoxy region is flanked on the 5′-side by a 5′-external region consisting of 1-6 linked 5′-external region nucleosides and on the 3′-side by a 3′-external region consisting of 1-6 linked 3′-external region nucleosides; wherein
the 3′-most nucleoside of the 5′ external region comprises a modified sugar moiety; and
the 5′-most nucleoside of the 3′ external region comprises a modified sugar moiety.

22. The oligomeric compound of claim 21, wherein each nucleoside of the 3′ external region comprises a modified sugar moiety.

23. The oligomeric compound of claim 21 or claim 22, wherein each nucleoside of the 5′ external region comprises a modified sugar moiety.

24. The oligomeric compound of any of claims 1-23, wherein the modified oligonucleotide comprises:
a 5′-region consisting of 1-7 linked 5′-region nucleosides;
a central region consisting of 6-10 linked central region nucleosides; and
a 3′-region consisting of 1-7 linked 3′-region nucleosides; wherein
each of the 5′-region nucleosides and each of the 3′-region nucleosides comprises a modified sugar moiety and each of the central region nucleosides comprises a 2′-β-D-deoxyfuranosyl sugar moiety.

25. The oligomeric compound of claim 24, wherein the modified oligonucleotide comprises:
a 5′-region consisting of 5 linked 5′-region nucleosides;
a central region consisting of 10 linked central region nucleosides; and
a 3′-region consisting of 5 linked 3′-region nucleosides; wherein
each of the 5′-region nucleosides and each of the 3′-region nucleosides is a 2′-MOE nucleoside and each of the central region nucleosides is a 2′-β-D-deoxynucleoside.

26. The oligomeric compound of any of claims 1-25, wherein the modified oligonucleotide comprises at least one modified internucleoside linkage.

27. The oligomeric compound of claim 26, wherein each internucleoside linkage of the modified oligonucleotide is a modified internucleoside linkage.

28. The oligomeric compound of claim 26 or claim 27 wherein at least one modified internucleoside linkage is a phosphorothioate internucleoside linkage.

29. The oligomeric compound of claim 26 or claim 28 wherein the modified oligonucleotide comprises at least one phosphodiester internucleoside linkage.

30. The oligomeric compound of any of claim 26, 28, or 29, wherein each internucleoside linkage is either a phosphodiester internucleoside linkage or a phosphorothioate internucleoside linkage.

31. The oligomeric compound of claim 27, wherein each modified internucleoside linkage is a phosphorothioate internucleoside linkage

32. The oligonucleotide compound of claim 26, wherein the modified oligonucleotide has an internucleoside linkage motif of soooossssssssssooss; wherein,
s=a phosphorothioate internucleoside linkage and o=a phosphodiester internucleoside linkage.

33. The oligomeric compound of any of claims 1-32, wherein the modified oligonucleotide comprises at least one modified nucleobase.

34. The oligomeric compound of claim 33, wherein the modified nucleobase is a 5-methyl cytosine.

35. The oligomeric compound of claim 34, wherein each cytosine is a 5-methyl cytosine.

36. The oligomeric compound of any of claims 1-35, wherein the modified oligonucleotide consists of 12-30, 12-22, 12-20, 14-18, 14-20, 15-17, 15-25, 16-18, 16-20, 17-20, 18-20 or 18-22 linked nucleosides.

37. The oligomeric compound of any of claims 1-36, wherein the modified oligonucleotide consists of 16, 17, 18, 19, or 20 linked nucleosides.

38. The oligomeric compound of any of claims 1-35, wherein the modified oligonucleotide consists of 20 linked nucleosides.

39. The oligomeric compound of any of claims 1-38, consisting of the modified oligonucleotide.

40. The oligomeric compound of any of claims 1-38, wherein the oligomeric compound comprises a conjugate group.

41. The oligomeric compound of claim 40, wherein the conjugate group comprises a conjugate moiety and a conjugate linker.

42. The oligomeric compound of claim 41, wherein the conjugate linker consists of a single bond.

43. The oligomeric compound of claim 41 or claim 42, wherein the conjugate linker is cleavable.

44. The oligomeric compound of claim 41 or claim 43, wherein the conjugate linker comprises 1-3 linker-nucleosides.

45. The oligomeric compound of any of claims 41-43, wherein the conjugate linker does not comprise any linker nucleosides.

46. The oligomeric compound of any of claims 40-45, wherein the conjugate group is attached to the modified oligonucleotide at the 5′-end of the modified oligonucleotide.

47. The oligomeric compound of any of claims 40-45, wherein the conjugate group is attached to the modified oligonucleotide at the 3′-end of the modified oligonucleotide.

48. The oligomeric compound of any of claims 40-47, wherein the conjugate group comprises a lipid.

49. The oligomeric compound of any of claims 40-47, wherein the conjugate group comprises a cell-targeting moiety.

50. The oligomeric compound of any of claims 1-49, further comprising a terminal group.

51. The oligomeric compound of any of claims 1-49, wherein the oligomeric compound is a singled-stranded oligomeric compound.

52. The oligomeric compound of any of claims 1-51, wherein the oligomeric compound is capable of reducing the amount of CHMP7 RNA in a cell.

53. The oligomeric compound of any of claims 1-52, wherein the modified oligonucleotide of the oligomeric compound is a pharmaceutically acceptable salt comprising one or more cations selected from sodium, potassium, calcium, and magnesium.

54. An oligomeric duplex, comprising a first oligomeric compound and a second oligomeric compound comprising a second modified oligonucleotide, wherein the first oligomeric compound is an oligomeric compound of any of claims 1-53.

55. An antisense agent comprising an antisense compound, wherein the antisense compound is the oligomeric compound of any of claims 1-53 or the oligomeric duplex of claim 54.

56. The antisense agent of claim 55, wherein the antisense agent is the oligomeric duplex of claim 54

57. The antisense agent of claim 55 or claim 56, wherein the antisense agent is:
an RNase H agent capable of reducing the amount of CHMP7 nucleic acid through the activation of RNase H; or
an RNAi agent capable of reducing the amount of CHMP7 nucleic acid through the activation of RISC/Ago2.

58. The antisense agent of any of claims 55-57, wherein the antisense agent comprises a conjugate group, wherein the conjugate group comprises a cell-targeting moiety.

59. A pharmaceutical composition comprising the oligomeric compound of any of claims 1-53, the oligomeric duplex of claim 54, or the antisense agent of any of claims 55-58, and a pharmaceutically acceptable diluent.

60. The pharmaceutical composition of claim 59, wherein the pharmaceutically acceptable diluent is artificial CSF (aCSF) or phosphate-buffered saline (PBS).

61. The pharmaceutical composition of claim 60, wherein the pharmaceutical composition consists essentially of the oligomeric compound, oligomeric duplex, or antisense agent, and artificial CSF (aCSF).

62. The pharmaceutical composition of claim 60, wherein the pharmaceutical composition consists essentially of the oligomeric compound, oligomeric duplex, or antisense agent, and phosphate buffered saline (PBS).

63. A chirally enriched population of oligomeric compounds of any of claims 1-53, wherein the population is enriched for oligomeric compounds comprising at least one particular phosphorothioate internucleoside linkage having a particular stereochemical configuration.

64. The chirally enriched population of claim 63, wherein the population is enriched for oligomeric compounds comprising at least one particular phosphorothioate internucleoside linkage having the (Sp) configuration.

65. The chirally enriched population of claim 63, wherein the population is enriched for oligomeric compounds comprising at least one particular phosphorothioate internucleoside linkage having the (Rp) configuration.

66. The chirally enriched population of claim 63, wherein the population is enriched for oligomeric compounds having a particular, independently selected stereochemical configuration at each phosphorothioate internucleoside linkage.

67. The chirally enriched population of claim 66, wherein the population is enriched for oligomeric compounds having the (Sp) configuration at each phosphorothioate internucleoside linkage or for modified oligonucleotides having the (Rp) configuration at each phosphorothioate internucleoside linkage.

68. The chirally enriched population of claim 66, wherein the population is enriched for oligomeric compounds having the (Rp) configuration at one particular phosphorothioate internucleoside linkage and the (Sp) configuration at each of the remaining phosphorothioate internucleoside linkages.

69. The chirally enriched population of claim 66, wherein the population is enriched for oligomeric compounds having at least 3 contiguous phosphorothioate internucleoside linkages in the Sp, Sp, and Rp configurations, in the 5′ to 3′ direction.

70. A population of oligomeric compounds of any of claims 1-53, wherein all of the phosphorothioate internucleoside linkages of the modified oligonucleotide are stereorandom.

71. A pharmaceutical composition comprising the population of oligomeric compounds of any of claims 63-70 and a pharmaceutically acceptable diluent.

72. The pharmaceutical composition of claim 71, wherein the pharmaceutically acceptable diluent is artificial CSF (aCSF) or phosphate-buffered saline (PBS).

73. The pharmaceutical composition of claim 72, wherein the pharmaceutical composition consists essentially of the population of oligomeric compounds and artificial CSF (aCSF).

74. The pharmaceutical composition of claim 72, wherein the pharmaceutical composition consists essentially of the population of oligomeric compounds and PBS.


SEQUENCE LISTING (477 sequences)

SEQ ID NO: 1 - DNA, 18364 bp, Homo sapiens
gctgcctcctctgactgttatgcagcaggccggcaggtcaataaacttgcttgcctgactttgagtatactcgtcttttctcttggccaaccttacggtagctgtaccagtttgcatttccaccagcaatgaatgagagttcctgttgctctgcatcctcaccagcatttggtgtggtcagtattttggattttcaccattctaataggtgtgtagtagtatgctattcttgtctaatttgctgttccccaatgacatatgatgttgggcatattatcatatacttatttgccgttggtagatcttctttggtgagaggtctgctcagatcttttatccatgttttaactgggttattctttttcttttttttttgagtcttaagagttcctcgtgtgttttggataacagtcctttatcagagatgtcttttgcaaatagtttctaccagtctgtggcttatcttcacattctcttcacagtgtctttcagaaagcaaataataataataataataattacgtctaatttatcaattatttctttcatggattatgctttttgtgttgtatctaaaaagtaatcatgatgcccaaggtcatctagatgttttgtcttctaggagttttattgttttgtgtttttacatttaagtccatgatccatttgagctggcttttgtgaatggtgtaagatctgtacctggatttgtttttgtttttgcatgtaggtgtacagttgttccagtgctcattgttgaaaagactctttgaaccattgtattgcctttcttttttcgaaaggtcagttgactgtatttacatgtgcctatttctgggattcctattctgttccatttatctatttgtttattctttaactaataccacactatcttgattactgttgatttatagtaattcttgaagttgggttgtatccgtcctctgacttatttgttcaatattgtgttggctcttctgggtcttttgcctttccacataaactttagaattagtttgttgatatccacaaaataactagccatgattttcactgggatttcactggatctacagatcaatttgggaagaattgatatcttgataatattgagttttcctgtccatgaatataaaatgcctctgtatttatttagtccttcatagatttctttcatcagaattttgtagttttccttatgtagaatttgtacataccgtgttagatttatacctaagttttataggggggtgctaatgtaaatggtattgcatttttaatttcatattccacttgatcattataggtatatgggaattgacttttatacattaactttgtatctgcaatcttgttctcattgcctcttagttccaggacatcgatttggcaattctttcataattttctgcatggataattatgtcttctataaagacagaccattttatttcttcctcttcagtctgtacaacttttatttctttttcttggctaactgtgttagctaggatttcccgtacagtgttgactaggaatggtgtgagcagacattcttgtgttattcctgattccactggaaaacgatctaggttttcaccattaattcttatgttggctgtaaggttcctgtttatgttctttatcaagtggaggatgtgtccctctattcctagagtgccgagactttttatcatgagctggtgttggattttgtcaaatgctttttctgcatctagcaatatgatcatatgatttttctttagtctgttggtggattatattaattgatttttgaacattgaactagccttgcctagctgtaataaattccgctttaacctggtgtacaattcttttgatacattgtgggtttcaatttgctaacattctattgatgatttttgcatctatattcacaagatatattgttttgtgttttttctttcttgtaatgtctttgtgtggttttggcgttagggtaatccttgcctcataggatcagttgggaagtattccttttgtttctatcttataaaagagatggcacagaactggtacaacttttttcttaaatatttggtagaacttatcaaatgaaaccatctgggcctaatgctttctgtgtggatgttgttaattattgattcaattaattagatataaacatattcaagtggtctgtttcctcttgtgtgttttggcagagtgtgctttcaagaaatgtgtccatttcgtctaggttatcaaatttgtggacatacagttattcatagtattcctttattatcattttcatgcccatgggatctgcagtaatgtcccttatttcatttctgatattgagtatttgttttttctctttgttagccaggctagaggttaatcaatttcattgaccttttcaaaaaatcagctttaggttttgttgataatcttttgagtttgttttcaatttcattgatttctgctctttttattattttttctcttctgtttaatttgatcttaatttgttcttcttttatagattcccaaggtggaatcttaggttattttagatctttcttcttttctggtttgtgcattcattgatataaatttccctctatacagagattcacggatccatccgggtggtccgggcaccactggcgcccctctgcggctattccccagttccattttctgaagccacaggtcagaagccgctgtacaacgctttgccgaactccagaatcttgaaggagacgtaaggtgcagccacctgccgcgcaggcgcaagcctttctttcggcacaaagaccgtgggaggaggggtcggcgcaagcgctcggtgtctctctgaaaagaacttcatcatactgcctcctggctgacggagcgcagcgcaacgcatgcgccttgaagacttatggaacttatttcacgctcagggcggcggtgacgtgtgaacgagaaggaggtggtcaaggaacggaagccgggagggaacgagggcggaagcggaccagggccaggcttgtgttcgcagccttgccggggctggggttccgatgtggtccccggagcgggaggccgaggccccagccgggggagacccggcgggccttctgccccccgagtgggaggaggacgaggagcgcatgtccttcctgttctccgctttcaagaggagtcgcgaggtgaacagcaccgactgggacagcaagatgggcttctgggcgccgttggtgctgagccacagccgccgccagggggtggtgcgcctgcgtctgcgggacttgcaggaggcctttcagcgcaaggggagcgtcccgctggggctggccacggtgctgcaggacctgctgcggtgaggggcgggctggggccagggccgcgagggcgggcgggggcagctctcggagggccgggccctggtcctgttctctgcacagcgcactgcgcccagcccagtgtctggcccagagaaggcatccagtgtgtgttcagaaggcgaagaaggaaaataaggaggctgggctagggaggcttggagggagaggaagaggggatctgggggaagattggggggcggggatagtccgattagacgagttccagagaagccagcctaacagaggtgacagtaatttgcctggactgggaccccgttatgttgttgtttcctaagggcctccaaatttgaatggcccgtggagcagaaggtctaagagataggattcattaacctagggaaggttagagctaaaaagcgggcattcgtggggaagaggcctgtaggcatcttgccaacaacccctggcacagctcattcgtctggcttgtccttggcacaggctttttggagcagagaggacgtgaaggagaatgagggctttgctgagtatctgagtgcccttaaaaggtctgtaggagatacgtgtgcgacaggtgcagttttggcggtatctgagggtaacattgataactaatacatgtacagtacttcccagcatgcagatactttgcatgcaccatttgacccactcagcggtcttgtggtgaggtgatagaattatcctcagcagacagaagaggaaactgagtttcagagagatcaagtgacttactcagagttacacagctagttaaaggtgataccgggactggatccctggtctttggataagtcatttgcacttcagtgtacttatcattctgagaccagcctagcgtgatacctctctcagtgagggaaggagaaagtcaactctgatgttttttgtttggttggtttttttttttttttagtttgctgctctttctgattaagagatctcccttctctacttttgttcccagtaccttcccattggactggacattgatattgcccaggatggtctcgaactcctgcactcaagcgatcctcccacctcagcctcccaaagtgttaagattacaggcgttgagccactgcacctggccaacgaggctttctgagatggcatacagtggaaaccgttttcgttcatgcctgaagcagggaacaggagaaacagtagttgaagaagtcagtgagggaagggaccagaatgggcctccttactgtattggaagtggatcatcttccacttccaattcagacactttaatacctctatggtggtattaaagtctcttctcctgactgctggtgctccactgaagcttcctggtgcttgcctgggtcctgaagaatttctgaggatgatacttctccacggaggcgacgtggcatagtattagtattaaaagcagtgtcaaaaatcacaattacttttgcaccaacctaatagttaacagcttttgcttgagggccggctggcctaggcctgaatcctggctctgccatttgctaaattatgtggccctgcacaaattttatgctgtctttagcaatcagtttgacgaattctaaaatggtgatgataattgtacctaggtttattgtgaggatttgaagagatattccttatatagccttcagcgtagtgactggcccgcggcaagcatttaataaattgagctgctgttactgttaccagcaccaccaaatagttacatcgtatcctcacccttgccttggaagagcaagaagtccttctctgtacatctttgttttctttcttatgtcttgcactcgacagtgatttattttcattttattgttttgagcaccaattgtaaaaggagatctcaccgaaggcccagaagccgcctgtgtttctgttgccttagtggagcacttgccttttggttccagacctatcatggtgtaaatttggcctctctgcttaccttctgagtccctcagttttctcattcgttaaaatgctcataataccgcctcccatgcagaagtgttgtgacgcttagagctggagtataaacaatgctgcgtgtcgttggcacagcgtaggtgcttaacaaatggtcgtgatgataatgataaaattcctcttttagggtaaagggggagctagagactggaatgggacattgcattaagtgctactacacgcccttctttttcttccctgcagtcgaggggagctgcagcgggagtcagacttcatggccagtgtagacagcagctggatctcctggggggttggggtcttcctgctgaagcctctcaagtggactctttctaacatgctgggagataataaggttccagctgaggaggtccttgtcgctgtggagctgttgaaggtgggtactcagaagggggtgtctgggtgtcacctggtgtgatcacagcatcctccacgtcccttgtgtgcgttcttgaaaaaagaccgtctctttttttacatggctactgagttgataactgagtttatctgctctcccttgtacttgcactttgtaatattcattagtgggacttgtacatttcaaggggttagcctttgtgtcggaattttcagttaacagtggattattttctagtatctgtagggattaatgttaagcagcagtttaggaaatgctttgccttctgtagtttactgacaagagtttctcaggattcctgaggaccttgtggctttcagcattgccacctctaaactggcccactcagctccactttcctactttgtgaccgtgccctttccccttccagctttctgatggcctgccttctgctgcacatgctcttctccctcgaagaggccactggggccatggtcccgtcctagcctcctggtcagtcagcctcctcccggcctgcctttcccacagccttgatcgcatggctcattgctccactcccttcctcagcaacaccctcagctcctttgcatctcctttggtccacaccaggagactcccagcagatccgtagccctcgatcagccacatggggtttaccacccctgctggagaaaatcgcctgtcatcagactgcggtccccttcatggtccccagctgtgggaggccctcggagccacaccacagccttatttttctttctctctgtcccctcacacgctcattccatcctgtgtcctctttgctctctgcgggcacttcactgagaaagtagactcagcattcctgagtcggtctacagttttctgtcctcttaccccaagcctgacctcctcagagcccacctcctcctgtgctcagagtgtagtgtccttcctctgcctctcgtctgctctgcgtctaccctgcccctcttgtccctttgtcccatcggctggcctcttgcctggtatgttccatctcactctcagcctgtaaacccaaacccactcaatgcttttccgaacaccgattgtctcttttgtggtacctgggccctgcctagtgactgctgtttccttctcaaccgacctttgtgtcaccacttcctacctcccattcactctgtcatctggtgcagacctgcttcaaccaccccctccacccagactgctgtggatgaggtgataggggcctgtgtgtgtgtgtgtgtgtgtgtgtgtgtgtgtgtgtgtgtgtgttgactgagcacctggtgaccaaaggacactgcagagaatagttatttttgaaattcacagaccaagcctgacctcagggcccttgcatagccctagaggcttcacacagctggagccagcccagaatccccacgcctcaccccgggagaagcacccatttctcctcaccttgagagactccagcccactctgcctcagattgaggaagaggtctccaaaatctgccaatctctccctgtctcccaggattgttgactaagcagcccacattttcttcagggcagcccctctgctccatctgcaccccttccccgcacccatgcagttattcccctgcccagtggcctggtctccagggctcagtcagggtccccgcaaagactgcaggatggtttgggtggttctgggccaagctcaggtcctctctgcggtctttctgtcatctcacattgtcctgcagcctggggcagaggagaggtaaacccatggatccccacagatgaggtttgtactggcaaacaaagaaagcctgctgttgaggtaaattttttttcccaagtccagacagagaaacagccagtaactttttacttttggatggtattgaaaaataaaaaaagcaaaaattgtccattattttgttatttaaaaaaaatacaaagaaatgaaagttttctttctgttcatccaacttttctctctttgggtactctcttcccatttgtgtgagagtgcgtgagtgtgtatgtttacagatttatgtttccttaatggctttgtcaactctagttcttcctgaaaatcttattcacttgtttcttgtctgtctttcctactagaagggaacagtgccatgggccctgggaccctgggagcagtgcctgatacctggtagacagtcaataaatacctgttgagtgagtgaatacacacgcacagacacgtgtacacatcactccatttttttctagaaatggaattggtgtgcaccctgctttgttttctttccattgcattgtatcaggtgcctctttccaggccctacacagtatggatttactcgattctttttgatgcctgcatgataatctatcgatatgccacaatttatgcaacaggtgctccgcagatggacatttcaatcattccaatttctctctccatttttccattacaaaaaaatgccacttgtgcagagatctatgtacttctccctttatttttatggaattgatttttcggaaatgcatatgtgccttttcaattttaacaaaaattgccaaactacttttcaaaagtttgtaataagttgcactttcatcaagactgtattagggagtccagtctccccacatccttgtcagcacgggatgacatcagtcttttaaatcttaccaacttattgggaaaaaaaaaatgatacgtcctgtttgtgaattgagcatttttttcacctatgtactagccatttacatatctccttctgtgaattgtctgtttatatcctttcctgttttgtattgtgttatcttttttttttttttgagatggagtttgtcacccaggctggagtgcagtggcacgatctccgctcactgcaagctccgcctttcgggttcacgccattctcctgcttcagccttccgagcagttgggactacaggtgccgccaccacgcccggctaattttttgtatttttagtagagattgggtttcaccatgttagccaggatggtctcgatctcctgacctcgtgatccacccgccttggcctcccaaagtgctgggattacaggtgtgagccaccgcgcctggccttattgtgttatcttttatgttaatctgtattctttatgtgttagagatagatatatataatgtatttagtattaaggatgtaaatattgtttctcagtctgttgcttgcttactttttatttatgatgtcttttaccctatagaaggtttcatatttctgtagtcagatctctcagacttttctgtatggctattgaatttcaggtcttgtttaggaaggcatcttctgactcaagcttaacttattactttgtactttaattttttccgtttacatttccttcaatacttatttttgtgtatgtggtggggaaagctctattttcttccaaatggatagccagttgtctcagtatcttttcttgatgtcgtgtaacattaatttatttgtgtgtggttactgtgcagttactgcagtggttgcatatctggtagggcagtcaaatcaatgaaaactcgatgtctgtgtcttattaggctcttttctaccctttggcactgttgacgagtcacttcttgaaatgtccaatttccttgaatttgtttacatgacgctgcaagttctccttgattcccccaaagcagtccttctgtctctcttttgtggcttccttcacttcctaaacagtggtcttccttcagcttattactttcctccctgaatgtccttgggatctttgctgtggatatgctgaggactttcaacattgtatcgctgtcctgctgtctcctttgagtttcagatttgtatatccactgataggttgtatacataatcttggatatagctcagacatcttatttatgtatttatttatagacagagtctcactctgttgcccaggctgtagtgcagtggcacaatgttggctcagtgcaacctccgcctcctgggttcaagcaattctcctgcctcagcctctcaagtagctgggatcacaggcgctgccaccatggccagctaattttttgtattttttttagtggagatggggtttcaccctgttggccaggctggtctcaaactcctgatctcaggtgatccacccacctcggcctcataaagtgctgggattagaggcgtgaggcacaccgtgcctggcttctctcagacatcttaaaacatcataaccaagatgggatattttgtctttactttaaactaggcttttcctcctgaattttttcctattaatagcatcacatccaccagtcactcaatccatagtgactttccactagactttttcttccaaccctatatccagttaccagtgttatcttctgaattttgtttgtctccaccactcctctttctatgtttattattcaaatagcctgttgttcagacccacataatctcttgtgtaaatggtctccttgcctctaatcttaactctacaatccatcgtctcccaattggcagttagctttctgttaatctagccatgtcagtttccttcagtgaccacccaccatctacagaataaaggatcatcataagatattacactttctataatataggatattacactttctataatataggagtgcttctcctctctctcctttcccctctacatttgtacacgtgccgcattgacccacccttctatgctcatgttcctctctcttcccgagatacccgtttcacctcttcacctaggaacttgctcactcttcagaattctgctcaactttttttttttttcaagaaatcctgaatccccaggcagtccttccctatattttcatagcatcccatggacacctcagtctttcttgtttctggtagtaatttccatgtatgagcgaaatggtgacattatttcttggaacttatatttcttactcttgtcactaatcttatcccatctgtttattatgtacttttctttgttttttgttttgttttgttctgtttgagacagagttttgctcttgtcgcccaggctggagtgcagtggcgcgatctcggctcactgcaacctccgcctcccaggttcaagtgattctcctgcctcaacctcccaagtagctgggattacaggtgcccgccatcacatccagctattttttttttttgtatttttagtaaagacagggttttgccatgttggccaggctggtctcgaactcctgacctcaggtgatcctgacctcaggtgatcagcctcccagagtgctgggattacaggcatgagctgccacgcctggcccttttcccttaaggctttttacagagtcaataatgtttgtgtttgcagtatagccaattggcaggaaaatggagagactacaattggaagggaattagatgtcatttcatccaccctcttcagagagtgatactgaggactagtgaggctgtgactggcccaaggtcacccaggctaggctggaaatcaggcctcagctctcgttcacagctgttctacctcacctcacctcctcagtcttcccttgggaaaccacaggccagtgagaggcaggagtgtaaaatccccgatgggcgtgcagcgcagagctggctcccagcatctcatgtgcctcatttcatgtggagtcaccaaactttcctccaaggcttaaaaacacagatccttcctcctttttgaaaagccgctactttggggaggatggggttgggattcccgggtgcttgccttgtgtgatcatggttttgaaactcagggtcagggtccctgcatcccatggcagtggccagggcctgtcagccaatgttgcctttcccacaggaaaaggctgaggaggtgtatcgtctgtatcagaactcgcccctctcctcccaccccgtggtggccctgtcagagctcagcaccctctgtgctaactcctgcccagatgagaggaccttctacttggtgttgctgcagctgcagaaggagaagagggtcacagtcctcgagcagaacggggagaaggtatggaggctcatctgttcattcactgactcagcagtgattgattaagtacatagagtagtgagcccttatctcagattttggtttccagagtttcagtaacctgccgtcaaccacggtccaaaaataataagtggaaaattccaaacattaacagttccatagatttcagttgtgcactgttctgagtagtgtgatgggacctcgtagtatcccgcctgtatgcgcatccctgctttgtccagcatctccccgcctgagtcacttaggagtcgtctcagtgatcagattgactgtcacagcattgcagtggttgtgttcaagccacccttattgtactttttttttttttttttgagatggagtcttgctctgtcgcccagcaatggcgcaatctgagctcactgcagcctccgcctcccgggttcaagcgattcttctgcctcagcctcccaagtagctggaattacaggcatgcgccacggcccctggctaatttttgtatttttagtagagaaggggtttcaccagttagccaggctgtcttgaactcctgacctcaagtgatctgcctgcctcggcctcccaaagtgctaggattacaggtgtaaaccaccgtgcctggcctattatacttaataatggccccaaagcacaagagcagtggtgctggcagtccagatttaccaaagagaagcggtcaagtgcttcctttaagtgaaaaggtaaaagttctggaccaaataaggaagaccaaatatcatttgctgagtttgctaggatctacagtaagaatgaatcttctataggaaaaattgtgaagaaggaaaaatgtttacatagctatatgtatagggttcagtattctctgagggttcagacctccacaggggattccattcactggaacatatcccccatggataaggggggcctgctgttctcacatgcccaccaccagcgctcctggttgggaatttctcagcctttgtggtagcagcagtagtaatttctccctgtccctcagattgtgaagtttgcccgagggccacgtgccaaggtctctccagtcaatgacgtagatgttggggtgtaccagctgatgcagagtgaacagcttctctcacgcaaagtggagtccttatcccaggaagcagagaggtaacttttaaccctgaactgagcctcctccccactgttgctggcccccagagccagcaaaatgttagtatatgtgggcttttaaaaaataggtgggttttttttttttttagctaattacaaaagtactcactgctcaatgcagaaatcatggaaagcacagaatatcacaaaagagaaaaaaaaatgaggcctctttttttttttttttttttttgagacggagtctcgctctgtcacccgggctggagtgcagtggtgcgatctcggctcactgcaagctccgcctcctgggttcacgccattttcctgcgccagcctcccgagtagctggtactacagccacccgccaccacgcctggcgaattttttatatttctagtagagatggggtttcactgtgttagccaggatggtctgaatctcctgacctcgtgatccgcccgcctctgcctcccaaagtgctgggattacaggcgtgagccaccgtgcccggcctttttttttttttttttcctcaattgagacagggtcttgcactgtcccctaggctggagagcagtgacatgatcttagctcactgcagtctaactcctgagctcaagcaatcttcccgccttagcctcctgagtagctgggaccacaagtgtgcaccaccatgcctggctaatttttaattttttgttgagatggggtctcaccatgttgcccaggctggtctcacactcctcggcgcaaacaagacccccacctcagcctcccaaagtgctgggattactggggtgagccactgtgcccagctgaaaacgtgagtcttaatgcttcccccacagaccatgactgtgaatgttatgtttatgctcttccttgccagtgtccattcaactaatatttattgagccaggcattgtcttagggactggggatacagccgtggacaagacaggtccctgcctttgcagaggtctttctgaggggctcttcccctgcccttccccctgaatgactgtacaaggaatccctaataaagacacaccctctggcaggagaacacagacattgaagtctgtcttccaggtgggaaagtctagccttgttgggaggcagaggatatgtccgaggtcattgttggtgttaacagttccttccctgtctccccaaaacaggaagatgtgtataatgtgccctgtttgcatgttctgtccctgtgggttgaaaaagcttatccttagtcagcctgcttgagcctgccacaacagactgtcaacagacttgccacaagagtttttgccgtcagcatcagttttcttgccactgggtattgggtttagtcttcagatttaaaaccaccttaactgagtgctgctctcagggaccctttgggaccctgccactcccatcctgtggcacagtaatcttatctgtccctttgtttccaggtgtaaagaagaagcccgccgggcatgccgagcaggaaagaagcagctggtgagttcttgtctcctccagacccatagcagtgccccaggcaggcctggagcaatgcccagcagttcagagagcagcttgctggggttttgaggggggtagagtctgcagggggcccaggcaccacagcttattttacagatgggagggaagaaagctgggacatccttagcgtctgggaagggctgtcttcctcttgccctttggctcgcccagttcagcactgacctaagtctccatgcctccaggcactgaggtctctcaaggccaagcaacggacagagaagcgcatcgaggccttgcatgccaagctggacactgttcaaggcatcctggaccggatctatgcctcccagacagatcagatggtagtcactcccctctactccagcacttggctggtctctccgtgtgtgttggtcacttgcagcttcggcttggctgtttggaggatccctgggttctttcgggatgtggctggggttgcctttgcctttccagtcaggaggtttggcctcggtgggtatagggtaaattgagcttgggtgccaaaaaaaacaccccaaagctgccttttggagttgaaacaatattgtatgtattttgccaaatgtctgtcatttgcactgatagctttgctttgtctttaggtttttaacgcctaccaggctggggtaggagcactcaaactctccatgaaggatgtcacagtggagaaggcagagagcctcgtggatcagatccaagaggtacagaaaggggccagggagggacacacagagaaggaggtgacagaggacagatttgacttcattgcacatcctctttaacgaaacctgacttgtgaccttgtgaacttgaggaagctttccttgattctcacttggggcaggattgctttgtaaccctagatattttccaccaaagactgagatgctcagagccaccatgcccagctcaaggctttgcacttgtctcttacagctctgtgacacccaggatgaagtttctcagactctggctggtggggtaacaaatggcttaggtgagtggacaaggtggttatttttatttttattcattttttttttttttttttttttttgagacggagtctcgctctgtcgcccaggctggagtgcagtggcgggatctcggctcactgcaagctccgcctcccgggttcacgccattctcctgcctcagcctcccaagtagctgggactacaggcgcctgccactatgcccggctaattttttgtatttttagtagagacggggtcattttttttttttttagatggagtctcactctgtcacctaggctggagtgcagtgacgcattctcggctcgctgcaacctccgcctcctgggttcaagtgattctcctgcctcagcctcctgagtagctgggattataggcgcccgccaccacgcctggctaatttttgtatttttagtagagatggggtttcacctgttggtcaggctggtctggaactcctgacctcgtgatccgccggcctcggcctcccaaagtgctggggttacaggtgtgagccaccacacctggcccaaggtgagtgtttcttcctttcatcctggaagacttcgcttccctgtgctgtgatgagtttgacagtagatttgcttttccgggtgcaggcaggcctctcctagcctgccctccctggggctcatctgcgggagcagtcagggaattgccatttcctcctcgacagctgtgctcatgacccatctgtgctgcggcattcctcattcacaaggcttctgactggaggatcctccttcaagggcagtgggaatcagggtcaggtccatggtgttaacagaatacacctccttggtgacttagcttggagtgtggaagaatcatctctgcttgaagctaccctcctgacttccgggatttttttttaatctgcaaggaggcctggcatcttttgtaaagctaagcgggttttggtaacctcagtcctgtactcattaatgcatttctcccttgagtttatgcatctttatgttgtcttttctttccagattttgacagtgaagaactggagaaggaattggacatcctccttcaggataccaccaaagaacctttggatctgcctgacaacccccgcaataggcattttaccaacagcgtgcctaaccctaggatctcagatgctgaacttgaagctgaacttgagaaactgtccttatcagagggaggtatggagctgttttccaaggcctttggagggcatatggccttctggctgacagagttgatgggcccaagcccaattactccatttgccagagtaccagagccctgtagggagttgagggttgtttatattaagactcagtctcttaatcactggaatgccttcatcttcaagattttcctgttatagtgttcagtcatttctttgccttgcaggtttggtcccaagcagtaaatctccaaaaaggcaattggaaccgactctaaagccattgtaggaccctcaagtgaaggaccctcatgtaaaagagagaccaggcttgctgggtgtgtacatagttatttaaacaagaaactctcagaatgtgtttggaagaggagaaaggagaaccactgattttatctggatgctactacttactacaggacagatagaatttctggaagcgatgctccaaaggcttgctcccagctgtatcatggacctgccttctcatctttatagtgccacgatttatacagtcctgtgtctgacctgtcatttcatagcctgcagttcttgccattggcacacttagatttgtcttcacccaccagcttcgttccagcccatgaaggggaaagatttgcagctttgccaaatctgaatcagttcccactcccccctgcggttttttagaggggtttatcctgtcgctaatgtcagttaaatagcttattcctgtcccacttgatttcactggtaagaatgagaatgaaatacttaaaggttttatttgaggggacttagctgccattttatggaaaaacatgtagtataaaaggtttttctccagggtcatttgacagaattctccaggtgttcagagaaagaggtcgccccaagtggcccagagtcgtgacagtccggaggggctggtgtggccggtgggcgctgcctcttcatctgatggagccaggaggtccgccggtgggggaaaggcttcttccgaggcttctgcactcgcctgggaagaggcccagtcactacaggaccctgagatgtccagaccccttatgcaaagaggcaaaattgttgccttggccccatcatggagtaacttaaaaatacatatctatcttccagaacaagccacacaacaacacacctcccctccttgagcctggcacacagcaccgggctctctgaatttggaaagtgtttcattttgtggcctactgctcggggcaggggggctgagccaggatgtgcaatagaagcaggggttgtcccctctgccagatctgtggggagctgcccaggccacccgtgcacctgtcctgcttcctgcagtccccactccctgcctcctttgagggactgggggacttggggtagggggacagacgggaaagggtctgaatgctctggcaatggcgagaagaagcgatcgccacactgccactctgctgcccagccaccaggatttctgcctggagccctttgccctgtccctgctgcctgaggtggcgtagagcagaggagaagaatttgctccaggccaggagcacaaatcgttgttcaatgaatttctctccaactcgaccttggtaaacggaaatgttgggggtgaagagaaacaatcactatttttttcttttttatctggtaaataattaaaagaaaaaccgaacacttg

SEQ ID NO: 2 - DNA, 3410 bp, Homo sapiens
gctgcctcctctgactgttatgcagcaggccggcaggtcaataaacttgcttgcctgactttgagtatactcgtcttttctcttggccaaccttacggtagctgtaccagtttgcatttccaccagcaatgaatgagagttcctgttgctctgcatcctcaccagcatttggtgtggtcagtattttggattttcaccattctaatagagattcacggatccatccgggtggtccgggcaccactggcgcccctctgcggctattccccagttccattttctgaagccacaggtcagaagccgctgtacaacgctttgccgaactccagaatcttgaaggagacgtaaggtgcagccacctgccgcgcaggcgcaagcctttctttcggcacaaagaccgtgggaggaggggtcggcgcaagcgctcggtgtctctctgaaaagaacttcatcatactgcctcctggctgacggagcgcagcgcaacgcatgcgccttgaagacttatggaacttatttcacgctcagggcggcggtgacgtgtgaacgagaaggaggtggtcaaggaacggaagccgggagggaacgagggcggaagcggaccagggccaggcttgtgttcgcagccttgccggggctggggttccgatgtggtccccggagcgggaggccgaggccccagccgggggagacccggcgggccttctgccccccgagtgggaggaggacgaggagcgcatgtccttcctgttctccgctttcaagaggagtcgcgaggtgaacagcaccgactgggacagcaagatgggcttctgggcgccgttggtgctgagccacagccgccgccagggggtggtgcgcctgcgtctgcgggacttgcaggaggcctttcagcgcaaggggagcgtcccgctggggctggccacggtgctgcaggacctgctgcgtcgaggggagctgcagcgggagtcagacttcatggccagtgtagacagcagctggatctcctggggggttggggtcttcctgctgaagcctctcaagtggactctttctaacatgctgggagataataaggttccagctgaggaggtccttgtcgctgtggagctgttgaaggaaaaggctgaggaggtgtatcgtctgtatcagaactcgcccctctcctcccaccccgtggtggccctgtcagagctcagcaccctctgtgctaactcctgcccagatgagaggaccttctacttggtgttgctgcagctgcagaaggagaagagggtcacagtcctcgagcagaacggggagaagattgtgaagtttgcccgagggccacgtgccaaggtctctccagtcaatgacgtagatgttggggtgtaccagctgatgcagagtgaacagcttctctcacgcaaagtggagtccttatcccaggaagcagagaggtgtaaagaagaagcccgccgggcatgccgagcaggaaagaagcagctggcactgaggtctctcaaggccaagcaacggacagagaagcgcatcgaggccttgcatgccaagctggacactgttcaaggcatcctggaccggatctatgcctcccagacagatcagatggtttttaacgcctaccaggctggggtaggagcactcaaactctccatgaaggatgtcacagtggagaaggcagagagcctcgtggatcagatccaagagctctgtgacacccaggatgaagtttctcagactctggctggtggggtaacaaatggcttagattttgacagtgaagaactggagaaggaattggacatcctccttcaggataccaccaaagaacctttggatctgcctgacaacccccgcaataggcattttaccaacagcgtgcctaaccctaggatctcagatgctgaacttgaagctgaacttgagaaactgtccttatcagagggaggtttggtcccaagcagtaaatctccaaaaaggcaattggaaccgactctaaagccattgtaggaccctcaagtgaaggaccctcatgtaaaagagagaccaggcttgctgggtgtgtacatagttatttaaacaagaaactctcagaatgtgtttggaagaggagaaaggagaaccactgattttatctggatgctactacttactacaggacagatagaatttctggaagcgatgctccaaaggcttgctcccagctgtatcatggacctgccttctcatctttatagtgccacgatttatacagtcctgtgtctgacctgtcatttcatagcctgcagttcttgccattggcacacttagatttgtcttcacccaccagcttcgttccagcccatgaaggggaaagatttgcagctttgccaaatctgaatcagttcccactcccccctgcggttttttagaggggtttatcctgtcgctaatgtcagttaaatagcttattcctgtcccacttgatttcactggtaagaatgagaatgaaatacttaaaggttttatttgaggggacttagctgccattttatggaaaaacatgtagtataaaaggtttttctccagggtcatttgacagaattctccaggtgttcagagaaagaggtcgccccaagtggcccagagtcgtgacagtccggaggggctggtgtggccggtgggcgctgcctcttcatctgatggagccaggaggtccgccggtgggggaaaggcttcttccgaggcttctgcactcgcctgggaagaggcccagtcactacaggaccctgagatgtccagaccccttatgcaaagaggcaaaattgttgccttggccccatcatggagtaacttaaaaatacatatctatcttccagaacaagccacacaacaacacacctcccctccttgagcctggcacacagcaccgggctctctgaatttggaaagtgtttcattttgtggcctactgctcggggcaggggggctgagccaggatgtgcaatagaagcaggggttgtcccctctgccagatctgtggggagctgcccaggccacccgtgcacctgtcctgcttcctgcagtccccactccctgcctcctttgagggactgggggacttggggtagggggacagacgggaaagggtctgaatgctctggcaatggcgagaagaagcgatcgccacactgccactctgctgcccagccaccaggatttctgcctggagccctttgccctgtccctgctgcctgaggtggcgtagagcagaggagaagaatttgctccaggccaggagcacaaatcgttgttcaatgaatttctctccaactcgaccttggtaaacggaaatgttgggggtgaagagaaacaatcactatttttttcttttttatctggtaaataattaaaagaaaaaccgaacactt

SEQ ID NO: 3-4 - skipped (2 entries)

SEQ ID NO: 5 - DNA, 23 bp, Artificial sequence
  Primer
caagtggactctttctaacatgc

SEQ ID NO: 6 - DNA, 21 bp, Artificial sequence
  Primer
gcgagttctgatacagacgat

SEQ ID NO: 7 - DNA, 26 bp, Artificial sequence
  Probe
cctcctcagccttttccttcaacagc

SEQ ID NO: 8-9 - skipped (2 entries)

SEQ ID NO: 10 - DNA, 20 bp, Artificial sequence
  Synthetic oligonucleotide
ggcattttatattcatggac

SEQ ID NO: 11 - DNA, 20 bp, Artificial sequence
  Synthetic oligonucleotide
aggacagtttctcaagttca

SEQ ID NO: 12 - DNA, 20 bp, Artificial sequence
  Synthetic oligonucleotide
atgtgatgctattaatagga

SEQ ID NO: 13 - DNA, 20 bp, Artificial sequence
  Synthetic oligonucleotide
agtacatagatctctgcaca

SEQ ID NO: 14 - DNA, 20 bp, Artificial sequence
  Synthetic oligonucleotide
ttggatctgatccacgaggc

SEQ ID NO: 15 - DNA, 20 bp, Artificial sequence
  Synthetic oligonucleotide
aaagctgttaactattaggt

SEQ ID NO: 16 - DNA, 20 bp, Artificial sequence
  Synthetic oligonucleotide
gacgagtatactcaaagtca

SEQ ID NO: 17 - DNA, 20 bp, Artificial sequence
  Synthetic oligonucleotide
cccccaacatttccgtttac

SEQ ID NO: 18 - DNA, 20 bp, Artificial sequence
  Synthetic oligonucleotide
gtcaattcccatatacctat

SEQ ID NO: 19 - DNA, 20 bp, Artificial sequence
  Synthetic oligonucleotide
aaggacactacactctgagc

SEQ ID NO: 20 - DNA, 20 bp, Artificial sequence
  Synthetic oligonucleotide
atgatgatcctttattctgt

SEQ ID NO: 21 - DNA, 20 bp, Artificial sequence
  Synthetic oligonucleotide
atcagagttgactttctcct

SEQ ID NO: 22 - DNA, 20 bp, Artificial sequence
  Synthetic oligonucleotide
tcttccacactccaagctaa

SEQ ID NO: 23 - DNA, 20 bp, Artificial sequence
  Synthetic oligonucleotide
aagcctggtctctcttttac

SEQ ID NO: 24 - DNA, 20 bp, Artificial sequence
  Synthetic oligonucleotide
gcatcgcttccagaaattct

SEQ ID NO: 25 - DNA, 20 bp, Artificial sequence
  Synthetic oligonucleotide
tggtccagaacttttacctt

SEQ ID NO: 26 - DNA, 20 bp, Artificial sequence
  Synthetic oligonucleotide
ctcttagaccttctgctcca

SEQ ID NO: 27 - DNA, 20 bp, Artificial sequence
  Synthetic oligonucleotide
aactcagttatcaactcagt

SEQ ID NO: 28 - DNA, 20 bp, Artificial sequence
  Synthetic oligonucleotide
ctgctgtctacactggccat

SEQ ID NO: 29 - DNA, 20 bp, Artificial sequence
  Synthetic oligonucleotide
tccagataaaatcagtggtt

SEQ ID NO: 30 - DNA, 20 bp, Artificial sequence
  Synthetic oligonucleotide
tgctcacaccattcctagtc

SEQ ID NO: 31 - DNA, 20 bp, Artificial sequence
  Synthetic oligonucleotide
atgagcctccataccttctc

SEQ ID NO: 32 - DNA, 20 bp, Artificial sequence
  Synthetic oligonucleotide
tatcgatagattatcatgca

SEQ ID NO: 33 - DNA, 20 bp, Artificial sequence
  Synthetic oligonucleotide
tcgcttcttctcgccattgc

SEQ ID NO: 34 - DNA, 20 bp, Artificial sequence
  Synthetic oligonucleotide
actgtatgccatctcagaaa

SEQ ID NO: 35 - DNA, 20 bp, Artificial sequence
  Synthetic oligonucleotide
tcacgaccatttgttaagca

SEQ ID NO: 36 - DNA, 20 bp, Artificial sequence
  Synthetic oligonucleotide
tccaacaccagctcatgata

SEQ ID NO: 37 - DNA, 20 bp, Artificial sequence
  Synthetic oligonucleotide
cgacctctttctctgaacac

SEQ ID NO: 38 - DNA, 20 bp, Artificial sequence
  Synthetic oligonucleotide
ggaagatgatccacttccaa

SEQ ID NO: 39 - DNA, 20 bp, Artificial sequence
  Synthetic oligonucleotide
actccaagctaagtcaccaa

SEQ ID NO: 40 - DNA, 20 bp, Artificial sequence
  Synthetic oligonucleotide
acagaccttttaagggcact

SEQ ID NO: 41 - DNA, 20 bp, Artificial sequence
  Synthetic oligonucleotide
gcccatcaactctgtcagcc

SEQ ID NO: 42 - DNA, 20 bp, Artificial sequence
  Synthetic oligonucleotide
ccatttcgctcatacatgga

SEQ ID NO: 43 - DNA, 20 bp, Artificial sequence
  Synthetic oligonucleotide
gagggtcctacaatggcttt

SEQ ID NO: 44 - DNA, 20 bp, Artificial sequence
  Synthetic oligonucleotide
ctgcaaatctttccccttca

SEQ ID NO: 45 - DNA, 20 bp, Artificial sequence
  Synthetic oligonucleotide
aagtcctcagcatatccaca

SEQ ID NO: 46 - DNA, 20 bp, Artificial sequence
  Synthetic oligonucleotide
tgtttgccagtacaaacctc

SEQ ID NO: 47 - DNA, 20 bp, Artificial sequence
  Synthetic oligonucleotide
accaaaacccgcttagcttt

SEQ ID NO: 48 - DNA, 20 bp, Artificial sequence
  Synthetic oligonucleotide
gtacagatcttacaccattc

SEQ ID NO: 49 - DNA, 20 bp, Artificial sequence
  Synthetic oligonucleotide
cctgaaattcaatagccata

SEQ ID NO: 50 - DNA, 20 bp, Artificial sequence
  Synthetic oligonucleotide
acccaccttcaacagctcca

SEQ ID NO: 51 - DNA, 20 bp, Artificial sequence
  Synthetic oligonucleotide
catcgagttttcattgattt

SEQ ID NO: 52 - DNA, 20 bp, Artificial sequence
  Synthetic oligonucleotide
aagcccacatatactaacat

SEQ ID NO: 53 - DNA, 20 bp, Artificial sequence
  Synthetic oligonucleotide
ggtcttttttcaagaacgca

SEQ ID NO: 54 - DNA, 20 bp, Artificial sequence
  Synthetic oligonucleotide
aggataagctttttcaaccc

SEQ ID NO: 55 - DNA, 20 bp, Artificial sequence
  Synthetic oligonucleotide
ctactgtttctcctgttccc

SEQ ID NO: 56 - DNA, 20 bp, Artificial sequence
  Synthetic oligonucleotide
gatgtccaattccttctcca

SEQ ID NO: 57 - DNA, 20 bp, Artificial sequence
  Synthetic oligonucleotide
ggtgcacaccaattccattt

SEQ ID NO: 58 - DNA, 20 bp, Artificial sequence
  Synthetic oligonucleotide
tgtccactcacctaagccat

SEQ ID NO: 59 - DNA, 20 bp, Artificial sequence
  Synthetic oligonucleotide
cttcttgctcttccaaggca

SEQ ID NO: 60 - DNA, 20 bp, Artificial sequence
  Synthetic oligonucleotide
ccagatggtttcatttgata

SEQ ID NO: 61 - DNA, 20 bp, Artificial sequence
  Synthetic oligonucleotide
gaggataattctatcacctc

SEQ ID NO: 62 - DNA, 20 bp, Artificial sequence
  Synthetic oligonucleotide
tggagcaaattcttctcctc

SEQ ID NO: 63 - DNA, 20 bp, Artificial sequence
  Synthetic oligonucleotide
ggcacattatacacatcttc

SEQ ID NO: 64 - DNA, 20 bp, Artificial sequence
  Synthetic oligonucleotide
gctcaatttaccctataccc

SEQ ID NO: 65 - DNA, 20 bp, Artificial sequence
  Synthetic oligonucleotide
gatctgtgtttttaagcctt

SEQ ID NO: 66 - DNA, 20 bp, Artificial sequence
  Synthetic oligonucleotide
ctggaaccttattatctccc

SEQ ID NO: 67 - DNA, 20 bp, Artificial sequence
  Synthetic oligonucleotide
gagatgattcttccacactc

SEQ ID NO: 68 - DNA, 20 bp, Artificial sequence
  Synthetic oligonucleotide
gggctcactactctatgtac

SEQ ID NO: 69 - DNA, 20 bp, Artificial sequence
  Synthetic oligonucleotide
tcagcaaatgatatttggtc

SEQ ID NO: 70 - DNA, 20 bp, Artificial sequence
  Synthetic oligonucleotide
tgtacttaatcaatcactgc

SEQ ID NO: 71 - DNA, 20 bp, Artificial sequence
  Synthetic oligonucleotide
ccatctcttttataagatag

SEQ ID NO: 72 - DNA, 20 bp, Artificial sequence
  Synthetic oligonucleotide
gtaactgcacagtaaccaca

SEQ ID NO: 73 - DNA, 20 bp, Artificial sequence
  Synthetic oligonucleotide
ggtgagatctccttttacaa

SEQ ID NO: 74 - DNA, 20 bp, Artificial sequence
  Synthetic oligonucleotide
ctagactttcccacctggaa

SEQ ID NO: 75 - DNA, 20 bp, Artificial sequence
  Synthetic oligonucleotide
tcagaccctttcccgtctgt

SEQ ID NO: 76 - DNA, 20 bp, Artificial sequence
  Synthetic oligonucleotide
gaactgattcagatttggca

SEQ ID NO: 77 - DNA, 20 bp, Artificial sequence
  Synthetic oligonucleotide
ctgaggcctgatttccagcc

SEQ ID NO: 78 - DNA, 20 bp, Artificial sequence
  Synthetic oligonucleotide
atggacctgaccctgattcc

SEQ ID NO: 79 - DNA, 20 bp, Artificial sequence
  Synthetic oligonucleotide
cattgtttatactccagctc

SEQ ID NO: 80 - DNA, 20 bp, Artificial sequence
  Synthetic oligonucleotide
actgacttcttcaactactg

SEQ ID NO: 81 - DNA, 20 bp, Artificial sequence
  Synthetic oligonucleotide
gaagctgttcactctgcatc

SEQ ID NO: 82 - DNA, 20 bp, Artificial sequence
  Synthetic oligonucleotide
agtcttttcaacaatgagca

SEQ ID NO: 83 - DNA, 20 bp, Artificial sequence
  Synthetic oligonucleotide
catctaattcccttccaatt

SEQ ID NO: 84 - DNA, 20 bp, Artificial sequence
  Synthetic oligonucleotide
actagtcctcagtatcactc

SEQ ID NO: 85 - DNA, 20 bp, Artificial sequence
  Synthetic oligonucleotide
agtgattgtttctcttcacc

SEQ ID NO: 86 - DNA, 20 bp, Artificial sequence
  Synthetic oligonucleotide
acggatacaacccaacttca

SEQ ID NO: 87 - DNA, 20 bp, Artificial sequence
  Synthetic oligonucleotide
gggcaaacttcacaatctga

SEQ ID NO: 88 - DNA, 20 bp, Artificial sequence
  Synthetic oligonucleotide
tagtcctcagtatcactctc

SEQ ID NO: 89 - DNA, 20 bp, Artificial sequence
  Synthetic oligonucleotide
cccatatacctataatgatc

SEQ ID NO: 90 - DNA, 20 bp, Artificial sequence
  Synthetic oligonucleotide
agtatactcaaagtcaggca

SEQ ID NO: 91 - DNA, 20 bp, Artificial sequence
  Synthetic oligonucleotide
ccactgcccttgaaggagga

SEQ ID NO: 92 - DNA, 20 bp, Artificial sequence
  Synthetic oligonucleotide
ggataagctttttcaaccca

SEQ ID NO: 93 - DNA, 20 bp, Artificial sequence
  Synthetic oligonucleotide
aagctgttcactctgcatca

SEQ ID NO: 94 - DNA, 20 bp, Artificial sequence
  Synthetic oligonucleotide
tcgcttccagaaattctatc

SEQ ID NO: 95 - DNA, 20 bp, Artificial sequence
  Synthetic oligonucleotide
gtgtattctgttaacaccat

SEQ ID NO: 96 - DNA, 20 bp, Artificial sequence
  Synthetic oligonucleotide
gggattttacactcctgcct

SEQ ID NO: 97 - DNA, 20 bp, Artificial sequence
  Synthetic oligonucleotide
gtcttttttcaagaacgcac

SEQ ID NO: 98 - DNA, 20 bp, Artificial sequence
  Synthetic oligonucleotide
aggataattctatcacctca

SEQ ID NO: 99 - DNA, 20 bp, Artificial sequence
  Synthetic oligonucleotide
tcaacagctccacagcgaca

SEQ ID NO: 100 - DNA, 20 bp, Artificial sequence
  Synthetic oligonucleotide
accattcacaaaagccagct

SEQ ID NO: 101 - DNA, 20 bp, Artificial sequence
  Synthetic oligonucleotide
tgatgatcctttattctgta

SEQ ID NO: 102 - DNA, 20 bp, Artificial sequence
  Synthetic oligonucleotide
tccaagattatgtatacaac

SEQ ID NO: 103 - DNA, 20 bp, Artificial sequence
  Synthetic oligonucleotide
ggctcactactctatgtact

SEQ ID NO: 104 - DNA, 20 bp, Artificial sequence
  Synthetic oligonucleotide
actgtcacctctgttaggct

SEQ ID NO: 105 - DNA, 20 bp, Artificial sequence
  Synthetic oligonucleotide
ttccacactccaagctaagt

SEQ ID NO: 106 - DNA, 20 bp, Artificial sequence
  Synthetic oligonucleotide
acagtttctcaagttcagct

SEQ ID NO: 107 - DNA, 20 bp, Artificial sequence
  Synthetic oligonucleotide
cagatggtttcatttgataa

SEQ ID NO: 108 - DNA, 20 bp, Artificial sequence
  Synthetic oligonucleotide
ttgccagtacaaacctcatc

SEQ ID NO: 109 - DNA, 20 bp, Artificial sequence
  Synthetic oligonucleotide
ggcacatgtaaatacagtca

SEQ ID NO: 110 - DNA, 20 bp, Artificial sequence
  Synthetic oligonucleotide
taagccatttgttaccccac

SEQ ID NO: 111 - DNA, 20 bp, Artificial sequence
  Synthetic oligonucleotide
ccaactgatcctatgaggca

SEQ ID NO: 112 - DNA, 20 bp, Artificial sequence
  Synthetic oligonucleotide
tagctttacaaaagatgcca

SEQ ID NO: 113 - DNA, 20 bp, Artificial sequence
  Synthetic oligonucleotide
gtcttttcaacaatgagcac

SEQ ID NO: 114 - DNA, 20 bp, Artificial sequence
  Synthetic oligonucleotide
gcatatccacagcaaagatc

SEQ ID NO: 115 - DNA, 20 bp, Artificial sequence
  Synthetic oligonucleotide
cctgcttctattgcacatcc

SEQ ID NO: 116 - DNA, 20 bp, Artificial sequence
  Synthetic oligonucleotide
gtatgccatctcagaaagcc

SEQ ID NO: 117 - DNA, 20 bp, Artificial sequence
  Synthetic oligonucleotide
ggtccagaacttttaccttt

SEQ ID NO: 118 - DNA, 20 bp, Artificial sequence
  Synthetic oligonucleotide
ctgacttcttcaactactgt

SEQ ID NO: 119 - DNA, 20 bp, Artificial sequence
  Synthetic oligonucleotide
acctctttctctgaacacct

SEQ ID NO: 120 - DNA, 20 bp, Artificial sequence
  Synthetic oligonucleotide
cctgatttccagcctagcct

SEQ ID NO: 121 - DNA, 20 bp, Artificial sequence
  Synthetic oligonucleotide
tgattgtttctcttcacccc

SEQ ID NO: 122 - DNA, 20 bp, Artificial sequence
  Synthetic oligonucleotide
agatgatccacttccaatac

SEQ ID NO: 123 - DNA, 20 bp, Artificial sequence
  Synthetic oligonucleotide
gccagagcattcagaccctt

SEQ ID NO: 124 - DNA, 20 bp, Artificial sequence
  Synthetic oligonucleotide
gtgcacaccaattccatttc

SEQ ID NO: 125 - DNA, 20 bp, Artificial sequence
  Synthetic oligonucleotide
acttcacaatctgagggaca

SEQ ID NO: 126 - DNA, 20 bp, Artificial sequence
  Synthetic oligonucleotide
tgacactgcttttaatacta

SEQ ID NO: 127 - DNA, 20 bp, Artificial sequence
  Synthetic oligonucleotide
ggagcaaattcttctcctct

SEQ ID NO: 128 - DNA, 20 bp, Artificial sequence
  Synthetic oligonucleotide
agagtcggttccaattgcct

SEQ ID NO: 129 - DNA, 20 bp, Artificial sequence
  Synthetic oligonucleotide
ctgcagcaacaccaagtaga

SEQ ID NO: 130 - DNA, 20 bp, Artificial sequence
  Synthetic oligonucleotide
tactgtttctcctgttccct

SEQ ID NO: 131 - DNA, 20 bp, Artificial sequence
  Synthetic oligonucleotide
agagttgactttctccttcc

SEQ ID NO: 132 - DNA, 20 bp, Artificial sequence
  Synthetic oligonucleotide
cctcttccaaacacattctg

SEQ ID NO: 133 - DNA, 20 bp, Artificial sequence
  Synthetic oligonucleotide
gccaattggctatactgcaa

SEQ ID NO: 134 - DNA, 20 bp, Artificial sequence
  Synthetic oligonucleotide
cgatagattatcatgcaggc

SEQ ID NO: 135 - DNA, 20 bp, Artificial sequence
  Synthetic oligonucleotide
ccaacaccagctcatgataa

SEQ ID NO: 136 - DNA, 20 bp, Artificial sequence
  Synthetic oligonucleotide
gatctccttttacaattggt

SEQ ID NO: 137 - DNA, 20 bp, Artificial sequence
  Synthetic oligonucleotide
tgcacagtaaccacacacaa

SEQ ID NO: 138 - DNA, 20 bp, Artificial sequence
  Synthetic oligonucleotide
ctggtctctcttttacatga

SEQ ID NO: 139 - DNA, 20 bp, Artificial sequence
  Synthetic oligonucleotide
actcagttatcaactcagta

SEQ ID NO: 140 - DNA, 20 bp, Artificial sequence
  Synthetic oligonucleotide
cccaacatttccgtttacca

SEQ ID NO: 141 - DNA, 20 bp, Artificial sequence
  Synthetic oligonucleotide
catgaagtctgactcccgct

SEQ ID NO: 142 - DNA, 20 bp, Artificial sequence
  Synthetic oligonucleotide
tgtccaattccttctccagt

SEQ ID NO: 143 - DNA, 20 bp, Artificial sequence
  Synthetic oligonucleotide
ttcttgctcttccaaggcaa

SEQ ID NO: 144 - DNA, 20 bp, Artificial sequence
  Synthetic oligonucleotide
gccatatgccctccaaaggc

SEQ ID NO: 145 - DNA, 20 bp, Artificial sequence
  Synthetic oligonucleotide
gtacttaatcaatcactgct

SEQ ID NO: 146 - DNA, 20 bp, Artificial sequence
  Synthetic oligonucleotide
gcacattatacacatcttcc

SEQ ID NO: 147 - DNA, 20 bp, Artificial sequence
  Synthetic oligonucleotide
ctgtacgggaaatcctagct

SEQ ID NO: 148 - DNA, 20 bp, Artificial sequence
  Synthetic oligonucleotide
cagcaaatgatatttggtct

SEQ ID NO: 149 - DNA, 20 bp, Artificial sequence
  Synthetic oligonucleotide
gtagatccagtgaaatccca

SEQ ID NO: 150 - DNA, 20 bp, Artificial sequence
  Synthetic oligonucleotide
ctgtgccaacgacacgcagc

SEQ ID NO: 151 - DNA, 20 bp, Artificial sequence
  Synthetic oligonucleotide
gcccacatatactaacattt

SEQ ID NO: 152 - DNA, 20 bp, Artificial sequence
  Synthetic oligonucleotide
tgtgacatccttcatggaga

SEQ ID NO: 153 - DNA, 20 bp, Artificial sequence
  Synthetic oligonucleotide
aactgattcagatttggcaa

SEQ ID NO: 154 - DNA, 20 bp, Artificial sequence
  Synthetic oligonucleotide
tttcattgatttgactgccc

SEQ ID NO: 155 - DNA, 20 bp, Artificial sequence
  Synthetic oligonucleotide
aggacactacactctgagca

SEQ ID NO: 156 - DNA, 20 bp, Artificial sequence
  Synthetic oligonucleotide
atgctatgaaaatataggga

SEQ ID NO: 157 - DNA, 20 bp, Artificial sequence
  Synthetic oligonucleotide
gtgcaacttattacaaactt

SEQ ID NO: 158 - DNA, 20 bp, Artificial sequence
  Synthetic oligonucleotide
acagacttcaatgtctgtgt

SEQ ID NO: 159 - DNA, 20 bp, Artificial sequence
  Synthetic oligonucleotide
tgcaagtgaccaacacacac

SEQ ID NO: 160 - DNA, 20 bp, Artificial sequence
  Synthetic oligonucleotide
attgtttatactccagctct

SEQ ID NO: 161 - DNA, 20 bp, Artificial sequence
  Synthetic oligonucleotide
aatctttccccttcatgggc

SEQ ID NO: 162 - DNA, 20 bp, Artificial sequence
  Synthetic oligonucleotide
gtacatagatctctgcacaa

SEQ ID NO: 163 - DNA, 20 bp, Artificial sequence
  Synthetic oligonucleotide
agatgattcttccacactcc

SEQ ID NO: 164 - DNA, 20 bp, Artificial sequence
  Synthetic oligonucleotide
tggcaagatgcctacaggcc

SEQ ID NO: 165 - DNA, 20 bp, Artificial sequence
  Synthetic oligonucleotide
tggaaccttattatctccca

SEQ ID NO: 166 - DNA, 20 bp, Artificial sequence
  Synthetic oligonucleotide
gttgtaccagttctgtgcca

SEQ ID NO: 167 - DNA, 20 bp, Artificial sequence
  Synthetic oligonucleotide
atgatcctttattctgtaga

SEQ ID NO: 168 - DNA, 20 bp, Artificial sequence
  Synthetic oligonucleotide
ttgtttatactccagctcta

SEQ ID NO: 169 - DNA, 20 bp, Artificial sequence
  Synthetic oligonucleotide
tttgagtgctcctaccccag

SEQ ID NO: 170 - DNA, 20 bp, Artificial sequence
  Synthetic oligonucleotide
tcctcagtatcactctctga

SEQ ID NO: 171 - DNA, 20 bp, Artificial sequence
  Synthetic oligonucleotide
tcacagcctcactagtcctc

SEQ ID NO: 172 - DNA, 20 bp, Artificial sequence
  Synthetic oligonucleotide
agacgatacacctcctcagc

SEQ ID NO: 173 - DNA, 20 bp, Artificial sequence
  Synthetic oligonucleotide
tccacactccaagctaagtc

SEQ ID NO: 174 - DNA, 20 bp, Artificial sequence
  Synthetic oligonucleotide
aagcggaatttattacagct

SEQ ID NO: 175 - DNA, 20 bp, Artificial sequence
  Synthetic oligonucleotide
cactgcccttgaaggaggat

SEQ ID NO: 176 - DNA, 20 bp, Artificial sequence
  Synthetic oligonucleotide
tatactaacattttgctggc

SEQ ID NO: 177 - DNA, 20 bp, Artificial sequence
  Synthetic oligonucleotide
ccaagattatgtatacaacc

SEQ ID NO: 178 - DNA, 20 bp, Artificial sequence
  Synthetic oligonucleotide
gcacatgtaaatacagtcaa

SEQ ID NO: 179 - DNA, 20 bp, Artificial sequence
  Synthetic oligonucleotide
ggtgtgtctttattagggat

SEQ ID NO: 180 - DNA, 20 bp, Artificial sequence
  Synthetic oligonucleotide
ttcattgatttgactgccct

SEQ ID NO: 181 - DNA, 20 bp, Artificial sequence
  Synthetic oligonucleotide
gctgcttaacattaatccct

SEQ ID NO: 182 - DNA, 20 bp, Artificial sequence
  Synthetic oligonucleotide
gagtcggttccaattgcctt

SEQ ID NO: 183 - DNA, 20 bp, Artificial sequence
  Synthetic oligonucleotide
taactatgtacacacccagc

SEQ ID NO: 184 - DNA, 20 bp, Artificial sequence
  Synthetic oligonucleotide
atgccatctcagaaagcctc

SEQ ID NO: 185 - DNA, 20 bp, Artificial sequence
  Synthetic oligonucleotide
aagccatttgttaccccacc

SEQ ID NO: 186 - DNA, 20 bp, Artificial sequence
  Synthetic oligonucleotide
tgcaacttattacaaacttt

SEQ ID NO: 187 - DNA, 20 bp, Artificial sequence
  Synthetic oligonucleotide
gcatctgagatcctagggtt

SEQ ID NO: 188 - DNA, 20 bp, Artificial sequence
  Synthetic oligonucleotide
aggccttggaaaacagctcc

SEQ ID NO: 189 - DNA, 20 bp, Artificial sequence
  Synthetic oligonucleotide
gattgtttctcttcaccccc

SEQ ID NO: 190 - DNA, 20 bp, Artificial sequence
  Synthetic oligonucleotide
ccattcacaaaagccagctc

SEQ ID NO: 191 - DNA, 20 bp, Artificial sequence
  Synthetic oligonucleotide
gagttgactttctccttccc

SEQ ID NO: 192 - DNA, 20 bp, Artificial sequence
  Synthetic oligonucleotide
gagaaattactactgctgct

SEQ ID NO: 193 - DNA, 20 bp, Artificial sequence
  Synthetic oligonucleotide
ccaggcctccttgcagatta

SEQ ID NO: 194 - DNA, 20 bp, Artificial sequence
  Synthetic oligonucleotide
ggattttacactcctgcctc

SEQ ID NO: 195 - DNA, 20 bp, Artificial sequence
  Synthetic oligonucleotide
gatgattcttccacactcca

SEQ ID NO: 196 - DNA, 20 bp, Artificial sequence
  Synthetic oligonucleotide
tccctcactgacttcttcaa

SEQ ID NO: 197 - DNA, 20 bp, Artificial sequence
  Synthetic oligonucleotide
tctccttttacaattggtgc

SEQ ID NO: 198 - DNA, 20 bp, Artificial sequence
  Synthetic oligonucleotide
atgcaatgtcccattccagt

SEQ ID NO: 199 - DNA, 20 bp, Artificial sequence
  Synthetic oligonucleotide
tattctgttaacaccatgga

SEQ ID NO: 200 - DNA, 20 bp, Artificial sequence
  Synthetic oligonucleotide
cttgaccgcttctctttggt

SEQ ID NO: 201 - DNA, 20 bp, Artificial sequence
  Synthetic oligonucleotide
gagggacacatcctccactt

SEQ ID NO: 202 - DNA, 20 bp, Artificial sequence
  Synthetic oligonucleotide
tgacttcttcaactactgtt

SEQ ID NO: 203 - DNA, 20 bp, Artificial sequence
  Synthetic oligonucleotide
tctctcttttacatgagggt

SEQ ID NO: 204 - DNA, 20 bp, Artificial sequence
  Synthetic oligonucleotide
atccagtgaaatcccagtga

SEQ ID NO: 205 - DNA, 20 bp, Artificial sequence
  Synthetic oligonucleotide
atcgagtaaatccatactgt

SEQ ID NO: 206 - DNA, 20 bp, Artificial sequence
  Synthetic oligonucleotide
gcaaagctgcaaatctttcc

SEQ ID NO: 207 - DNA, 20 bp, Artificial sequence
  Synthetic oligonucleotide
gagaagtatcatcctcagaa

SEQ ID NO: 208 - DNA, 20 bp, Artificial sequence
  Synthetic oligonucleotide
catttccgtttaccaaggtc

SEQ ID NO: 209 - DNA, 20 bp, Artificial sequence
  Synthetic oligonucleotide
tccaggtacagatcttacac

SEQ ID NO: 210 - DNA, 20 bp, Artificial sequence
  Synthetic oligonucleotide
acagatgagcctccatacct

SEQ ID NO: 211 - DNA, 20 bp, Artificial sequence
  Synthetic oligonucleotide
cgtgtgtattcactcactca

SEQ ID NO: 212 - DNA, 20 bp, Artificial sequence
  Synthetic oligonucleotide
acctaggtacaattatcatc

SEQ ID NO: 213 - DNA, 20 bp, Artificial sequence
  Synthetic oligonucleotide
gggaaatcctagctaacaca

SEQ ID NO: 214 - DNA, 20 bp, Artificial sequence
  Synthetic oligonucleotide
tttggtcttccttatttggt

SEQ ID NO: 215 - DNA, 20 bp, Artificial sequence
  Synthetic oligonucleotide
aggaactgttaacaccaaca

SEQ ID NO: 216 - DNA, 20 bp, Artificial sequence
  Synthetic oligonucleotide
aaaccttttatactacatgt

SEQ ID NO: 217 - DNA, 20 bp, Artificial sequence
  Synthetic oligonucleotide
tgccagtacaaacctcatct

SEQ ID NO: 218 - DNA, 20 bp, Artificial sequence
  Synthetic oligonucleotide
ttggctatactgcaaacaca

SEQ ID NO: 219 - DNA, 20 bp, Artificial sequence
  Synthetic oligonucleotide
attcttctcctctgctctac

SEQ ID NO: 220 - DNA, 20 bp, Artificial sequence
  Synthetic oligonucleotide
gtattagttatcaatgttac

SEQ ID NO: 221 - DNA, 20 bp, Artificial sequence
  Synthetic oligonucleotide
cacatttcttgaaagcacac

SEQ ID NO: 222 - DNA, 20 bp, Artificial sequence
  Synthetic oligonucleotide
gctcactactctatgtactt

SEQ ID NO: 223 - DNA, 20 bp, Artificial sequence
  Synthetic oligonucleotide
gctccacagcgacaaggacc

SEQ ID NO: 224 - DNA, 20 bp, Artificial sequence
  Synthetic oligonucleotide
atagagctttccccaccaca

SEQ ID NO: 225 - DNA, 20 bp, Artificial sequence
  Synthetic oligonucleotide
gtgaccaacacacacggaga

SEQ ID NO: 226 - DNA, 20 bp, Artificial sequence
  Synthetic oligonucleotide
cgtcatgtaaacaaattcaa

SEQ ID NO: 227 - DNA, 20 bp, Artificial sequence
  Synthetic oligonucleotide
gaaccttattatctcccagc

SEQ ID NO: 228 - DNA, 20 bp, Artificial sequence
  Synthetic oligonucleotide
ctccagttcttcactgtcaa

SEQ ID NO: 229 - DNA, 20 bp, Artificial sequence
  Synthetic oligonucleotide
agttatcaactcagtagcca

SEQ ID NO: 230 - DNA, 20 bp, Artificial sequence
  Synthetic oligonucleotide
cgcagcattgtttatactcc

SEQ ID NO: 231 - DNA, 20 bp, Artificial sequence
  Synthetic oligonucleotide
aacaattttgcctctttgca

SEQ ID NO: 232 - DNA, 20 bp, Artificial sequence
  Synthetic oligonucleotide
ggcaagcaagtttattgacc

SEQ ID NO: 233 - DNA, 20 bp, Artificial sequence
  Synthetic oligonucleotide
tcttttttcaagaacgcaca

SEQ ID NO: 234 - DNA, 20 bp, Artificial sequence
  Synthetic oligonucleotide
cactctgcatcagctggtac

SEQ ID NO: 235 - DNA, 20 bp, Artificial sequence
  Synthetic oligonucleotide
agtgtaatatcttatgatga

SEQ ID NO: 236 - DNA, 20 bp, Artificial sequence
  Synthetic oligonucleotide
atctttccccttcatgggct

SEQ ID NO: 237 - DNA, 20 bp, Artificial sequence
  Synthetic oligonucleotide
gataagctttttcaacccac

SEQ ID NO: 238 - DNA, 20 bp, Artificial sequence
  Synthetic oligonucleotide
ccagagcattcagacccttt

SEQ ID NO: 239 - DNA, 20 bp, Artificial sequence
  Synthetic oligonucleotide
ggtataaatctaacacggta

SEQ ID NO: 240 - DNA, 20 bp, Artificial sequence
  Synthetic oligonucleotide
ttgaaatgtccatctgcgga

SEQ ID NO: 241 - DNA, 20 bp, Artificial sequence
  Synthetic oligonucleotide
gctttttagctctaaccttc

SEQ ID NO: 242 - DNA, 20 bp, Artificial sequence
  Synthetic oligonucleotide
gcatgaacgaaaacggtttc

SEQ ID NO: 243 - DNA, 20 bp, Artificial sequence
  Synthetic oligonucleotide
tccagaaattctatctgtcc

SEQ ID NO: 244 - DNA, 20 bp, Artificial sequence
  Synthetic oligonucleotide
ttggtcttccttatttggtc

SEQ ID NO: 245 - DNA, 20 bp, Artificial sequence
  Synthetic oligonucleotide
aagccagctcaaatggatca

SEQ ID NO: 246 - DNA, 20 bp, Artificial sequence
  Synthetic oligonucleotide
gctagattaacagaaagcta

SEQ ID NO: 247 - DNA, 20 bp, Artificial sequence
  Synthetic oligonucleotide
aaccttattatctcccagca

SEQ ID NO: 248 - DNA, 20 bp, Artificial sequence
  Synthetic oligonucleotide
gcaatatcaatgtccagtcc

SEQ ID NO: 249 - DNA, 20 bp, Artificial sequence
  Synthetic oligonucleotide
cttctcctctgctctacgcc

SEQ ID NO: 250 - DNA, 20 bp, Artificial sequence
  Synthetic oligonucleotide
caagattatgtatacaacct

SEQ ID NO: 251 - DNA, 20 bp, Artificial sequence
  Synthetic oligonucleotide
attttacactcctgcctctc

SEQ ID NO: 252 - DNA, 20 bp, Artificial sequence
  Synthetic oligonucleotide
agagcattcagaccctttcc

SEQ ID NO: 253 - DNA, 20 bp, Artificial sequence
  Synthetic oligonucleotide
cagatgagcctccatacctt

SEQ ID NO: 254 - DNA, 20 bp, Artificial sequence
  Synthetic oligonucleotide
cacaacacttctgcatggga

SEQ ID NO: 255 - DNA, 20 bp, Artificial sequence
  Synthetic oligonucleotide
cggtatgtacaaattctaca

SEQ ID NO: 256 - DNA, 20 bp, Artificial sequence
  Synthetic oligonucleotide
gggcgacctctttctctgaa

SEQ ID NO: 257 - DNA, 20 bp, Artificial sequence
  Synthetic oligonucleotide
gaaaacggtttccactgtat

SEQ ID NO: 258 - DNA, 20 bp, Artificial sequence
  Synthetic oligonucleotide
ctctcttttacatgagggtc

SEQ ID NO: 259 - DNA, 20 bp, Artificial sequence
  Synthetic oligonucleotide
attgaacaacgatttgtgct

SEQ ID NO: 260 - DNA, 20 bp, Artificial sequence
  Synthetic oligonucleotide
tcattgatttgactgcccta

SEQ ID NO: 261 - DNA, 20 bp, Artificial sequence
  Synthetic oligonucleotide
ctgcaggctatgaaatgaca

SEQ ID NO: 262 - DNA, 20 bp, Artificial sequence
  Synthetic oligonucleotide
tgcaatgtcccattccagtc

SEQ ID NO: 263 - DNA, 20 bp, Artificial sequence
  Synthetic oligonucleotide
actactctatgtacttaatc

SEQ ID NO: 264 - DNA, 20 bp, Artificial sequence
  Synthetic oligonucleotide
atgctgtgatcacaccaggt

SEQ ID NO: 265 - DNA, 20 bp, Artificial sequence
  Synthetic oligonucleotide
gcaacttattacaaactttt

SEQ ID NO: 266 - DNA, 20 bp, Artificial sequence
  Synthetic oligonucleotide
cggacatatcctctgcctcc

SEQ ID NO: 267 - DNA, 20 bp, Artificial sequence
  Synthetic oligonucleotide
gacttcttcaactactgttt

SEQ ID NO: 268 - DNA, 20 bp, Artificial sequence
  Synthetic oligonucleotide
ttgaccgcttctctttggta

SEQ ID NO: 269 - DNA, 20 bp, Artificial sequence
  Synthetic oligonucleotide
tagctaacacagttagccaa

SEQ ID NO: 270 - DNA, 20 bp, Artificial sequence
  Synthetic oligonucleotide
gggacacatcctccacttga

SEQ ID NO: 271 - DNA, 20 bp, Artificial sequence
  Synthetic oligonucleotide
acccaagctcaatttaccct

SEQ ID NO: 272 - DNA, 20 bp, Artificial sequence
  Synthetic oligonucleotide
gtaatatcttatgatgatcc

SEQ ID NO: 273 - DNA, 20 bp, Artificial sequence
  Synthetic oligonucleotide
gtcaggcagatccaaaggtt

SEQ ID NO: 274 - DNA, 20 bp, Artificial sequence
  Synthetic oligonucleotide
gtatcatcctcagaaattct

SEQ ID NO: 275 - DNA, 20 bp, Artificial sequence
  Synthetic oligonucleotide
cacaatttttcctatagaag

SEQ ID NO: 276 - DNA, 20 bp, Artificial sequence
  Synthetic oligonucleotide
tcagctggtacaccccaaca

SEQ ID NO: 277 - DNA, 20 bp, Artificial sequence
  Synthetic oligonucleotide
atgtcaccatttcgctcata

SEQ ID NO: 278 - DNA, 20 bp, Artificial sequence
  Synthetic oligonucleotide
agcggaatttattacagcta

SEQ ID NO: 279 - DNA, 20 bp, Artificial sequence
  Synthetic oligonucleotide
tccagttcttcactgtcaaa

SEQ ID NO: 280 - DNA, 20 bp, Artificial sequence
  Synthetic oligonucleotide
ttggacatttcaagaagtga

SEQ ID NO: 281 - DNA, 20 bp, Artificial sequence
  Synthetic oligonucleotide
agacggtcttttttcaagaa

SEQ ID NO: 282 - DNA, 20 bp, Artificial sequence
  Synthetic oligonucleotide
atgattcttccacactccaa

SEQ ID NO: 283 - DNA, 20 bp, Artificial sequence
  Synthetic oligonucleotide
aaagctgcaaatctttcccc

SEQ ID NO: 284 - DNA, 20 bp, Artificial sequence
  Synthetic oligonucleotide
cctgttgcataaattgtggc

SEQ ID NO: 285 - DNA, 20 bp, Artificial sequence
  Synthetic oligonucleotide
tccatgggtttacctctcct

SEQ ID NO: 286 - DNA, 20 bp, Artificial sequence
  Synthetic oligonucleotide
acagtcaactgacctttcga

SEQ ID NO: 287 - DNA, 20 bp, Artificial sequence
  Synthetic oligonucleotide
agggattccttgtacagtca

SEQ ID NO: 288 - DNA, 20 bp, Artificial sequence
  Synthetic oligonucleotide
gcaagcaagtttattgacct

SEQ ID NO: 289 - DNA, 20 bp, Artificial sequence
  Synthetic oligonucleotide
catttgttaccccaccagcc

SEQ ID NO: 290 - DNA, 20 bp, Artificial sequence
  Synthetic oligonucleotide
atcccagtgaaaatcatggc

SEQ ID NO: 291 - DNA, 20 bp, Artificial sequence
  Synthetic oligonucleotide
ctgtgccatctcttttataa

SEQ ID NO: 292 - DNA, 20 bp, Artificial sequence
  Synthetic oligonucleotide
ctcagccttttcctgtggga

SEQ ID NO: 293 - DNA, 20 bp, Artificial sequence
  Synthetic oligonucleotide
gcttaacattaatccctaca

SEQ ID NO: 294 - DNA, 20 bp, Artificial sequence
  Synthetic oligonucleotide
ccaggtacagatcttacacc

SEQ ID NO: 295 - DNA, 20 bp, Artificial sequence
  Synthetic oligonucleotide
aggcacctgatacaatgcaa

SEQ ID NO: 296 - DNA, 20 bp, Artificial sequence
  Synthetic oligonucleotide
cctaggtacaattatcatca

SEQ ID NO: 297 - DNA, 20 bp, Artificial sequence
  Synthetic oligonucleotide
gcctcactagtcctcagtat

SEQ ID NO: 298 - DNA, 20 bp, Artificial sequence
  Synthetic oligonucleotide
atactaacattttgctggct

SEQ ID NO: 299 - DNA, 20 bp, Artificial sequence
  Synthetic oligonucleotide
ccctcactgacttcttcaac

SEQ ID NO: 300 - DNA, 20 bp, Artificial sequence
  Synthetic oligonucleotide
tgtttctcttcacccccaac

SEQ ID NO: 301 - DNA, 20 bp, Artificial sequence
  Synthetic oligonucleotide
gcacactctgccaaaacaca

SEQ ID NO: 302 - DNA, 20 bp, Artificial sequence
  Synthetic oligonucleotide
ttagttatcaatgttaccct

SEQ ID NO: 303 - DNA, 20 bp, Artificial sequence
  Synthetic oligonucleotide
aggttaatgaatcctatctc

SEQ ID NO: 304 - DNA, 20 bp, Artificial sequence
  Synthetic oligonucleotide
tagagctttccccaccacat

SEQ ID NO: 305 - DNA, 20 bp, Artificial sequence
  Synthetic oligonucleotide
gtcccggtatcacctttaac

SEQ ID NO: 306 - DNA, 20 bp, Artificial sequence
  Synthetic oligonucleotide
ccacactccaagctaagtca

SEQ ID NO: 307 - DNA, 20 bp, Artificial sequence
  Synthetic oligonucleotide
gcagagatgattcttccaca

SEQ ID NO: 308 - DNA, 20 bp, Artificial sequence
  Synthetic oligonucleotide
tgtcaaactcatcacagcac

SEQ ID NO: 309 - DNA, 20 bp, Artificial sequence
  Synthetic oligonucleotide
tcggttccaattgccttttt

SEQ ID NO: 310 - DNA, 20 bp, Artificial sequence
  Synthetic oligonucleotide
aaccttttatactacatgtt

SEQ ID NO: 311 - DNA, 20 bp, Artificial sequence
  Synthetic oligonucleotide
attgactgtctaccaggtat

SEQ ID NO: 312 - DNA, 20 bp, Artificial sequence
  Synthetic oligonucleotide
aactatgtacacacccagca

SEQ ID NO: 313 - DNA, 20 bp, Artificial sequence
  Synthetic oligonucleotide
cagcattgtttatactccag

SEQ ID NO: 314 - DNA, 20 bp, Artificial sequence
  Synthetic oligonucleotide
cctcagtatcactctctgaa

SEQ ID NO: 315 - DNA, 20 bp, Artificial sequence
  Synthetic oligonucleotide
aggatgccttgaacagtgtc

SEQ ID NO: 316 - DNA, 20 bp, Artificial sequence
  Synthetic oligonucleotide
ataagctttttcaacccaca

SEQ ID NO: 317 - DNA, 20 bp, Artificial sequence
  Synthetic oligonucleotide
cagctggaaccttattatct

SEQ ID NO: 318 - DNA, 20 bp, Artificial sequence
  Synthetic oligonucleotide
tggaaaacagctccatacct

SEQ ID NO: 319 - DNA, 20 bp, Artificial sequence
  Synthetic oligonucleotide
ccagaaattctatctgtcct

SEQ ID NO: 320 - DNA, 20 bp, Artificial sequence
  Synthetic oligonucleotide
gaaggacttcttgctcttcc

SEQ ID NO: 321 - DNA, 20 bp, Artificial sequence
  Synthetic oligonucleotide
attctgttaacaccatggac

SEQ ID NO: 322 - DNA, 20 bp, Artificial sequence
  Synthetic oligonucleotide
tggtcttccttatttggtcc

SEQ ID NO: 323 - DNA, 20 bp, Artificial sequence
  Synthetic oligonucleotide
gaggatgtccaattccttct

SEQ ID NO: 324 - DNA, 20 bp, Artificial sequence
  Synthetic oligonucleotide
ttttacactcctgcctctca

SEQ ID NO: 325 - DNA, 20 bp, Artificial sequence
  Synthetic oligonucleotide
gcaatgtcccattccagtct

SEQ ID NO: 326 - DNA, 20 bp, Artificial sequence
  Synthetic oligonucleotide
caatatcaatgtccagtcca

SEQ ID NO: 327 - DNA, 20 bp, Artificial sequence
  Synthetic oligonucleotide
agtacccaccttcaacagct

SEQ ID NO: 328 - DNA, 20 bp, Artificial sequence
  Synthetic oligonucleotide
cagctggtacaccccaacat

SEQ ID NO: 329 - DNA, 20 bp, Artificial sequence
  Synthetic oligonucleotide
gccaaatttacaccatgata

SEQ ID NO: 330 - DNA, 20 bp, Artificial sequence
  Synthetic oligonucleotide
tactaacattttgctggctc

SEQ ID NO: 331 - DNA, 20 bp, Artificial sequence
  Synthetic oligonucleotide
agcattgtttatactccagc

SEQ ID NO: 332 - DNA, 20 bp, Artificial sequence
  Synthetic oligonucleotide
cagagatgattcttccacac

SEQ ID NO: 333 - DNA, 20 bp, Artificial sequence
  Synthetic oligonucleotide
gtcaccatttcgctcataca

SEQ ID NO: 334 - DNA, 20 bp, Artificial sequence
  Synthetic oligonucleotide
ggtccttcacttgagggtcc

SEQ ID NO: 335 - DNA, 20 bp, Artificial sequence
  Synthetic oligonucleotide
ttgaacaacgatttgtgctc

SEQ ID NO: 336 - DNA, 20 bp, Artificial sequence
  Synthetic oligonucleotide
ggcgacctctttctctgaac

SEQ ID NO: 337 - DNA, 20 bp, Artificial sequence
  Synthetic oligonucleotide
accttattatctcccagcat

SEQ ID NO: 338 - DNA, 20 bp, Artificial sequence
  Synthetic oligonucleotide
gtgccatctcttttataaga

SEQ ID NO: 339 - DNA, 20 bp, Artificial sequence
  Synthetic oligonucleotide
gaagacataattatccatgc

SEQ ID NO: 340 - DNA, 20 bp, Artificial sequence
  Synthetic oligonucleotide
aaacggtttccactgtatgc

SEQ ID NO: 341 - DNA, 20 bp, Artificial sequence
  Synthetic oligonucleotide
tgaccgcttctctttggtaa

SEQ ID NO: 342 - DNA, 20 bp, Artificial sequence
  Synthetic oligonucleotide
gagcattcagaccctttccc

SEQ ID NO: 343 - DNA, 20 bp, Artificial sequence
  Synthetic oligonucleotide
ttctgttaacaccatggacc

SEQ ID NO: 344 - DNA, 20 bp, Artificial sequence
  Synthetic oligonucleotide
atgtacacacccagcaagcc

SEQ ID NO: 345 - DNA, 20 bp, Artificial sequence
  Synthetic oligonucleotide
tgttaccctcagataccgcc

SEQ ID NO: 346 - DNA, 20 bp, Artificial sequence
  Synthetic oligonucleotide
cacactccaagctaagtcac

SEQ ID NO: 347 - DNA, 20 bp, Artificial sequence
  Synthetic oligonucleotide
gcagggtgcacaccaattcc

SEQ ID NO: 348 - DNA, 20 bp, Artificial sequence
  Synthetic oligonucleotide
tatcacctttaactagctgt

SEQ ID NO: 349 - DNA, 20 bp, Artificial sequence
  Synthetic oligonucleotide
cttcactgtcaaaatctgga

SEQ ID NO: 350 - DNA, 20 bp, Artificial sequence
  Synthetic oligonucleotide
gcaaggagaccatttacaca

SEQ ID NO: 351 - DNA, 20 bp, Artificial sequence
  Synthetic oligonucleotide
gattcttccacactccaagc

SEQ ID NO: 352 - DNA, 20 bp, Artificial sequence
  Synthetic oligonucleotide
ttcctatagaagattcattc

SEQ ID NO: 353 - DNA, 20 bp, Artificial sequence
  Synthetic oligonucleotide
gacacagacatcgagttttc

SEQ ID NO: 354 - DNA, 20 bp, Artificial sequence
  Synthetic oligonucleotide
gtttctcttcacccccaaca

SEQ ID NO: 355 - DNA, 20 bp, Artificial sequence
  Synthetic oligonucleotide
gtagcatccagataaaatca

SEQ ID NO: 356 - DNA, 20 bp, Artificial sequence
  Synthetic oligonucleotide
agatgagcctccataccttc

SEQ ID NO: 357 - DNA, 20 bp, Artificial sequence
  Synthetic oligonucleotide
accaccttgtccactcacct

SEQ ID NO: 358 - DNA, 20 bp, Artificial sequence
  Synthetic oligonucleotide
cctcactgacttcttcaact

SEQ ID NO: 359 - DNA, 20 bp, Artificial sequence
  Synthetic oligonucleotide
actctatgtacttaatcaat

SEQ ID NO: 360 - DNA, 20 bp, Artificial sequence
  Synthetic oligonucleotide
atcttatgatgatcctttat

SEQ ID NO: 361 - DNA, 20 bp, Artificial sequence
  Synthetic oligonucleotide
cctcactagtcctcagtatc

SEQ ID NO: 362 - DNA, 20 bp, Artificial sequence
  Synthetic oligonucleotide
actcaccagctgcttctttc

SEQ ID NO: 363 - DNA, 20 bp, Artificial sequence
  Synthetic oligonucleotide
agctgcaaatctttcccctt

SEQ ID NO: 364 - DNA, 20 bp, Artificial sequence
  Synthetic oligonucleotide
gctttttcaacccacaggga

SEQ ID NO: 365 - DNA, 20 bp, Artificial sequence
  Synthetic oligonucleotide
tggcactgttcccttctagt

SEQ ID NO: 366 - DNA, 20 bp, Artificial sequence
  Synthetic oligonucleotide
tcccagtgaaaatcatggct

SEQ ID NO: 367 - DNA, 20 bp, Artificial sequence
  Synthetic oligonucleotide
gacaggtcagacacaggact

SEQ ID NO: 368 - DNA, 20 bp, Artificial sequence
  Synthetic oligonucleotide
aagctcaatttaccctatac

SEQ ID NO: 369 - DNA, 20 bp, Artificial sequence
  Synthetic oligonucleotide
gacggtcttttttcaagaac

SEQ ID NO: 370 - DNA, 20 bp, Artificial sequence
  Synthetic oligonucleotide
accaaagaagatctaccaac

SEQ ID NO: 371 - DNA, 20 bp, Artificial sequence
  Synthetic oligonucleotide
aaaccatgatcacacaaggc

SEQ ID NO: 372 - DNA, 20 bp, Artificial sequence
  Synthetic oligonucleotide
tcttcaactactgtttctcc

SEQ ID NO: 373 - DNA, 20 bp, Artificial sequence
  Synthetic oligonucleotide
aatgtctgctcacaccattc

SEQ ID NO: 374 - DNA, 20 bp, Artificial sequence
  Synthetic oligonucleotide
cagaccacttgaatatgttt

SEQ ID NO: 375 - DNA, 20 bp, Artificial sequence
  Synthetic oligonucleotide
gcggaatttattacagctag

SEQ ID NO: 376 - DNA, 20 bp, Artificial sequence
  Synthetic oligonucleotide
acaaggctagactttcccac

SEQ ID NO: 377 - DNA, 20 bp, Artificial sequence
  Synthetic oligonucleotide
ccatgggtttacctctcctc

SEQ ID NO: 378 - DNA, 20 bp, Artificial sequence
  Synthetic oligonucleotide
tggatgaaatgacatctaat

SEQ ID NO: 379 - DNA, 20 bp, Artificial sequence
  Synthetic oligonucleotide
ctgattgctaaagacagcat

SEQ ID NO: 380 - DNA, 20 bp, Artificial sequence
  Synthetic oligonucleotide
atggcagctaagtcccctca

SEQ ID NO: 381 - DNA, 20 bp, Artificial sequence
  Synthetic oligonucleotide
ggtatgtacaaattctacat

SEQ ID NO: 382 - DNA, 20 bp, Artificial sequence
  Synthetic oligonucleotide
agagactttaataccaccat

SEQ ID NO: 383 - DNA, 20 bp, Artificial sequence
  Synthetic oligonucleotide
gcatatcgatagattatcat

SEQ ID NO: 384 - DNA, 20 bp, Artificial sequence
  Synthetic oligonucleotide
gcgatcgcttcttctcgcca

SEQ ID NO: 385 - DNA, 20 bp, Artificial sequence
  Synthetic oligonucleotide
taacattcacagtcatggtc

SEQ ID NO: 386 - DNA, 20 bp, Artificial sequence
  Synthetic oligonucleotide
tccgaaaaatcaattccata

SEQ ID NO: 387 - DNA, 20 bp, Artificial sequence
  Synthetic oligonucleotide
aaggacttcttgctcttcca

SEQ ID NO: 388 - DNA, 20 bp, Artificial sequence
  Synthetic oligonucleotide
agctggaaccttattatctc

SEQ ID NO: 389 - DNA, 20 bp, Artificial sequence
  Synthetic oligonucleotide
cctaccagatatgcaaccac

SEQ ID NO: 390 - DNA, 20 bp, Artificial sequence
  Synthetic oligonucleotide
gagctttccccaccacatac

SEQ ID NO: 391 - DNA, 20 bp, Artificial sequence
  Synthetic oligonucleotide
cctttctgtacctcttggat

SEQ ID NO: 392 - DNA, 20 bp, Artificial sequence
  Synthetic oligonucleotide
gaaaacagctccatacctcc

SEQ ID NO: 393 - DNA, 20 bp, Artificial sequence
  Synthetic oligonucleotide
gtatacaacctatcagtgga

SEQ ID NO: 394 - DNA, 20 bp, Artificial sequence
  Synthetic oligonucleotide
ggttaatgaatcctatctct

SEQ ID NO: 395 - DNA, 20 bp, Artificial sequence
  Synthetic oligonucleotide
aggtacagatcttacaccat

SEQ ID NO: 396 - DNA, 20 bp, Artificial sequence
  Synthetic oligonucleotide
tcccactaatgaatattaca

SEQ ID NO: 397 - DNA, 20 bp, Artificial sequence
  Synthetic oligonucleotide
cagtcaactgacctttcgaa

SEQ ID NO: 398 - DNA, 20 bp, Artificial sequence
  Synthetic oligonucleotide
agcaagtttattgacctgcc

SEQ ID NO: 399 - DNA, 20 bp, Artificial sequence
  Synthetic oligonucleotide
tccaattgcctttttggaga

SEQ ID NO: 400 - DNA, 20 bp, Artificial sequence
  Synthetic oligonucleotide
gcaggctttctttgtttgcc

SEQ ID NO: 401 - DNA, 20 bp, Artificial sequence
  Synthetic oligonucleotide
agtcaattcccatataccta

SEQ ID NO: 402 - DNA, 20 bp, Artificial sequence
  Synthetic oligonucleotide
gggtgcacaccaattccatt

SEQ ID NO: 403 - DNA, 20 bp, Artificial sequence
  Synthetic oligonucleotide
agctcaatttaccctatacc

SEQ ID NO: 404 - DNA, 20 bp, Artificial sequence
  Synthetic oligonucleotide
agtatatgataatatgccca

SEQ ID NO: 405 - DNA, 20 bp, Artificial sequence
  Synthetic oligonucleotide
gcattcagaccctttcccgt

SEQ ID NO: 406 - DNA, 20 bp, Artificial sequence
  Synthetic oligonucleotide
cttattatctcccagcatgt

SEQ ID NO: 407 - DNA, 20 bp, Artificial sequence
  Synthetic oligonucleotide
tggtacaccccaacatctac

SEQ ID NO: 408 - DNA, 20 bp, Artificial sequence
  Synthetic oligonucleotide
aggtcccatcacactactca

SEQ ID NO: 409 - DNA, 20 bp, Artificial sequence
  Synthetic oligonucleotide
tcagacacaggactgtataa

SEQ ID NO: 410 - DNA, 20 bp, Artificial sequence
  Synthetic oligonucleotide
tgccttcctaaacaagacct

SEQ ID NO: 411 - DNA, 20 bp, Artificial sequence
  Synthetic oligonucleotide
ctttctgtacctcttggatc

SEQ ID NO: 412 - DNA, 20 bp, Artificial sequence
  Synthetic oligonucleotide
aaccatgatcacacaaggca

SEQ ID NO: 413 - DNA, 20 bp, Artificial sequence
  Synthetic oligonucleotide
gaaagtcctcagcatatcca

SEQ ID NO: 414 - DNA, 20 bp, Artificial sequence
  Synthetic oligonucleotide
acactccaagctaagtcacc

SEQ ID NO: 415 - DNA, 20 bp, Artificial sequence
  Synthetic oligonucleotide
gatgagcctccataccttct

SEQ ID NO: 416 - DNA, 20 bp, Artificial sequence
  Synthetic oligonucleotide
gctgcaaatctttccccttc

SEQ ID NO: 417 - DNA, 20 bp, Artificial sequence
  Synthetic oligonucleotide
gagactttaataccaccata

SEQ ID NO: 418 - DNA, 20 bp, Artificial sequence
  Synthetic oligonucleotide
tcatcacgaccatttgttaa

SEQ ID NO: 419 - DNA, 20 bp, Artificial sequence
  Synthetic oligonucleotide
tgtacacacccagcaagcct

SEQ ID NO: 420 - DNA, 20 bp, Artificial sequence
  Synthetic oligonucleotide
acacagacatcgagttttca

SEQ ID NO: 421 - DNA, 20 bp, Artificial sequence
  Synthetic oligonucleotide
cggtgagatctccttttaca

SEQ ID NO: 422 - DNA, 20 bp, Artificial sequence
  Synthetic oligonucleotide
aactactgtttctcctgttc

SEQ ID NO: 423 - DNA, 20 bp, Artificial sequence
  Synthetic oligonucleotide
gcgacctctttctctgaaca

SEQ ID NO: 424 - DNA, 20 bp, Artificial sequence
  Synthetic oligonucleotide
aggatgtccaattccttctc

SEQ ID NO: 425 - DNA, 20 bp, Artificial sequence
  Synthetic oligonucleotide
atccagataaaatcagtggt

SEQ ID NO: 426 - DNA, 20 bp, Artificial sequence
  Synthetic oligonucleotide
cggtcttttttcaagaacgc

SEQ ID NO: 427 - DNA, 20 bp, Artificial sequence
  Synthetic oligonucleotide
aaggacagtttctcaagttc

SEQ ID NO: 428 - DNA, 20 bp, Artificial sequence
  Synthetic oligonucleotide
tcaccatttcgctcatacat

SEQ ID NO: 429 - DNA, 20 bp, Artificial sequence
  Synthetic oligonucleotide
cgatcgcttcttctcgccat

SEQ ID NO: 430 - DNA, 20 bp, Artificial sequence
  Synthetic oligonucleotide
cactgacttcttcaactact

SEQ ID NO: 431 - DNA, 20 bp, Artificial sequence
  Synthetic oligonucleotide
agacagcataaaatttgtgc

SEQ ID NO: 432 - DNA, 20 bp, Artificial sequence
  Synthetic oligonucleotide
ttaacaccatggacctgacc

SEQ ID NO: 433 - DNA, 20 bp, Artificial sequence
  Synthetic oligonucleotide
gctgaggcctgatttccagc

SEQ ID NO: 434 - DNA, 20 bp, Artificial sequence
  Synthetic oligonucleotide
gctttccatgatttctgcat

SEQ ID NO: 435 - DNA, 20 bp, Artificial sequence
  Synthetic oligonucleotide
agcagataaactcagttatc

SEQ ID NO: 436 - DNA, 20 bp, Artificial sequence
  Synthetic oligonucleotide
tctcattcttaccagtgaaa

SEQ ID NO: 437 - DNA, 20 bp, Artificial sequence
  Synthetic oligonucleotide
gactgaggttaccaaaaccc

SEQ ID NO: 438 - DNA, 20 bp, Artificial sequence
  Synthetic oligonucleotide
ttatgatgatcctttattct

SEQ ID NO: 439 - DNA, 20 bp, Artificial sequence
  Synthetic oligonucleotide
gtacaaattctacataagga

SEQ ID NO: 440 - DNA, 20 bp, Artificial sequence
  Synthetic oligonucleotide
gtacccaccttcaacagctc

SEQ ID NO: 441 - DNA, 20 bp, Artificial sequence
  Synthetic oligonucleotide
gaatttattacagctaggca

SEQ ID NO: 442 - DNA, 20 bp, Artificial sequence
  Synthetic oligonucleotide
attcttccacactccaagct

SEQ ID NO: 443 - DNA, 20 bp, Artificial sequence
  Synthetic oligonucleotide
tgcttctttcctgctcggca

SEQ ID NO: 444 - DNA, 20 bp, Artificial sequence
  Synthetic oligonucleotide
accacttgaatatgtttata

SEQ ID NO: 445 - DNA, 20 bp, Artificial sequence
  Synthetic oligonucleotide
gtttattgacctgccggcct

SEQ ID NO: 446 - DNA, 20 bp, Artificial sequence
  Synthetic oligonucleotide
agtcttaatataaacaaccc

SEQ ID NO: 447 - DNA, 20 bp, Artificial sequence
  Synthetic oligonucleotide
ggacggatacaacccaactt

SEQ ID NO: 448 - DNA, 20 bp, Artificial sequence
  Synthetic oligonucleotide
atatcgatagattatcatgc

SEQ ID NO: 449 - DNA, 20 bp, Artificial sequence
  Synthetic oligonucleotide
tcacccccaacatttccgtt

SEQ ID NO: 450 - DNA, 20 bp, Artificial sequence
  Synthetic oligonucleotide
gctagactttcccacctgga

SEQ ID NO: 451 - DNA, 20 bp, Artificial sequence
  Synthetic oligonucleotide
cctggagcaaattcttctcc

SEQ ID NO: 452 - DNA, 20 bp, Artificial sequence
  Synthetic oligonucleotide
ctcagcaaatgatatttggt

SEQ ID NO: 453 - DNA, 20 bp, Artificial sequence
  Synthetic oligonucleotide
ggtacagatcttacaccatt

SEQ ID NO: 454 - DNA, 20 bp, Artificial sequence
  Synthetic oligonucleotide
gctggaaccttattatctcc

SEQ ID NO: 455 - DNA, 20 bp, Artificial sequence
  Synthetic oligonucleotide
tgccatctcttttataagat

SEQ ID NO: 456 - DNA, 20 bp, Artificial sequence
  Synthetic oligonucleotide
aagtacatagatctctgcac

SEQ ID NO: 457 - DNA, 20 bp, Artificial sequence
  Synthetic oligonucleotide
acttcttgctcttccaaggc

SEQ ID NO: 458 - DNA, 20 bp, Artificial sequence
  Synthetic oligonucleotide
gggcacattatacacatctt

SEQ ID NO: 459 - DNA, 20 bp, Artificial sequence
  Synthetic oligonucleotide
gaatcctatctcttagacct

SEQ ID NO: 460 - DNA, 20 bp, Artificial sequence
  Synthetic oligonucleotide
gtggaaagtcactatggatt

SEQ ID NO: 461 - DNA, 20 bp, Artificial sequence
  Synthetic oligonucleotide
atgacatctaattcccttcc

SEQ ID NO: 462 - DNA, 20 bp, Artificial sequence
  Synthetic oligonucleotide
gtgctcagtcaacacacaca

SEQ ID NO: 463 - DNA, 20 bp, Artificial sequence
  Synthetic oligonucleotide
ccaccttgtccactcaccta

SEQ ID NO: 464 - DNA, 20 bp, Artificial sequence
  Synthetic oligonucleotide
gcattgtttatactccagct

SEQ ID NO: 465 - DNA, 20 bp, Artificial sequence
  Synthetic oligonucleotide
catcagagttgactttctcc

SEQ ID NO: 466 - DNA, 20 bp, Artificial sequence
  Synthetic oligonucleotide
gtatctcctacagacctttt

SEQ ID NO: 467 - DNA, 20 bp, Artificial sequence
  Synthetic oligonucleotide
ctgctcacaccattcctagt

SEQ ID NO: 468 - DNA, 20 bp, Artificial sequence
  Synthetic oligonucleotide
ttggtccagaacttttacct

SEQ ID NO: 469 - DNA, 20 bp, Artificial sequence
  Synthetic oligonucleotide
ttgagggtcctacaatggct

SEQ ID NO: 470 - DNA, 20 bp, Artificial sequence
  Synthetic oligonucleotide
tcactagtcctcagtatcac

SEQ ID NO: 471 - DNA, 20 bp, Artificial sequence
  Synthetic oligonucleotide
agagatgattcttccacact

SEQ ID NO: 472 - DNA, 20 bp, Artificial sequence
  Synthetic oligonucleotide
actgcagtaactgcacagta

SEQ ID NO: 473 - DNA, 20 bp, Artificial sequence
  Synthetic oligonucleotide
atgtacttaatcaatcactg

SEQ ID NO: 474 - DNA, 20 bp, Artificial sequence
  Synthetic oligonucleotide
acggtttccactgtatgcca

SEQ ID NO: 475 - DNA, 20 bp, Artificial sequence
  Synthetic oligonucleotide
gagtcttttcaacaatgagc

SEQ ID NO: 476 - DNA, 20 bp, Artificial sequence
  Synthetic oligonucleotide
cgggcttcttctttacacct

SEQ ID NO: 477 - DNA, 20 bp, Artificial sequence
  Synthetic oligonucleotide
tgaggataattctatcacct